(19) United States
(12) Patent Application Publication
(10) Pub. No.: US 2021/0098083 A1
(43) Pub. Date: Apr. 1, 2021

(54) SYSTEMS AND METHODS FOR COMPLEX BIOMOLECULE SAMPLING AND BIOMARKER DISCOVERY

(71) Applicant: SEER, INC., South San Francisco, CA (US)

(72) Inventors: Philip MA, San Jose, CA (US); Theo PLATT, Danville, CA (US); Omid FAROKHZAD, Waban, MA (US); Gregory Charles TROIANO, South San Francisco, CA (US)

(21) Appl. No.: 17/068,135

(22) Filed: Oct. 12, 2020

Related U.S. Application Data
(63) Continuation of application No. PCT/US2019/028809, filed on Apr. 23, 2019.
(60) Provisional application No. 62/661,388, filed on Apr. 23, 2018.
(60) Provisional application No. 62/824,281, filed on Mar. 26, 2019.

(51) Int. Cl. G16B 50/30; G16B 25/10; G16B 40/00

(52) U.S. Cl. CPC G16B 50/30; G16B 40/00; G16B 25/10

(57) ABSTRACT

Provided herein relates to methods and systems of a complex biomolecule sampling using machine learning algorithms. The methods and systems provided herein can aid in selection of previously unknown biomarkers and provide a report comprising a score or probability relating to a specified biological state. The methods and systems provided herein can aid in the rational design of particles to capture biomarkers.

### CROSS-REFERENCE

This application is a Continuation of International Application No. PCT/US2019/028809, filed Apr. 23, 2019, which claims the benefit of U.S. Provisional Application No. 62/661,388, filed Apr. 23, 2018, and U.S. Provisional Application No. 62/824,281 filed Mar. 26, 2019, each of which which are incorporated herein by reference in their entireties for all purposes.

### BACKGROUND

In the public domain, there are numerous references and well-curated databases that associate various biomolecules (e.g., protein) as biomarkers to many different diseases, disorders, and biological states. Advances in bioinformatics analyses offer new insights into the complex biomolecule changes that take place during the spectrum of health and disease. Yet the sensitivity and specificity of the identified biomarkers have not been adequate for early detection of cancers, for example. This may be attributed to the following; 1) often a single biomarker is not enough to generate sufficient signal to associate with a specific disease state, and 2) even if multiple biomarkers have been identified, classification using these biomarkers is often impaired due to noise from fluctuations in levels of these biomarkers and highly abundant serum/plasms proteins such as albumin. Several attempts have been made to enhance detection of a biomarker present at a low level, such as depletion of highly abundant proteins, isobaric labeling at the peptide level for multiplexed relative quantification, post-depletion plasma fractionation strategies, biomarker harvesting techniques, mathematical approaches for analyzing high quality data set, and multiplexed workflow. Despite all these efforts for improving the sensitivity and specificity of biomarkers, the complex biomolecule sampling has not been robustly successful in early detection of a disease state, e.g., cancer.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

Provided herein is a computer-implemented method for detecting one or more biomarkers in a multi-omic data set, comprising: (a) providing a multi-omic data generated from one or more complex biological samples obtained from one or more individuals, each individual having one or more specified biological states; (b) applying a trained model to the multi-omic data to generate one or more classification model weights, wᵢ . . . wₙ, for one or more features, fᵢ . . . fₙ, yielding (wᵢ, fᵢ), . . . (wₙ, fₙ) and storing (wᵢ, fᵢ), . . . (wₙ, fₙ); (c) querying a reference data set for the one or more features, fᵢ . . . fₙ, to generate a set of scores, sᵢ . . . sₙ, yielding (sᵢ, fᵢ), . . . , (sₙ, fₙ) and storing (sᵢ, fᵢ), . . . (sₙ, fₙ); (d) combining at least (wᵢ, fᵢ), . . . (wₙ, fₙ) and (sᵢ, fᵢ), . . . , (sₙ, fₙ) to generate (wᵢ, sᵢ), . . . (wₙ, sₙ) and selecting a subset of (wᵢ, sᵢ), . . . (wₙ, sₙ) to detect one or more biomarkers. In some embodiments, selecting the subject in (d) comprises filtering (wᵢ, sᵢ), . . . (wₙ, sₙ) such that w at least meets a first threshold and s at least meets a second threshold such that the one or more biomarkers comprise a subset (wₖ, sₖ) . . . (wₘ, sₘ) of (wᵢ, sᵢ), . . . (wₙ, sₙ). In some embodiments, k≥i. In some embodiments, m≤n. In some embodiments, the trained model is trained using a set of labeled multi-omic data of a plurality of complex biological samples, wherein the labeled multi-omic data set comprises the one or more features fᵢ . . . fₙ corresponding to one or more specified biological states, bᵢ . . . bₙ, wherein the one or more features are proteins.

In some embodiments, the computer-implemented method for detecting one or more biomarkers in a multi-omic data set provided herein further comprises obtaining the one or more complex biological samples from the one or more individuals. In some embodiments, the computer-implemented method for detecting one or more biomarkers in a multi-omic data set provided herein further comprises generating an output. In some embodiments, the output corresponds to a specified biological state of the one or more specified biological states. In some embodiments, the reference data set is a database comprising features related to specified biological states by an association score. In some embodiments, the set of scores, sᵢ . . . sₙ, are association scores between the one or more features and the one or more specified biological states. In some embodiments, the one or more complex biological samples are selected from the group consisting of are plasma, serum, whole blood, amniotic fluid, cerebral spinal fluid, urine, saliva, tears, and feces.

In some embodiments, the multi-omic data comprises one or more selected from the group consisting of: proteomic data, genomic data, lipidomic data, glycomic data, transcriptomic data, or metabolomics data. In some embodiments, the multi-omic data comprises proteomic data. In some embodiments, the proteome data comprises (i) protein identifiers and (ii) specified biological states for the one or more individuals. In some embodiments, the multi-omic data is generated by assaying a complex biological sample of an individual of the one or more individuals. In some embodiments, the one or more features represent different proteins. In some embodiments, the one or more complex biological samples are not subjected to protein depletion. In some embodiments, the one or more complex biological samples are subjected to prior protein depletion. In some embodiments, the one or more specified biological states are bᵢ . . . bₙ.

Provided herein is a method of proteome sampling, the method comprising: generating data from a first plasma proteome from a first complex biological sample and a second plasma proteome from a second complex biological sample, wherein the first complex biological sample is from a test subject with a specified biological state and the second complex biological sample is from a reference subject without the specified biological state; and building a trained classification model by extracting a plurality of features comprising a first feature of the first plasma proteome and a second feature of the second plasma proteome, wherein the trained classification model of the first feature and the second feature identifies one or more biomarkers linked to the specified biological state.

In some embodiments, the first plasma proteome differs from the second plasma proteome. In some embodiments, the first complex biological sample and/or the second complex biological sample are not subjected to prior protein depletion. In some embodiments, the first complex biological sample and/or the second complex biological sample are subjected to prior protein depletion. In some embodiments, the method of proteome sampling as provided herein further comprises subjecting the first complex biological sample and/or second complex biological sample to protein depletion prior to generating data. In some embodiments, the first plasma proteome and the second plasma proteome are generated after albumin depletion.

Provided herein is a method of complex biomolecule sampling, the method comprising: generating data from a first biomolecule corona from a first complex biological sample and a second biomolecule corona from a second complex biological sample, wherein the first complex biological sample is from a test subject with a specified biological state and the second complex biological sample is from a reference subject without the specified biological state; and building a trained classification model by extracting a plurality of features comprising a first feature of the first biomolecule corona and a second feature of the second biomolecule corona, wherein the trained classification model of the first feature and the second feature identifies one or more biomarkers linked to the specified biological state.

In some embodiments, associations between particles and captured biomarkers can be organized in a relational database. The relational database can be used to design particles to capture specific biomarkers.

In some embodiments, the biomolecule is selected from the group consisting of proteins, polypeptides, amino acids, sugars, carbohydrates, lipids, fatty acids, steroids, hormones, antibodies, metabolites, and polynucleotides. In some embodiments, the one or more biomarkers are present in a low or previously non-recorded concentration in the first complex biological sample. In some embodiments, the low or previously non-recorded concentration is less than 0.001 μg/ml or non-reported or non-detected in public databases. In some embodiments, the one or more biomarkers are detected with a sensitivity of 70% or more. In some embodiments, the one or more biomarkers are detected with a sensitivity of 90% or more. In some embodiments, the one or more biomarkers are detected with a sensitivity of at least 95%. In some embodiments, the one or more biomarkers are detected with a specificity of 70% or more. In some embodiments, the one or more biomarkers are detected with a specificity of 90% or more. In some embodiments, the one or more biomarkers are detected with a specificity of at least 95%. In some embodiments, the one or more biomarkers is selected from the group consisting of CPN1, FCN3, SAA4, IGHG1, IGHG3, CFHR5, C4B, IGLL5, APOD, SERPINA10, CPN2, FGL1, AHSG, ITIH2, HIST1H4D, C4A, CP, CD5L, CNN2, HRNR, GPLD1, IGKC, MASP2, ITIH1, CFHR1, COLEC10, BIN2, SAA2, ANGPTL6, CFB, TPI1, IGHA2, APOC2, EMILIN1, SBSN, PRG4, PPIF, CFHR2, ORMI, AMYIC, NEXN, CALML5, SERPINA7, IGHM, TUFM, APCS, SLC2A3, TMSB4X, CPQ, and SNCA. In some embodiments, the one or more biomarkers are selected from any one of proteins in Table 1.

In some embodiments, the test subject is a plurality of test subjects, and wherein each of the plurality of test subjects has the specified biological state. In some embodiments, the specified biological state is a disease state, a poor clinical outcome, a good clinical outcome, a high risk of disease, a low risk of disease, a complete response to a treatment, a partial response to a treatment, a stable disease state, or a non-response to a treatment. In some embodiments, the test subject is asymptomatic for the disease state. In some embodiments, the disease state is cancer, cardiovascular disease, endocrine disease, inflammatory disease, or a neurological disease. In some embodiments, the disease state is cancer and the cancer is selected from the group consisting of lung cancer, pancreas cancer, blood cancer, breast cancer, bladder cancer, ovarian cancer, thyroid cancer, brain cancer, prostate cancer, gynecological cancer, adenocarcinoma, sarcoma, neuroendocrine cancer, and gastric cancer.

In some embodiments, the proteome sampling or the complex biomolecule sampling as provided herein comprises a corona on a plurality of particles, wherein at least one particle of the plurality is a nanoparticle. In some embodiments, at least one of the plurality of particles is selected from the group consisting of a polymeric particle, a metal oxide particle, a plasmonic particle, a biomolecule particle, a magnetite particle, a maghemite particle, a micelles, a liposome, an iron oxide particle, a graphene, a silica, a protein-based particle, a DNA-based particle, a DNA-aptamer based particle, a RNA-based particle, a RNA-aptamer based particle, a polystyrene particle, a silver particle, and a gold particle, a quantum dot, a palladium particle, a platinum particle, a titanium particle, a superparamagnetic particle, and any combination thereof. In some embodiments, the plurality of nanoparticles are iron oxide nanoparticles with RNA on the surface. In some embodiments, the plurality of particles are iron oxide/polystyrene nanoparticles with RNA on the surface. In some embodiments, the plurality of particles are polystyrene nanoparticles with RNA on the surface. In some embodiments, the plurality of particles are gold nanoparticles with RNA on the surface. In some embodiments, particles can be made of any combination of any material disclosed herein. In some embodiments, the at least one of the plurality of particles is a liposome, and the liposome comprises at least one of a cationic lipid, an anionic lipid, a neutral lipid, or any combination thereof.

In some embodiments, the liposome comprises the cationic lipid, and the cationic lipid is selected from the group consisting of: N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol); N-(1-(2,3-dioleoyloxy)propyl)-N-2-(sperminecarboxamido)ethyl)-N,N-dimethy-lammonium trifluoracetate (DOSPA); dioctadecylamidoglycyl carboxyspermine (DOGS); 1,2-dioleoyl-3-dimethylammonium propane (DODAP); N,N-dimethyl-2,3-dioleoyloxy)propylamine (DODMA); N-(1,2-dimyristyloxyprop-3-yl)-N,N-dimethyl-N-hydroxyethyl ammonium bromide (DMRIE); 1,2-dioleoyl-sn-3-phosphoethanolamine (DOPE); N-(1-(2,3-dioleyloxy)propyl)-N-(2-(sperminecarboxamido)ethyl)-N,N-dimethy-lammonium trifluoroacetate (DOSPA); dioctadecylamidoglycyl carboxyspermine (DOGS); 1,2-ditetradecanoyl-sn-glycero-3-phosphocholine (DMPC); 1,2-dilinoleyloxy-N,N-dimethylaminopropane (DLinDMA); 1,2-dilinolenyloxy-N,N-dimethylaminopropane (DLenDMA); and any combination thereof.

In some embodiments, the liposome comprises the neutral lipid, and the neutral lipid is selected from the group consisting of diaclphosphatidylcholines, diacylphosphatidylethanolamines, ceramides, sphingomyelins, dihydrosphingomyelins, cephalins, and cerebrosides. In some embodiments, the liposome comprises the neutral lipid, and the neutral lipid is selected from the group consisting of: distearoylphosphatidylcholine (DSPC); dioleoylphosphatidylcholine (DOPC); dipalmitoylphosphatidylcholine (DPPC); dioleoylphosphatidylglycerol (DOPG); dipalmitoylphosphatidylglycerol (DPPG); dioleoyl-phosphatidylethanolamine (DOPE); palmitoyloleoylphosphatidylcholine (POPC); palmitoyloleoyl-phosphatidylethanolamine (POPE); dioleoyl-phosphatidylethanolamine 4-(N-maleimidomethyl)-cyclohexane-1-carboxylate (DOPE-mal); dipalmitoyl phosphatidyl ethanolamine (DPPE); dimyristoylphosphoethanolamine (DMPE); distearoyl-phosphatidylethanolamine (DSPE); 1-stearioyl-2-oleoyl-phosphatidyethanol amine (SOPE); 1,2-dielaidoyl-sn-glycero-3-phophoethanolamine (transDOPE); and 2-distearoyl-sn-glycero-3-phosphocholine (DSPC).

In some embodiments, the liposome comprises the anionic lipid, and the anionic lipid is selected from the group consisting of phosphatidylglycerol, cardiolipin diacylphosphatidylserine, diacylphosphatidic acid, N-dodecanoylphosphatidylethanolamines, N-succinylphosphatidylethanolamines, N-glutarylphosphatidylethanolamines, lysylphosphatidyiglycerols, palmitoyloleyolphosphatidylglycerol (POPG), and other anionic modifying groups joined to neutral lipids. In some embodiments, the liposome is selected from the group consisting of DOPG (1,2-dioleosl-sn-glycero-3-phospho(1′-rac-glycerol), DOTAP (1,2-dioleiyl-3-trimethylammonium propane), DOPE (dioleoylphosphatidylethaneolamine), CHOL (DOPC-cholesterol), and any combination thereof. In some aspects, the at least one particle of the plurality of particles is a nanoparticle. In some aspects, the plurality of particles comprises one or more nanoparticles. In some aspects, the plurality of particles is a plurality of nanoparticles.

Provided herein is a computer-implemented system for complex biomolecule sampling, the computer-implemented system comprising: a first memory unit for receiving a plurality of biomolecule sampling data, wherein the plurality of biomolecule sampling data comprises first biomolecule sampling data from a first complex biological sample and second biomolecule sampling data from a second complex biological sample, wherein the first complex biological sample is from one or more subjects with a specified biological state and the second complex biological sample is from one or more subjects without the specified biological state; a second memory unit for querying a known biomolecule data aggregator, wherein the known biomolecule data aggregator comprises data pertaining to known biomolecules associated with the specified biological state; a first computer executable instruction for building a trained classification model by extracting a first feature of the first biomolecule sampling data and a second feature of the second biomolecule sampling data, wherein the trained classification model of the first feature and the second feature identifies one or more biomarkers linked to the specified biological state; a second computer executable instruction for processing the trained classification model against the known biomolecule data aggregator and assigning a classification weight to all biomolecules, wherein said processing and assigning identifies one or more biomarkers linked to the specified biological state, wherein the one or more biomarkers confirms the specified biological state, wherein the one or more biomarkers is present in a low or previously non-recorded concentration in the first complex biological sampling data; a plurality of nodes connected to each other, each node comprising a computer server, including one or more processors for executing the first computer executable instruction and the second computer executable instruction; network connections to the plurality of nodes; and a communication bus between the computer server, the first memory unit, and the second memory unit.

In some embodiments, the computer-implemented system as described herein further comprises a third computer executable instruction for generating a report of the presence or absence of the specified biological state in a subject. In some embodiments, said report comprises a recommended treatment for a disease management. In some embodiments, the computer-implemented system as described herein further comprises a user interface configured to communicate or display said report to a user. In some embodiments, the computer-implemented system as described herein further catalogs the surface-activity relationship between biomarkers and the particle that captured them to output a Corona Knowledge Map (CKM).

Provided herein is a system comprising a non-transitory computer readable storage medium encoded with a computer program, including instructions executable by a processor, to create an application applying machine learning to a plurality of sample data to rationally design a plurality of features of a particle comprising a corona. The system can comprise a software module applying a machine learning detection structure to the plurality of sample data, the detection structure employing machine learning to screen surface-activity relationships in the plurality of sample data to identify a feature and classify the feature. The feature can be a particle binding region of the biomarker. The system can comprise a software module automatically generating a report comprising the surface-activity relationships of a sample from which said sample data was derived. The report can identify a disease state. The system can comprise a software module automatically generating a report comprising the plurality of features of the particle comprising a corona. The machine learning detection structure can comprise any of the following separately, in series, or in combination: Partial Least Squares, Logistic Regression, Support Vector Classifier, Nearest Neighbor, Random Forest, Naïve Bayes, Ensemble Classifiers, a neural network, a deep network, convolutional neural network, a deep convolutional neural network, a cascaded deep convolutional neural network.

Provided herein is a method of determining an efficacy of a therapeutic treatment of a subject comprising (1) obtaining a plasma sample from a subject before administration of a therapeutic treatment to treat a disease wherein the plasma sample comprises a plurality of plasma particles, (2) obtaining a second plasma sample from the subject after the administration of the therapeutic treatment wherein the second plasma sample comprises a second plurality of plasma particles, (3) isolating the plasma particles from both plasma samples, (4) enriching the biomarkers present in both samples, (5) assaying the enriched biomarkers to generate data, and (6) processing the data. Processing can comprise a trained classifier, wherein said trained classifier assigns a first set of model weights, wᵢ . . . wₙ, for one or more features, fᵢ . . . fₙ, yielding (wᵢ, fᵢ), . . . (wₙ, fₙ) and storing (wᵢ, fᵢ), . . . (wₙ, fₙ) to said first biomarker data to generate weighted first biomarker data, and said trained classifier assigns a second set of model weights, wᵢ . . . wₙ, for one or more features, fᵢ . . . fₙ, yielding (wᵢ, fᵢ), . . . (wₙ, fₙ) and storing (wᵢ, fᵢ), . . . , (wₙ, fₙ) to said second biomarker data to generate weighted second biomarker data. The trained model can be trained using a set of labeled multi-omic data of a plurality of complex biological samples, wherein the labeled multi-omic data set comprises the one or more features fᵢ . . . fₙ corresponding to one or more specified biological states, bᵢ . . . bₙ, wherein the one or more features are proteins. The method can further comprise querying a reference data set for the one or more features of the weighted first biomarker data and the weighted second biomarker data, fᵢ . . . fₙ, to generate a set of scores, sᵢ . . . sₙ, yielding (sᵢ, fᵢ), . . . , (sₙ, fₙ) and storing (sᵢ, fᵢ), . . . , (sₙ, fₙ). The reference data set can be a public database. The method can also comprise combining at least (wᵢ, fᵢ), . . . , (wₙ, fₙ) and (sᵢ, fᵢ), . . . , (sₙ, fₙ) to generate (wᵢ, sᵢ), . . . , (wₙ, sₙ) and selecting a subset of (wᵢ, sᵢ), . . . , (wₙ, sₙ) to generate a first phenotype classification and a second phenotype classification. The method can also comprise determining the efficacy of said therapeutic treatment by comparing said first phenotype classification with said second phenotype classification. The phenotype classification can be a disease state prior to treatment and the second phenotype classification can be a partial response to treatment. In some aspects, at least one particle of the first plurality of plasma particles is a nanoparticle. In some aspects, at least one particle of the second plurality of plasma particles is a nanoparticle. In some aspects, at least one particle of the first isolated plurality of plasma particles is a nanoparticle. In some aspects, at least one particle of the second isolated plurality of plasma particles is a nanoparticle.

Provided herein is a method of determining a concentration of a biomarker in a plasma sample comprising obtaining a reference data set comprising plasma samples with a known biomarker concentration, dispersing particles in each of the reference samples, isolating the particles from the samples, enriching the biomarkers captured by the particles, assaying the biomarkers to generate biomarker data, and incorporating the biomarker data within a trained classifier. The trained classifier can assign a concentration to the biomarker data based on the reference data set. The method can be applied to a plasma sample obtained from a subject such that the trained classifier can then be used to query the reference data set to output a biomarker concentration present in a plasma sample from a subject. The reference data set can comprise biomarker concentrations from 1 pg/mL to 100pg/mL. The reference data set can comprise biomarker concentrations from 1 pg/mL to 100 μg/mL.

Provided herein is a method of analyzing a broad range sampling of a plurality of biomolecules comprising assigning an existing knowledge association score to the plurality of biomolecules in a test data set, generating a classification model weight for the plurality of biomolecules based on the existing knowledge association score, and classifying each biomarker into a category indicative of a likelihood of the biomarker playing a role in the specified biological state. The classification categories can be chosen from one of the following: having a significant classification model weight but with little existing knowledge association for the specified biological state, having a significant classification model weight with well-known existing knowledge association for the specified biological state, having a weak classification model weight with little existing knowledge association for the specified biological state, or having a weak classification model weight with well-known existing knowledge association for the specified biological state. Biomarkers classified as having a significant classification model weight but with little existing knowledge association for the specified biological state can be further classified as novel biomarkers associated with the specified biological state. In some aspects, at least one particle of the plurality is a nanoparticle. In some aspects, the plurality of particles comprises nanoparticles. In some aspects, the plurality of particles is a plurality of nanoparticles.

### BRIEF DESCRIPTION OF THE DRAWINGS

The features of the present disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is an exemplary computer system for implementing a method described herein.

FIG. 2 is a plot of the trained classification model. The lower right quadrant represents proteins that have a significant weight in the classification but with little existing knowledge associating the proteins to the specified disease. Therefore, these proteins are suitable candidates for novel biomarkers. The upper left quadrant represents proteins that have a large body of evidence linking them to the disease but weak classification weights. These proteins could represent either a common mechanism or set of mechanisms for multiple diseases which the classification weights cannot differentiate, or alternatively, could represent a weak role in the classification of the disease. The upper right and lower left quadrants help support the validity of the classification model.

FIG. 3 is a bar graph of classification model weight, represents all 850+ proteins detected in the broad range proteome sampling, ordered in descending classification model weight. The top plot represents the top 50 proteins, and the bottom plot shows all 850+ proteins. The existing knowledge for association to disease is high if that protein is well associated to the disease through existing knowledge. Proteins with low existing knowledge for association to disease represent suitable candidates for potential biomarkers as they represent proteins with a significant classification weight but little existing knowledge.

FIG. 4 is an image of a Corona Knowledge Map (CKM) user interface. The CKM is a relational database which categorizes surface-activity relationships between biomarkers captured and the particle which captured them, for example a corona, in order to optimize particle properties.

### DETAILED DESCRIPTION

The following description and examples illustrate embodiments of the present disclosure in detail.

It is to be understood that the present disclosure is not limited to the particular embodiments described herein and as such can vary. Those of skill in the art will recognize that there are variations and modifications of the present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the disclosure can be described in the context of a single embodiment, the features can also be provided separately or in any suitable combination. Conversely, although the present disclosure can be described herein in the context of separate embodiments for clarity, the present disclosure can also be implemented in a single embodiment.

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

In this application, the use of the singular includes the plural unless specifically stated otherwise. It must be noted that, as used in the specification, the singular forms “a,” “an” and “the” include plural referents unless the context clearly dictates otherwise.

In this application, the use of “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

Furthermore, use of the term “including” as well as other forms, such as “include”, “includes,” and “included,” is not limiting.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the disclosure, and vice versa. Furthermore, compositions of the present disclosure can be used to achieve methods of the present disclosure.

The term “about” in relation to a reference numerical value and its grammatical equivalents as used herein can include the numerical value itself and a range of values plus or minus 10% from that numerical value.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. In another example, the amount “about 10” includes 10 and any amounts from 9 to 11. In yet another example, the term “about” in relation to a reference numerical value can also include a range of values plus or minus 10%, 9%, 8%, 7%, 6%, 5%, 4%, 3%, 2%, or 1% from that value. Alternatively, particularly with respect to biological systems or processes, the term “about” can mean within an order of magnitude, preferably within 5-fold, and more preferably within 2-fold, of a value. Where particular values are described in the application and claims, unless otherwise stated the term “about” meaning within an acceptable error range for the particular value should be assumed.

The term “biomolecule” refers to any molecule or biological component that can be produced by, or is present in, a biological organism. Non-limiting examples of biomolecules include proteins, polypeptides, polysaccharides, a sugar, a lipid, a lipoprotein, a metabolite, an oligonucleotide, a nucleic acid (DNA, RNA, micro RNA, plasmid, single stranded nucleic acid, double stranded nucleic acid), metabolome, as well as small molecules such as primary metabolites, secondary metabolites, and other natural products, or any combination thereof. In some embodiments, the biomolecule is selected from the group of proteins, nucleic acids, lipids, and metabolomes.

As used herein, the term “sensor element” refers to elements that are able to bind to a plurality of biomolecules when in contact with a sample. The term “plurality of sensor elements” refers to more than one, for example, at least two sensor elements. In some embodiments, the plurality of sensor elements includes at least two sensor elements to at least 1000 sensor elements, preferably about two sensor elements to about 100 sensor elements. In suitable embodiments, the array comprises at least two to at least 100 sensor elements, alternatively at least two to at least 50 sensor elements, alternatively at least 2 to 30 sensor elements, alternatively at least 2 to 20 sensor elements, alternatively at least 2 to 10 sensor elements, alternatively at least 3 to at least 50 sensor elements, alternatively at least 3 to at least 30 sensor elements, alternatively at least 3 to at least 20 sensor elements, alternatively at least 3 to at least 10 sensor elements, alternatively at least 4 to at least 50 sensor elements, alternatively at least 4 to at least 30 sensor elements, alternatively at least 4 to at least 20 sensor elements, alternatively at least 4 to at least 10 sensor elements, and including any number of sensor elements contemplated in between (e.g., at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 60, 70, 80, 90, 100, 110, 120, 130, 140, 150, 160, 170, 180, 190, 200, 225, 250, 300, 350, 400, 450, 500, 550, 600, 650, 700, 750, 800, etc.). In some embodiments, the sensor array comprises at least 6 sensor elements to at least 20 sensor elements, alternatively at least 6 sensor elements to at least 10 sensor elements.

As used herein, the term “biomolecule corona” refers to the plurality of different biomolecules that are able to bind to a sensor element. The term “biomolecule corona” encompasses “protein corona” which is a term used in the art to refer to the proteins, lipids and other plasma components that bind nanoparticles when they come into contact with biological samples or biological system. For use herein, the term “biomolecule corona” also encompasses both the soft and hard protein corona as referred to in the art, see, e.g., Milani et al. “Reversible versus Irreversible Binding of Transferring to Polystyrene Nanoparticles: Soft and Hard Corona” ACS NANO, 2012, 6(3), pp. 2532-2541; Mirshafiee et al. “Impact of protein pre-coating on the protein corona composition and nanoparticle cellular uptake” Biomaterials vol. 75, January 2016 pp. 295-304, Mahmoudi et al. “Emerging understanding of the protein corona at the nanobio interfaces” Nanotoday 11(6) December 2016, pp. 817-832, and Mahmoudi et al. “Protein-Nanoparticle Interactions: Opportunities and Challenges” Chem. Rev., 2011, 111(9), pp. 5610-5637, the contents of which are incorporated by reference in their entireties. As described in the art, adsorption curve shows the build-up of a strongly bound monolayer up to the point of monolayer saturation (at a geometrically defined proteinto-NP ratio), beyond which a secondary, weakly bound layer is formed. While the first layer is irreversibly bound (hard corona), the secondary layer (soft corona) exhibits dynamic exchange. Proteins that adsorb with high affinity form what is known as the “hard” corona, consisting of tightly bound proteins that do not readily desorb, and proteins that adsorb with low affinity form the “soft” corona, consisting of loosely bound proteins. Soft and hard corona can also be defined based on their exchange times. Hard corona may show much larger exchange times in the order of several hours. See, e.g., M. Rahman et al. Protein-Nanoparticle Interactions, Spring Series in Biophysics 15, 2013, incorporated by reference in its entirety.

The term “biomolecule corona signature” refers to the composition, signature or pattern of different biomolecules that are bound to each separate sensor element. The signature not only refers to the different biomolecules but also the differences in the amount, level or quantity of the biomolecule bound to the sensor element, or differences in the conformational state of the biomolecule that is bound to the sensor element. It is contemplated that the biomolecule corona signatures of each sensor elements may contain some of the same biomolecules, may contain distinct biomolecules with regard to the other sensor elements, and/or may differ in level or quantity, type or confirmation of the biomolecule. The biomolecule corona signature may depend on not only the physiocochemical properties of the sensor element, but also the nature of the sample and the duration of exposure. In some cases, the biomolecule corona signature is a protein corona signature. In another case, the biomolecule corona signature is a polysaccharide corona signature. In yet another case, the biomolecule corona signature is a metabolite corona signature. In some cases, the biomolecule corona signature is a lipidomic corona signature. In some embodiments, the biomolecule corona signature comprises the biomolecules found in a soft corona and a hard corona. In some embodiments, the soft corona is a soft protein corona. In some embodiments, the hard corona is a hard protein corona.

“Polypeptide(s)”, “peptide(s)” and their grammatical equivalents as used herein refer to a polymer of amino acid residues. Polypeptides can comprise D-amino acids, L-amino acids, and non-natural amino acids, or any combination thereof. A “mature protein” is a protein which is full-length and which, optionally, includes glycosylation or other modifications (e.g., post-translational modification) typical for the protein in a given cellular environment. A protein can be a monomer, a homodimer or a heteromultimer of polypeptides. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The term “lipid(s)” includes a variety of insoluble biomolecules, such as neutral fats, oils, and steroids. Lipids include simple lipids, triglycerides, eicosanoids, complex lipids, phospholipids, steroids, cholesterol, and lipid-related molecules. Simple lipids contain only two types of components, i.e., fatty acids and alcohols. Non-limiting examples of simple lipids include triglycerides, triacylglycerol, diglycerides, and monoglycerides. Fatty acids are long chains of carbon and hydrogen each with a carboxyl acid functional group (—COOH) at one end. The chain length varies but most fatty acids possess even number of carbon atoms with sixteen or eighteen carbon fatty acids as the most common. Fatty acids can be saturated, unsaturated, monounsaturated, or polyunsaturated. Triacylglycerols or triglycerides form when glycerol links to three fatty acids, of which can be different chain lengths. Eicosanoids are modified fatty acids or lipid-related molecules produced by slight alterations in the fatty acid chain of arachidonic acid. Non-limiting examples of eicosanoids can include prostaglandins, thromboxanes, leukotrienes, and lipoxins. Complex lipids can comprise fatty acids, glycerol, and an alcohol besides glycerol, a carbohydrate and a phosphate group. Non-limiting examples of complex lipids include phospholipids and steroids. Phospholipids are phosphate containing lipid molecules. Steroids are a class of lipid-related molecules derived from cholesterol. Non-limiting examples of steroids include cholesterol, testosterone, estrogen and cortisol.

“Polynucleotide” or “oligonucleotide” as used herein refers to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. When a polynucleotide such as an oligonucleotide is represented by a sequence of letters, it is understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T can be used to refer to the bases themselves, to nucleosides, or to nucleotides comprising the bases, as is standard in the art. DNA (deoxyribonucleic acid) is a chain of nucleotides comprising 4 types of nucleotides; A (adenine), T (thymine), C (cytosine), and G (guanine), and that RNA (ribonucleic acid) comprising 4 types of nucleotides; A, U (uracil), G, and C. DNA and RNA can also comprise synthetic nucleotides or chemically modified nucleotides. Certain pairs of nucleotides specifically bind to one another in a complementary fashion (called complementary base pairing). That is, adenine (A) can pair with thymine (T) (in the case of RNA, however, adenine (A) can pair with uracil (U)), and cytosine (C) can pair with guanine (G). When a first nucleic acid strand binds to a second nucleic acid strand made up of nucleotides that are complementary to those in the first strand, the two strands bind to form a double strand.

As used herein, the terms “marker”, “biomarker” (or fragment thereof) and their synonyms, which are used interchangeably, refer to molecules that can be evaluated in a sample and are associated with a specified biological state. For example, markers include genes, expressed genes or their products (e.g., proteins) or autoantibodies to those proteins that can be detected from human samples, such as blood, serum, solid tissue, and the like, that is associated with a specified biological state. Such biomarkers include, but are not limited to, biomolecules comprising polynucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The biomarker can also refer to a portion of a polypeptide (parent) sequence that comprises at least 3 consecutive amino acid residues, at least 10 consecutive amino acid residues, at least 15 consecutive amino acid residues or more, and retains a biological activity and/or some functional characteristics of the parent polypeptide, e.g. antigenicity or structural domain characteristics. The biomarkers refer to both disease biomolecules present on or in diseased cells or those that have been shed from the diseased cells into bodily fluids such as blood or serum. The biomarkers can also refer to biomolecules, including autoantibodies produced by the body to those disease biomolecules. Biomarkers can include any biological substance indicative of the presence of disease, including but not limited to, genetic, epigenetic, proteomic, glycomic or imaging biomarkers, in diseases such as cancer, immunological disorders, neurological disorders, endocrine disorders, metabolic disorders, cardiac diseases. Biomarkers can include molecules secreted by diseased cells and/or other cells, including gene, gene expression, and protein-based products (tumor markers or antigens, cell free DNA, mRNA, etc.)

By “fragment” is meant a portion of a polypeptide or nucleic acid molecule. This portion contains, preferably, at least 1%, 5%, 10%, 20%, 30%, 40%, 50%, 55%, 60%, 65%, 70%, 75%, 80%, 85%, 90%, 91%, 92%, 93%, 94%, 95%, 96%, 97%, 98%, 99%, or 99.9% of the entire length of the reference nucleic acid molecule or polypeptide. A fragment can contain at least 3, 5, 10, 15, 20, 25, 30, 35, 40, 45, 50, 55, 60, 65, 70, 75, 80, 85, 90, 95, 100, 200, 300, 400, 500, 600, 700, 800, 900, 1000, or greater number of nucleotides or amino acids.

The term “isolated” and its grammatical equivalents as used herein refer to the removal of a biomolecule from its natural environment. The term “purified” and its grammatical equivalents as used herein refer to a molecule or composition, whether removed from nature (including genomic DNA and mRNA) or synthesized (including cDNA) and/or amplified under laboratory conditions, that has been increased in purity, wherein “purity” is a relative term, not “absolute purity.” The term “substantially purified” and its grammatical equivalents as used herein refer to a nucleic acid sequence, polypeptide, protein or other compound which is essentially free, i.e., is more than about 50% free of, more than about 60% free of, more than about 70% free of, more than about 80% free of, more than about 90% free of, the polynucleotides, proteins, polypeptides and other molecules that the nucleic acid, polypeptide, protein or other compound is naturally associated with.

By “reference” is meant a standard of comparison.

A “specified biological state” can mean, but is not limited to, a concentration of a biomolecule, a disease state, a poor clinical outcome, a good clinical outcome, a phenotype, a high risk of disease, a low risk of disease, a complete response to a treatment, a partial response to a treatment, a stable disease state, or a non-response to a treatment.

Disease, condition, and disorder are used interchangeably herein.

The term “proliferative disease” as referred to herein refers to a unifying concept in which excessive proliferation of cells and/or turnover of cellular matrix contributes significantly to the pathogenesis of the disease, including cancer.

By “neoplasia” is meant any disease that is caused by or results in inappropriately high levels of cell division, inappropriately low levels of apoptosis, or both. In some embodiments, neoplasia is cancer or tumor.

The term “cancer” and “tumor” as used herein interchangeably and are meant to encompass any cancer, neoplastic and preneoplastic disease that is characterized by abnormal growth of cells. A tumor can be cancerous or benign. A benign tumor means the tumor can grow but does not spread. A cancerous tumor is malignant, meaning it can grow and spread (metastasize) to other parts of the body. Non-limiting examples of cancer include lung cancer, pancreas cancer, myeloma, myeloid leukemia, meningioma, glioblastoma, breast cancer, esophageal squamous cell carcinoma, gastric adenocarcinoma, prostate cancer, bladder cancer, ovarian cancer, thyroid cancer, neuroendocrine cancer, colon carcinoma, ovarian cancer, head and neck cancer, Hodgkin's Disease, non-Hodgkin's lymphomas, rectum cancer, urinary cancers, uterine cancers, oral cancers, skin cancers, stomach cancer, brain tumors, liver cancer, laryngeal cancer, esophageal cancer, mammary tumors, fibrosarcoma, myxosarcoma, liposarcoma, chondrosarcoma, osteogenic sarcoma, chordoma, angiosarcoma, endotheliosarcoma, Ewing's sarcoma, squamous cell carcinoma, basal cell carcinoma, adenocarcinoma, sweat gland carcinoma, sebaceous gland carcinoma, papillary carcinoma, papillary adenocarcinomas, cystandeocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, endometrial cancer, lung carcinoma, small cell lung carcinoma, bladder carcinoma, epithelial carcinoma, glioblastomas, neuronomas, craniopharingiomas, schwannomas, glioma, astrocytoma, meningioma, melanoma, neuroblastoma, retinoblastoma, leukemias and lymphomas, acute lymphocytic leukemia and acute myelocytic polycythemia vera, multiple myeloma, Waldenstrom's macroglobulinemia, and heavy chain disease, acute nonlymphocytic leukemias, chronic lymphocytic leukemia, chronic myelogenous leukemia, childhood-null acute lymphoid leukemia (ALL), thymic ALL, B-cell ALL, acute megakaryocytic leukemia, Burkitt's lymphoma, and T cell leukemia, small and large non-small cell lung carcinoma, acute granulocytic leukemia, germ cell tumors, endometrial cancer, gastric cancer, hairy cell leukemia, thyroid cancer and other cancers known in the art. In a preferred embodiment, the cancer is selected from the group consisting of lung cancer, pancreas cancer, myeloma, myeloid leukemia, meningioma, glioblastoma, breast cancer, esophageal squamous cell carcinoma, gastric adenocarcinoma, prostate cancer, bladder cancer, ovarian cancer, thyroid cancer, and neuroendocrine cancer.

As used herein, the term “sample” refers to a biological sample obtained or derived from a subject. In some embodiments, the sample is a complex biological sample without any prior depletion of a biomolecule (e.g., protein). In some embodiments, a sample comprises nucleic acids representing all or substantially of the nucleic acid sequences found in a subject. In some embodiments, a sample comprises polypeptides or a set of amino acid sequences representing all or substantially of the polypeptide sequences found in a subject. In some embodiments, a sample comprises biological tissue or fluid. Suitable biological samples include, but are not limited to, blood, blood cells, serums, ascites, tissue or fine needle biopsy samples, cell-containing body fluids, lung lavage, cell lysates, bone marrow, sputum, saliva, urine, amniotic fluid, cerebrospinal fluids, tears, semen, tissue biopsy specimens, surgical specimens, other body fluids, secretions, and/or excretions, and/or cells therefrom. Such examples are not however to be construed as limiting the sample types applicable to the present disclosure. The term “tissue” is intended to include intact cells, blood, blood preparations such as plasma and serum, bones, joints, cartilage, neuronal tissue (brain, spinal cord and neurons), muscles, smooth muscles, and organs. In some embodiments, the biological fluids are prepared by methods and kits known in the art. For example, some biological samples (e.g. menstrual blood, blood samples, semen, etc.) can first be centrifuged at low speed to remove cell debris, blood clots and other cellular components that can interfere with the array. In other embodiments, for example, tissue specimens can be processed, e.g., tissue samples can be minced or homogenized, treated with enzymes to break up the tissue and/or centrifuged to remove cellular debris allowing for the assaying and extraction of the biomolecules within the tissue samples. Suitable methods of isolating and/or properly preparing and storing samples are known in the art, and can include, but are not limited, the addition of an anti-coagulant agent.

The terms “complex sample” or “complex biological sample” refer to any biological sample comprising a plurality of any suitable organic molecules (e.g., proteins, polynucleotides, lipids, metabolites), inorganic molecules, submicroscopic agents (e.g., phage). Non-limiting exemplary components of a complex sample include nucleic acid molecules (e.g., nucleotides, oligonucleotides, polynucleotides, DNA, RNA DNA aptamers, RNA aptamers), amino acids, peptides, proteins (native or recombinant), peptide aptamers, antibodies, plasmids, phages, microorganisms, cells, antibodies, organelles, cofactors, and metal ions. The complex sample can be from, but not limited to, body fluids, whole blood, plasma, serum, cerebral spinal fluid (CSF), urine, sweat, saliva, tears, pulmonary secretions, breast aspirate, prostate fluid, seminal fluid, stool, cervical scraping, cysts, amniotic fluid, intraocular fluid, mucous, moisture in breath, animal tissue, cell lysate, tumor tissue, hair, skin, buccal scraping, nail, bone marrow, cartilage, prion, bone powder, ear wax, tumor samples (e.g., fresh, frozen, or paraffin-embedded samples), or any combination thereof.

The term “multi-omic(s)” or “multiomic(s)” refers to an analytical approach for analyzing biomolecules at a large scale, wherein the data sets are multiple omes, such as proteome, genome, transcriptome, lipidome, and metabolome. Non-limiting exemplary multi-omic data includes proteomic data, genomic data, lipidomic data, glycomic data, transcriptomic data, or metabolomics data.

The terms “individual,” “subject,” and “patient” are used interchangeably herein irrespective of whether the subject has or is currently undergoing any form of treatment. In some embodiments, the subject is diagnosed with or suspected of having or developing a disease or disorder. In some embodiments, the disease is cancer. In some embodiments, the subject is in cancer remission. As used herein, the term “subject” generally refers to any vertebrate, including, but not limited to a mammal. Examples of mammals including primates, including simians and humans, equines (e.g., horses), canines (e.g., dogs), felines, various domesticated livestock (e.g., ungulates, such as swine, pigs, goats, sheep, and the like), as well as domesticated pets (e.g., cats, hamsters, mice, and guinea pigs). In some embodiments, the subject is a human. Exemplary human patients can be male and/or female. “Patient in need thereof” or “subject in need thereof” is referred to herein as a patient diagnosed with or suspected of having a disease or disorder, for instance, but not restricted to cancer.

“Administering” is referred to herein as providing one or more compositions described herein to a patient or a subject. By way of example and not limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by the oral route. Additionally, administration can also be, but not limited to, oral administration, mucosal administration, inhalational administration, ocular administration, transdermal administration, rectal administration, intracystic administration, enteral administration, parenteral administration, surgical deposition of a bolus or pellet of cells, or positioning of a medical device.

The terms “treat,” “treated,” “treating,” “treatment,” and their grammatical equivalents are meant to refer to reducing or ameliorating a disorder and/or symptoms associated therewith (e.g., a cancer). “Treating” can refer to administration of the therapy to a subject after the onset, or suspected onset, of a cancer. “Treating” includes the concepts of “alleviating”, which refers to lessening the frequency of occurrence or recurrence, or the severity, of any symptoms or other ill effects related to e.g., a cancer and/or the side effects associated with e.g., cancer therapy. The term “treating” can also encompass the concept of “managing” which refers to reducing the severity of a particular disease or disorder in a patient or delaying its recurrence, e.g., lengthening the period of remission in a patient who had suffered from the disease. It is appreciated that, although not precluded, treating a disorder or condition does not require that the disorder, condition, or symptoms associated therewith be completely eliminated. In embodiments, the effect is therapeutic, i.e., the effect partially or completely cures a disease and/or adverse symptom attributable to the disease.

The term “therapeutic effect” refers to some extent of relief of one or more of the symptoms of a disorder (e.g., a neoplasia or tumor) or its associated pathology.

The term “therapeutically effective amount”, “therapeutic amount” or its grammatical equivalents as used herein refers to an amount of an agent which is effective, upon single or multiple dose administration to the cell or subject, in prolonging the survivability of the patient with such a disorder, reducing one or more signs or symptoms of the disorder, preventing or delaying, and the like beyond that expected in the absence of such treatment. “Therapeutically effective amount” is intended to qualify the amount required to achieve a therapeutic effect. The therapeutically effective amount can vary according to factors such as the disease state, age, sex, and weight of the individual, and can be determined by a physician with consideration of individual differences in e.g., age, weight, tumor size, extent of infection or metastasis, and condition of the patient (subject).

### Overview

In training classifiers against complex biomolecule sampling, the classification model weights are dependent on the dataset and are relative within the dataset. For example, if a feature (e.g., protein) is removed from a data set, the weights of the other features are recalculated during retraining. The methods disclosed herein provide consistency of important features across many independent datasets by focusing on the subset of the important features consistent across different datasets. The methods provided herein identify the subset currently not related to known knowledge as a novel biomarker.

The present disclosure relates to a method of broad dynamic range sampling of biomolecules in a complex biological sample. In an embodiment, the method relates to a proteome sampling without prior protein depletion. In an embodiment, the method relates to a proteome sampling before prior protein depletion. In an embodiment, the method relates to a proteome sampling after prior protein depletion. In another embodiment, the method of proteome sampling is independent of direct plasma protein concentration, enabling accurate clustering algorithms to generate protein biomarkers. In an embodiment, the method relates to analyzing multiple samplings such that a relational database, a Corona Knowledge Map (CKM), is produced which can be used to rationally design particles, such as a corona, based on the surface-activity relationship of biomarkers to the particle, such as a corona, that captured them.

There are several public searchable metabolome database and proteome database known in the art. The present methods of the disclosure can be used with such public databases. Examples of public databases include but are not limited to Open Targets (opentargets.org), Gene Ontology Consortium (geneontology.org), Plasma Proteome Database (plasmaproteomedatabase.org), METLIN (metlin.scripps.edu), Human Metabolome Database (hmdb.ca), Kyoto Encyclopedia of Genes and Genomes (genome.jp/kegg/), Biological Magnetic Resonance Bank (bmrb.wisc.edu/metabolomics/), Proteomics Identifications (PRIDE) (ebi.ac.uk/pride), ProteomicsDB (proteomicsdb.org), or Biological Magnetic Resonance Bank (bmrb.wisc.edu/metabolomics/). This existing public knowledge base can be leveraged individually or by using aggregators such as Open Targets, Gene Ontology Consortium, and similar commercial offerings. In addition to public knowledge bases, the methods herein can also be used with proprietary databases. For example, the Corona Knowledge Map (CKM) is a relational database to rationally design particles based on the analysis of surface-activity relationships (e.g., referencing data from a protein structure database and/or a protein-ligand co-crystal structure database) between the biomarkers captured and the particle which captured them.

Provided herein is a new approach to broadly sample the proteome without, before and after protein depletion and/or independent of direct plasma protein concentration. The present disclosure can identify proteins that are linked to a specified biological state (e.g., a disease) but which are present in very low or non-recorded concentrations in the plasma, serum, whole blood, amniotic fluid, cerebral spinal fluid, urine, saliva, tears, feces according to the Plasma Proteome Database and other databases. Non-limiting examples of a specified biological state is a disease state, a poor clinical outcome, a good clinical outcome, a high risk of disease, a low risk of disease, a complete response to a treatment, a partial response to a treatment, a stable disease state, or a non-response to a treatment. Non-limiting exemplary proteins labeled with disease is provided in Table 1.

“Biological state” encompasses any biological characteristic of a subject which can be manifested in a biological sample as defined herein. A biological state can be detected using the methods disclosed herein where two subjects who differ in the biological state manifest those differences in the composition of a sample. For example, a biological state includes a disease state of a subject. A disease state can be detected when the disease state gives rise to changes in the molecular composition (e.g., level of one or more proteins) of a sample of a subject expressing the disease state relative to a sample of a subject not having the disease state (e.g., where the biological state is a healthy state or non-disease state).

Another example of a biological state is a level of responsiveness of a subject to a particular therapeutic treatment (e.g. administration of one or a combination of drugs or pharmaceuticals). In some embodiments, a biological state is responsiveness (e.g., with respect to a particular threshold of analysis) of a subject to a particular drug. In another embodiment, a biological state is non-responsiveness (e.g., with respect to a particular threshold of analysis) of a subject to a particular drug. In some embodiments, the level of responsiveness of a subject to a drug (i.e., biological state of responsiveness to the drug or biological state of non-responsiveness to the drug) is associated with factors such as variability in metabolism or pharmacokinetics of the drug between subjects.

Another example of a biological state is the level of immune response exhibited by a subject. In some embodiments, the biological state can be increased immune response. In other embodiments, the biological state can be decreased immune response. Immune response can differ between subjects as a result of a number of variables. For example, immune response can differ between subjects as a result of differing exposure to an exogenously introduced antigen (e.g. associated with a virus or bacteria), as a result of differences in their susceptibility to an autoimmune disease or disorder, or secondarily as a result of a response to other biological states in a subject (e.g., disease states such as cancer).

The term “disease state” for a subject as used herein refers to the ability of sensor array of the present technology to be able to differentiate between the different states of a disease within a subject. This term encompasses a predisease state or precursor state of a disease or disorder (a state in which the subject may not have any outward signs or symptoms of the disease or disorder but will develop the disease or disorder in the future) and a disease state in which the subject has a stage of the disease or disorder (e.g., an early, intermediate or late stage of the disease or disorder). In other words, the disease state is a spectrum that encompasses a continuum regarding the health of a subject with respect to a disease or disorder.

The disease state also includes a precursor state of a disease or disorder. This precursor state is a state in which the subject does not have any outward signs or symptoms of the disease or disorder (although there may be submacro changes within the biomolecules of the subject found in their blood or other biological fluids) but will develop the disease or disorder in the future.

In some embodiments, the methods of the present technology include comparing the protein fingerprint of the sample to a panel of protein fingerprints associated with a plurality of diseases and/or a plurality of disease states to determine if the sample indicates a disease and/or disease state. For example, samples can be collected from a population of subjects over time. Once the subjects develop a disease or disorder, the present invention allows for the ability to characterize and detect the changes in biomolecule fingerprints over time in the subject by comparing the biomolecule fingerprint of the sample from the same subject before they have developed a disease to the biomolecule fingerprint of the subject after they have developed the disease. In some embodiments, samples can be taken from cohorts of patients who all develop the same disease, allowing for analysis and characterization of the biomolecule fingerprints that are associated with the different stages of the disease for these patients (e.g. from pre-disease to disease states).

Methods of determining a biomolecule fingerprint associated with at least one disease or disorder and/or a disease state are contemplated. The methods comprise the steps of obtaining a sample from at least two subjects diagnosed with the at least one disease or disorder or having the same disease state; contacting each sample with a sensor array described herein to determining a biomolecule fingerprint for each sensor array, and analyzing the fingerprint of the at least two samples to determine a biomolecule fingerprint associated with the at least one disease or disorder and/or disease state.

Provided herein is a computer-implemented method for detecting one or more biomarkers in a multi-omic data set (e.g., proteomic data set). In some embodiments, the computer-implemented method comprises (1) providing a multi-omic data (e.g., proteomic data) generated from one or more complex biological samples obtained from one or more individuals, each individual having one or more specified biological states; (2) applying a trained model to the multi-omic data (e.g., proteome data) to generate one or more classification model weights, wᵢ . . . wₙ, for one or more features, fᵢ . . . fₙ, yielding (wᵢ,fᵢ), . . . , (wₙ,fₙ) and storing (wᵢ,fᵢ), . . . , (wₙ,fₙ); (3) querying a reference data set for the one or more features, fᵢ . . . fₙ, to generate a set of scores, sᵢ . . . sₙ, yielding (sᵢ,fᵢ), . . . , (sₙ,fₙ) and storing (sᵢ,fᵢ), . . . , (sₙ,fₙ); and (4) combining at least (wᵢ,fᵢ), . . . , (wₙ,fₙ) and (sᵢ,fᵢ), . . . , (sₙ,fₙ) to generate (wᵢ,sᵢ), . . . , (wₙ, sₙ) and selecting a subset of (wᵢ,sᵢ), . . . , (wₙ, sₙ) to detect one or more biomarkers.

In some embodiments, the method further comprises obtaining one or more complex biological samples from one or more individuals. In some embodiments, the multi-omic data is proteomic data, genomic data, lipidomic data, glycomic data, transcriptomic data, or metabolomics data. In some embodiments, the one or more complex biological samples are plasma proteome samples. In some cases, the proteome data comprises (i) protein identifiers and (ii) specified biological states for the one or more individuals. The proteome data can be generated by assaying a complex biological sample of an individual of the one or more individuals as describe herein. The trained model can be trained using a set of labeled data of a plurality of complex biological samples, wherein the labeled data set comprises one or more features fᵢ . . . fₙ corresponding to one or more specified biological states bᵢ . . . bₙ, wherein the one or more features are proteins. In some cases, the one or more features represent different proteins. The reference data set can be a database comprising features related to specified biological states by an association score. The set of scores, sᵢ . . . sₙ, can be association scores between the one or more features and the one or more specified biological states. In some cases, the selecting the subject in step (4) comprises filtering (wᵢ,sᵢ), . . . , (wₙ, sₙ) such that w at least meets a first threshold and s at least meets a second threshold such that the one or more biomarkers comprise a subset (wₖ,sₖ) . . . (wₘ,sₘ) of (wᵢ,sᵢ), . . . , (wₙ, sₙ). In some cases, k≥i. In some cases, m≤n. In some cases, the method can further comprise generating an output. The output can correspond to a specified biological state of the one or more specified biological states. In some cases, the one or more complex biological samples are not subjected to protein depletion. In one embodiment, the surface-activity relationships between biological sample biomarker and capture particle, such as a corona, are catalogued into a relational database known as a Corona Knowledge Map (CKM). The CKM can be used to rationally design particles to target potential biomarkers.

### Complex Biomolecule Sampling

The present disclosure relates to generating potential biomarkers present in a low or non-recorded concentration in samples by applying machine learning clustering algorithms to a large disease labeled training set of complex biomolecule data or proteome data (e.g., plasma protein levels) and comparing the classification weights to existing knowledge bases. Techniques to produce broad range sampling of complex biomolecules (e.g., plasma proteome) without a prior depletion and independent of e.g., plasma protein concentration and to generate a large training and test data set of disease labeled biomolecule (e.g., protein) levels across many patient sample are described in International Patent Application PCT/US2017/067013, which is herein incorporated by reference in its entirety.

In some embodiments, classification model weight and importance for each protein in each disease label, the definition depends on the algorithm used. The classification model weight of Table 1 is generated using Random Forest. Random Forest allows estimation of the classification model weight and importance for each protein by a number of methods, one being removing or perturbing the values. The average of the resulting changes in classification error provides an indication of weight. The classification model weights are dependent on the dataset and are relative within the dataset. For example, if a feature (protein) is removed from a data set, the weights of the other features are recalculated when the model is retrained. For example, if the same algorithm is used on a similar but a new set of data, the weight can be different. The methods disclosed herein provide consistency of important features across many independent datasets by focusing on the subset of the important features consistent across different datasets. The methods provided herein identify the subset currently not related to known knowledge as a novel biomarker.

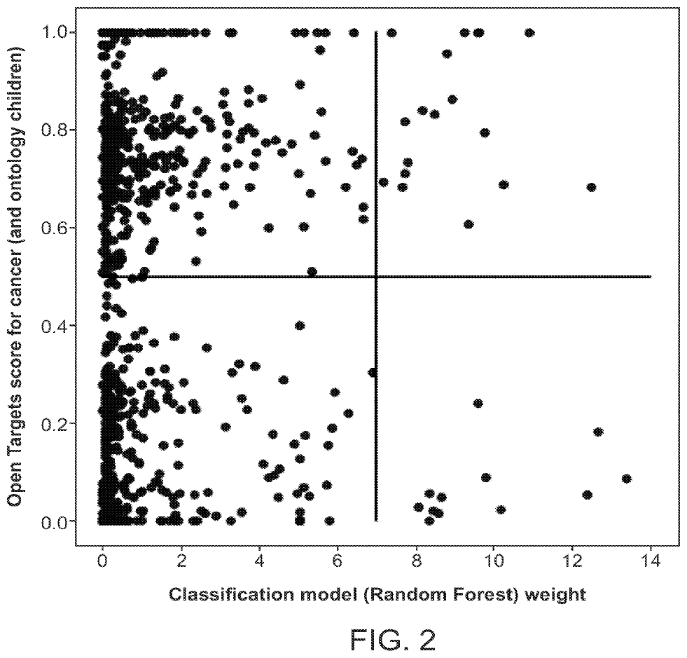

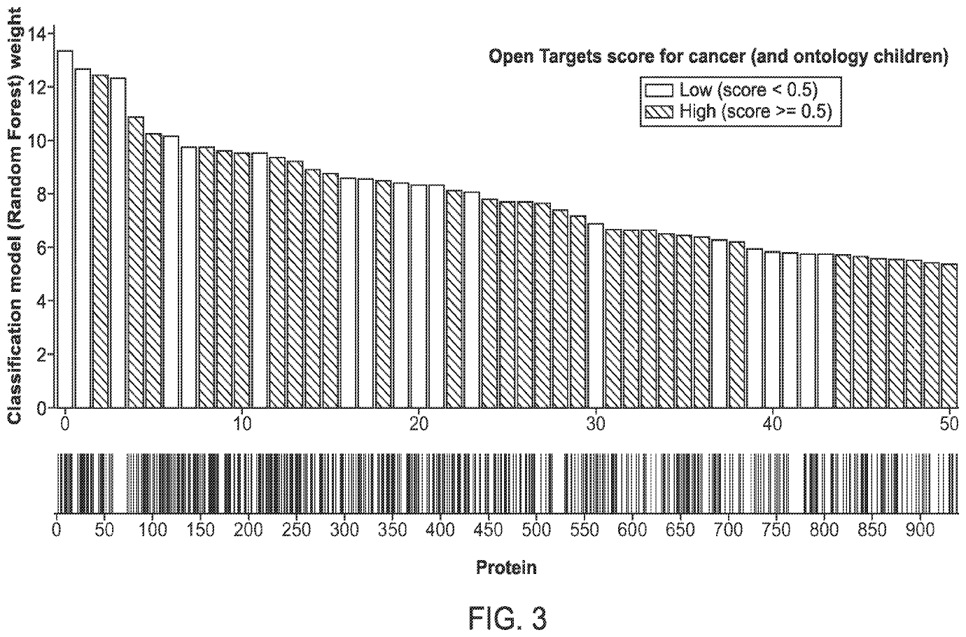

Machine learning classification algorithms such as Partial Least Squares, Logistic Regression, Support Vector Classifier, Nearest Neighbor, Random Forest can be applied to the disease labeled biomolecules to build a trained classification model with both a high sensitivity and specificity. The features of individual biomolecules are extracted by inspecting the trained classification model and their associated classification weights and store as a set of data. Another data set is created by querying data aggregators such as Open Targets, Gene Ontology Consortium and commercial options for all known biomolecules (e.g., proteins or expressing genes) connected with the labeled diseases and their respective association score. The classification strength of the biomolecule (e.g., protein) to its strength of associations in public and private databases to the labeled diseases is compared for the set of potential biomarkers. Exemplary analysis of the trained classification model of protein proteome is shown in FIG. 2 and FIG. 3. The lower right quadrant of FIG. 2 represents proteins that have a significant weight in the classification but with little existing knowledge associating the proteins to the specified disease. Therefore, these proteins are suitable candidates for novel biomarkers. The upper left quadrant of FIG. 2 represents proteins that have a large body of evidence linking them to the disease but weak classification weights. These proteins could represent either a common mechanism or set of mechanisms for multiple diseases which the classification weights cannot differentiate, or alternatively, could represent a weak role in the classification of the disease. These exemplary proteins referred to in FIG. 2 are shown in Table 2.

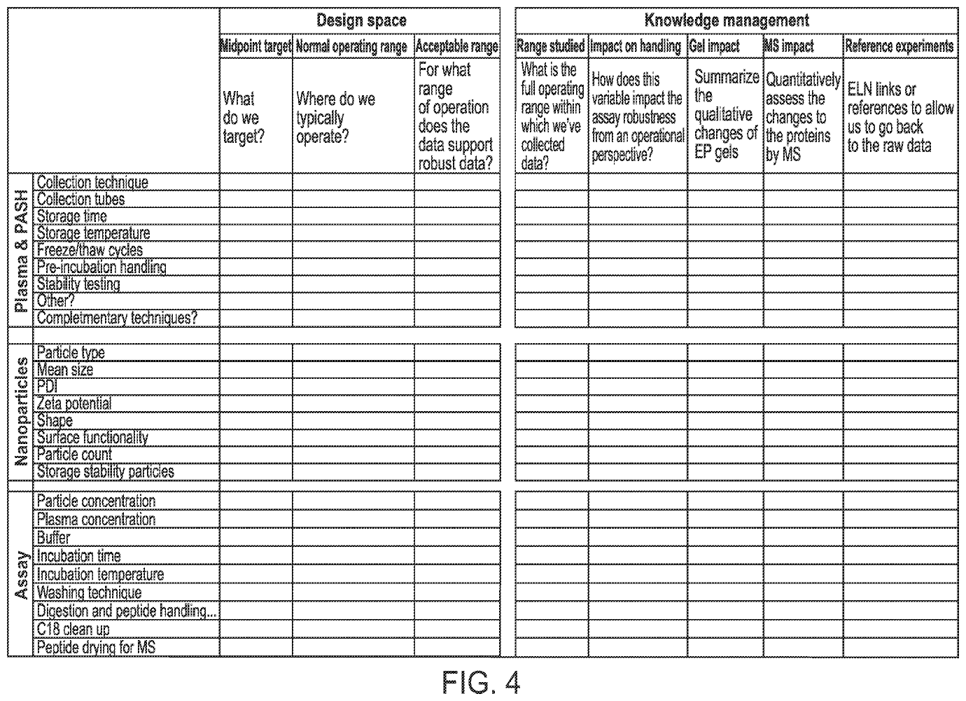

The upper right and lower left quadrants help support the validity of the classification model. FIG. 3 is a bar graph of classification model weight, represents all 850+ proteins detected in the broad range proteome sampling, ordered in descending classification model weight. The top plot represents the top 50 proteins (Table 2), and the bottom plot shows all 850+ proteins. The existing knowledge for association to disease is high if that protein is well associated to the disease through existing knowledge. Proteins with low existing knowledge for association to disease represent suitable candidates for potential biomarkers as they represent proteins with a significant classification weight but little existing knowledge. FIG. 4 is an image of a user interface of a knowledge map wherein information on the surface-activity relationship between captured proteins and the particle which captured them is catalogued such that particles can be rationally designed to target potential biomarker candidates.

The methods of the present disclosure can be used with a complex biological sample. Biomolecules can refer to any molecule or biological component that can be produced by, present in, a biological organism. Non-limiting examples of biomolecules include proteins, polypeptides, polysaccharides, a sugar, a lipid, a lipoprotein, a metabolite, an oligonucleotide, a nucleic acid (DNA, RNA, microRNA, plasmid, single stranded nucleic acid, and double stranded nucleic acid), metabolome, as well as small molecules such as primary metabolites, secondary metabolites, and other natural products, or any combination thereof. In some embodiments, the biomolecule is selected from the group of proteins (e.g., polypeptides or peptides), nucleic acids, lipids, and metabolomes.

In some embodiments, the protein is a mature protein. In some embodiments, the mature protein can include glycosylation or other modification (e.g., post-translational modification). Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, famesylation, geranylation, glypiation, lipoylation and iodination. Polypeptides can comprise D-amino acids, L-amino acids, and non-natural amino acids, or any combination thereof. In some embodiments, the proteins can be a monomer, a homodimer, or a heteromultimer of polypeptides.

In some embodiments, the lipids include a variety of insoluble biomolecules, such as neutral fats, oils, and steroids. Lipids include simple lipids, triglycerides, eicosanoids, complex lipids, phospholipids, steroids, cholesterol, and lipid-related molecules. Simple lipids contain only two types of components, i.e., fatty acids and alcohols. Non-limiting examples of simple lipids include triglycerides, triacylglycerol, diglycerides, and monoglycerides. Fatty acids are long chains of carbon and hydrogen each with a carboxyl acid functional group (—COOH) at one end. The chain length varies but most fatty acids possess even number of carbon atoms with sixteen or eighteen carbon fatty acids as the most common. Fatty acids can be saturated, unsaturated, monounsaturated, or polyunsaturated. Triacylglycerols or triglycerides form when glycerol links to three fatty acids, of which can be different chain lengths. Eicosanoids are modified fatty acids or lipid-related molecules produced by slight alterations in the fatty acid chain of arachidonic acid. Non-limiting examples of eicosanoids can include prostaglandins, thromboxanes, leukotrienes, and lipoxins. Complex lipids can comprise fatty acids, glycerol, and an alcohol besides glycerol, a carbohydrate and a phosphate group. Non-limiting examples of complex lipids include phospholipids and steroids. Phospholipids are phosphate containing lipid molecules. Steroids are a class of lipid-related molecules derived from cholesterol. Non-limiting examples of steroids include cholesterol, testosterone, estrogen and cortisol.

In some embodiments, nucleic acids comprise to a polymeric form of nucleotides or nucleic acids of any length, either ribonucleotides or deoxyribonucleotides. This term refers only to the primary structure of the molecule. Thus, this term includes double and single stranded DNA, triplex DNA, as well as double and single stranded RNA. It also includes modified, for example, by methylation and/or by capping, and unmodified forms of the polynucleotide. When a polynucleotide such as an oligonucleotide is represented by a sequence of letters, it is understood that the nucleotides are in 5′→3′ order from left to right and that “A” denotes deoxyadenosine, “C” denotes deoxycytidine, “G” denotes deoxyguanosine, and “T” denotes thymidine, unless otherwise noted. The letters A, C, G, and T can be used to refer to the bases themselves, to nucleosides, or to nucleotides comprising the bases, as is standard in the art. DNA (deoxyribonucleic acid) is a chain of nucleotides comprising 4 types of nucleotides; A (adenine), T (thymine), C (cytosine), and G (guanine), and that RNA (ribonucleic acid) comprising 4 types of nucleotides; A, U (uracil), G, and C. DNA and RNA can also comprise synthetic nucleotides or chemically modified nucleotides. Certain pairs of nucleotides specifically bind to one another in a complementary fashion (called complementary base pairing). That is, adenine (A) can pair with thymine (T) (in the case of RNA, however, adenine (A) can pair with uracil (U)), and cytosine (C) can pair with guanine (G). When a first nucleic acid strand binds to a second nucleic acid strand made up of nucleotides that are complementary to those in the first strand, the two strands bind to form a double strand.

Biomarkers identified with the methods provided herein can be biomolecules that can be evaluated in a sample and are associated with a specified biological state. The specified biological state can mean, but not limited to, a disease state, a poor clinical outcome, a good clinical outcome, a high risk of disease, a low risk of disease, a complete response to a treatment, a partial response to a treatment, a stable disease state, or a non-response to a treatment. In some embodiments, a disease state refers to cancer prognosis, cancer diagnosis, cancer treatment response, cancer treatment option, post-cancer management, or any combination thereof.

In some embodiments, biomarkers are the molecules that can be evaluated in a sample and are associated with a specified biological state. For example, markers include genes, expressed genes or their products (e.g., proteins) or autoantibodies to those proteins that can be detected from human samples, such as blood, serum, solid tissue, and the like, that is associated with a specified biological state. Such biomarkers include, but are not limited to, biomolecules comprising polynucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The biomarker can also refer to a portion of a polypeptide (parent) sequence that comprises at least 3 consecutive amino acid residues, at least 10 consecutive amino acid residues, at least 15 consecutive amino acid residues or more, and retains a biological activity and/or some functional characteristics of the parent polypeptide, e.g. antigenicity or structural domain characteristics. The biomarkers refer to both disease biomolecules present on or in diseased cells or those that have been shed from the diseased cells into bodily fluids such as blood or serum. The biomarkers can also refer to biomolecules, including autoantibodies produced by the body to those disease biomolecules. Biomarkers can include any biological substance indicative of the presence of disease, including but not limited to, genetic, epigenetic, proteomic, glycomic or imaging biomarkers, in diseases such as cancer, immunological disorders, neurological disorders, endocrine disorders, metabolic disorders, cardiac diseases. Biomarkers can include molecules secreted by diseased cells and/or other cells, including gene, gene expression, and protein-based products (tumor markers or antigens, cell free DNA, mRNA, etc.)

Additionally, the specified biological state herein includes, but not limited to, a diagnosis of cancer at early stage using one or more biomarkers identified by the methods described herein. In other embodiments, the specified biological state herein includes refractory or recurrent malignancies whose growth can be inhibited by targeting one or more biomarkers identified by the methods described herein.

Non-limiting examples of cancers include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies. In other embodiments, cancer can be selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer can be treated by targeting one or more biomarkers identified by the methods described herein include sarcomata (for example, myogenic sarcoma), leukosis, neuroma, melanoma, and lymphoma. In some embodiments, cancer is a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, cancer is a hematological cancer. In some embodiments, a hematological cancer is Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”).

Non-limiting examples of cancers that can be diagnosed early with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke.

The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison's Disease, Adrenal Cancer, Adrenal Disorders, Anaplastic Thyroid Cancer, Cushing's Syndrome, De Quervain's Thyroiditis, Diabetes, Follicular Thyroid Cancer, Gestational Diabetes, Goiters, Graves' Disease, Growth Disorders, Growth Hormone Deficiency, Hashimoto's Thyroiditis, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet's disease, spondyloarthritis, uveitis, systemic lupus erythematosus, and cancer. For example, the arthritis includes rheumatoid arthritis, psoriatic arthritis, osteoarthritis orjuvenile idiopathic arthritis, and the like.

Neurological disorders or neurological diseases are used interchangeably and refer to diseases of the brain, spine and the nerves that connect them. Neurological diseases include, but are not limited to, brain tumors, epilepsy, Parkinson's disease, Alzheimer's disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic's disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immunemediated encephalomyelitis, immune-mediated autonomic neuropathy, myasthenia gravis, autoantibody-associated encephalopathy, and acute disseminated encephalomyelitis.

In some embodiments, biomarkers can include expressed genes or their products (e.g., proteins) or autoantibodies to those proteins that can be detected from human samples, such as body fluids, whole blood, plasma, serum, cerebral spinal fluid (CSF), urine, sweat, saliva, tears, pulmonary secretions, breast aspirate, prostate fluid, seminal fluid, stool, cervical scraping, cysts, amniotic fluid, intraocular fluid, mucous, moisture in breath, animal tissue, cell lysate, tumor tissue, hair, skin, buccal scraping, nail, bone marrow, cartilage, prion, bone powder, ear wax, tumor samples (e.g., fresh, frozen, or paraffin-embedded samples), or any combination thereof, that is associated with a specified biological state. Such biomarkers include, but are not limited to, biomolecules comprising polynucleotides, amino acids, sugars, fatty acids, steroids, metabolites, polypeptides, proteins (such as, but not limited to, antigens and antibodies), carbohydrates, lipids, hormones, antibodies, regions of interest which serve as surrogates for biological molecules, combinations thereof (e.g., glycoproteins, ribonucleoproteins, lipoproteins) and any complexes involving any such biomolecules, such as, but not limited to, a complex formed between an antigen and an autoantibody that binds to an available epitope on said antigen. The biomarker can also refer to a portion of a polypeptide (parent) sequence that comprises at least 3 consecutive amino acid residues, at least 10 consecutive amino acid residues, at least 15 consecutive amino acid residues or more, and retains a biological activity and/or some functional characteristics of the parent polypeptide, e.g. antigenicity or structural domain characteristics. The biomarkers refer to both disease biomolecule present on or in diseased cells or those that have been shed from the diseased cells into bodily fluids such as blood or serum. The biomarkers can also refer to biomolecules, including autoantibodies produced by the body to those disease biomolecules. Biomarkers can include any biological substance indicative of the presence of disease, including but not limited to, genetic, epigenetic, proteomic, glycomic or imaging biomarkers, in diseases such as cancer, immunological disorders, neurological disorders, endocrine disorders, metabolic disorders, or cardiac diseases. Biomarkers can include molecules secreted by diseased cells, including gene, gene expression, and protein-based products (tumor markers or antigens, cell free DNA, mRNA, etc.).

In some embodiments, the biomarker can comprise one or more protein or a fragment of the protein encoded by genes selected from CPN1, FCN3, SAA4, IGHG1, IGHG3, CFHR5, C4B, IGLL5, APOD, SERPINA10, CPN2, FGL1, AHSG, ITIH2, HIST1H4D, C4A, CP, CD5L, CNN2, HRNR, GPLD1, IGKC, MASP2, ITIH1, CFHR1, COLEC10, BIN2, SAA2, ANGPTL6, CFB, TPI1, IGHA2, APOC2, EMILIN1, SBSN, PRG4, PPIF, CFHR2, ORMI, AMYIC, NEXN, CALML5, SERPINA7, IGHM, TUFM, APCS, SLC2A3, TMSB4X, CPQ, and SNCA.

In some embodiments, the biomarker is present in a complex biological sample at about 1 pg/ml, about 2 pg/ml, about 3 pg/ml, about 4 pg/ml, about 5 pg/ml, about 6 pg/ml, about 7 pg/ml, about 8 pg/ml, about 9 pg/ml, about 10 pg/ml, about 20 pg/ml, about 30 pg/ml, about 40 pg/ml, about 50 pg/ml, about 60 pg/ml, about 70 pg/ml, about 80 pg/ml, about 90 pg/ml, or about 100 pg/ml.

In some embodiments, the biomarker is present in a complex biological sample at about 1 pg/ml or more, about 2 pg/ml or more, about 3 pg/ml or more, about 4 pg/ml or more, about 5 pg/ml or more, about 6 pg/ml or more, about 7 pg/ml or more, about 8 pg/ml or more, about 9 pg/ml or more, about 10 pg/ml or more, about 20 pg/ml or more, about 30 pg/ml or more, about 40 pg/ml or more, about 50 pg/ml or more, about 60 pg/ml or more, about 70 pg/ml or more, about 80 pg/ml or more, about 90 pg/ml or more, or about 100 pg/ml or more.

In some embodiments, the biomarker is present in a complex biological sample at about 1 pg/ml or less, about 2 pg/ml or less, about 3 pg/ml or less, about 4 pg/ml or less, about 5 pg/ml or less, about 6 pg/ml or less, about 7 pg/ml or less, about 8 pg/ml or less, about 9 pg/ml or less, about 10 pg/ml or less, about 20 pg/ml or less, about 30 pg/ml or less, about 40 pg/ml or less, about 50 pg/ml or less, about 60 pg/ml or less, about 70 pg/ml or less, about 80 pg/ml or less, about 90 pg/ml or less, or about 100 pg/ml or less.

In some embodiments, the biomarker is present in a complex biological sample at about 0.1 ng/ml, about 0.2 ng/ml, about 0.3 ng/ml, about 0.4 ng/ml, about 0.5 ng/ml, about 0.6 ng/ml, about 0.7 ng/ml, about 0.8 ng/ml, about 0.9 ng/ml, about 1 ng/ml, about 2 ng/ml, about 3 ng/ml, about 4 ng/ml, about 5 ng/ml, about 6 ng/ml, about 7 ng/ml, about 8 ng/ml, about 9 ng/ml, about 10 ng/ml, about 20 ng/ml, about 30 ng/ml, about 40 ng/ml, about 50 ng/ml, about 60 ng/ml, about 70 ng/ml, about 80 ng/ml, about 90 ng/ml, about 100 ng/ml, about 200 ng/ml, about 300 ng/ml, about 400 ng/ml, about 500 ng/ml, about 600 ng/ml, about 700 ng/ml, about 800 ng/ml, about 900 ng/ml, or about 1000 ng/ml.

In some embodiments, the biomarker is present in a complex biological sample at about 0.1 ng/ml or more, about 0.2 ng/ml or more, about 0.3 ng/ml or more, about 0.4 ng/ml or more, about 0.5 ng/ml or more, about 0.6 ng/ml or more, about 0.7 ng/ml or more, about 0.8 ng/ml or more, about 0.9 ng/ml or more, about 1 ng/ml or more, about 2 ng/ml or more, about 3 ng/ml or more, about 4 ng/ml or more, about 5 ng/ml or more, about 6 ng/ml or more, about 7 ng/ml or more, about 8 ng/ml or more, about 9 ng/ml or more, about 10 ng/ml or more, about 20 ng/ml or more, about 30 ng/ml or more, about 40 ng/ml or more, about 50 ng/ml or more, about 60 ng/ml or more, about 70 ng/ml or more, about 80 ng/ml or more, about 90 ng/ml or more, about 100 ng/ml or more, about 200 ng/ml or more, about 300 ng/ml or more, about 400 ng/ml or more, about 500 ng/ml or more, about 600 ng/ml or more, about 700 ng/ml or more, about 800 ng/ml or more, about 900 ng/ml or more, or about 1000 ng/ml or more.

In some embodiments, the biomarker is present in a complex biological sample at about 0.1 ng/ml or less, about 0.2 ng/ml or less, about 0.3 ng/ml or less, about 0.4 ng/ml or less, about 0.5 ng/ml or less, about 0.6 ng/ml or less, about 0.7 ng/ml or less, about 0.8 ng/ml or less, about 0.9 ng/ml or less, about 1 ng/ml or less, about 2 ng/ml or less, about 3 ng/ml or less, about 4 ng/ml or less, about 5 ng/ml or less, about 6 ng/ml or less, about 7 ng/ml or less, about 8 ng/ml or less, about 9 ng/ml or less, about 10 ng/ml or less, about 20 ng/ml or less, about 30 ng/ml or less, about 40 ng/ml or less, about 50 ng/ml or less, about 60 ng/ml or less, about 70 ng/ml or less, about 80 ng/ml or less, about 90 ng/ml or less, about 100 ng/ml or less, about 200 ng/ml or less, about 300 ng/ml or less, about 400 ng/ml or less, about 500 ng/ml or less, about 600 ng/ml or less, about 700 ng/ml or less, about 800 ng/ml or less, about 900 ng/ml or less, or about 1000 ng/ml or less.

In some embodiments, the biomarker is present in a complex biological sample at about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 20 μg/ml, about 30 μg/ml, about 40 μg/ml, about 50 μg/ml, about 60 μg/ml, about 70 μg/ml, about 80 μg/ml, about 90 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, or about 1000 μg/ml.

In some embodiments, the biomarker is present in a complex biological sample at about 1 μg/ml or more, about 2 μg/ml or more, about 3 μg/ml or more, about 4 μg/ml or more, about 5 μg/ml or more, about 6 μg/ml or more, about 7 μg/ml or more, about 8 μg/ml or more, about 9 μg/ml or more, about 10 μg/ml or more, about 20 μg/ml or more, about 30 μg/ml or more, about 40 μg/ml or more, about 50 μg/ml or more, about 60 μg/ml or more, about 70 μg/ml or more, about 80 μg/ml or more, about 90 μg/ml or more, about 100 μg/ml or more, about 200 μg/ml or more, about 300 μg/ml or more, about 400 μg/ml or more, about 500 μg/ml or more, about 600 μg/ml or more, about 700 μg/ml or more, about 800 μg/ml or more, about 900 μg/ml or more, or about 1000 μg/ml or more.

In some embodiments, the biomarker is present in a complex biological sample at about 1 μg/ml or less, about 2 μg/ml or less, about 3 μg/ml or less, about 4 μg/ml or less, about 5 μg/ml or less, about 6 μg/ml or less, about 7 μg/ml or less, about 8 μg/ml or less, about 9 μg/ml or less, about 10 μg/ml or less, about 20 μg/ml or less, about 30 μg/ml or less, about 40 μg/ml or less, about 50 μg/ml or less, about 60 μg/ml or less, about 70 μg/ml or less, about 80 μg/ml or less, about 90 μg/ml or less, about 100 μg/ml or less, about 200 μg/ml or less, about 300 μg/ml or less, about 400 μg/ml or less, about 500 μg/ml or less, about 600 μg/ml or less, about 700 μg/ml or less, about 800 μg/ml or less, about 900 μg/ml or less, or about 1000 μg/ml or less.

In some embodiments, the biomarker present in a complex biological sample is at about 1 mg/ml, about 2 mg/ml, about 3 mg/ml, about 4 mg/ml, about 5 mg/ml, about 6 mg/ml, about 7 mg/ml, about 8 mg/ml, about 9 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 60 mg/ml, about 70 mg/ml, about 80 mg/ml, about 90 mg/ml, about 100 mg/ml, about 200 mg/ml, about 300 mg/ml, about 400 mg/ml, about 500 mg/ml, about 600 mg/ml, about 700 mg/ml, about 800 mg/ml, about 900 mg/ml, or about 1000 mg/ml.

In some embodiments, the biomarker present in a complex biological sample is at about 1 mg/ml or more, about 2 mg/ml or more, about 3 mg/ml or more, about 4 mg/ml or more, about 5 mg/ml or more, about 6 mg/ml or more, about 7 mg/ml or more, about 8 mg/ml or more, about 9 mg/ml or more, about 10 mg/ml or more, about 15 mg/ml or more, about 20 mg/ml or more, about 25 mg/ml or more, about 30 mg/ml or more, about 35 mg/ml or more, about 40 mg/ml or more, about 45 mg/ml or more, about 50 mg/ml or more, about 60 mg/ml or more, about 70 mg/ml or more, about 80 mg/ml or more, about 90 mg/ml or more, about 100 mg/ml or more, about 200 mg/ml or more, about 300 mg/ml or more, about 400 mg/ml or more, about 500 mg/ml or more, about 600 mg/ml or more, about 700 mg/ml or more, about 800 mg/ml or more, about 900 mg/ml or more, or about 1000 mg/ml or more.

In some embodiments, the biomarker present in a complex biological sample is at about 1 mg/ml or less, about 2 mg/ml or less, about 3 mg/ml or less, about 4 mg/ml or less, about 5 mg/ml or less, about 6 mg/ml or less, about 7 mg/ml or less, about 8 mg/ml or less, about 9 mg/ml or less, about 10 mg/ml or less, about 15 mg/ml or less, about 20 mg/ml or less, about 25 mg/ml or less, about 30 mg/ml or less, about 35 mg/ml or less, about 40 mg/ml or less, about 45 mg/ml or less, about 50 mg/ml or less, about 60 mg/ml or less, about 70 mg/ml or less, about 80 mg/ml or less, about 90 mg/ml or less, about 100 mg/ml or less, about 200 mg/ml or less, about 300 mg/ml or less, about 400 mg/ml or less, about 500 mg/ml or less, about 600 mg/ml or less, about 700 mg/ml or less, about 800 mg/ml or less, about 900 mg/ml or less, or about 1000 mg/ml or less.

### Proteomic Samplings

Proteins are essential cellular machinery, performing and enabling tasks within biological systems. The variety of proteins is extensive, and the role they occupy in biology is deep and complex; life depends on proteins. Each step of cellular generation, from replication of genetic material to cell senescence and death, relies on the correct function of several distinct proteins. The precision of cellular machinery can be disrupted, however, resulting in disease. Because much of the machinery essential to cell health and survival remains unknown, studying proteins is of great interest and importance. The field of proteomics is the large-scale study of proteins and the proteome and encompasses many techniques, such as, but not limited to, immunoassays and two-dimensional differential gel electrophoresis (2-D DIGE), and mass spectrometry. In some embodiments, mass spectrometry-based proteomics comprises top-down analysis. In some embodiments, mass spectrometry-based proteomics comprises bottom-up analysis. Top-down methods analyze whole proteins; bottom-up approaches investigate the peptides from digested proteins. There are unique methods of analysis that each group has developed, but they share in common their mode of analysis. In mass spectrometric analysis, the mass-to-charge ratios (m/z) of molecular species are determined. By collecting this data, compounds in the sample can be identified by comparing against standard databases of compounds and molecules with known masses. From whole protein analysis in top-down proteomics, to peptide analysis in bottom-up proteomics, each particle measured has an m/z signature detectable by the mass spectrometer. By pairing mass analyzers and detectors, adding equipment in different configurations, and coupling separations and mass spectrometers together, there are virtually limitless possibilities, functionalities, and speeds of data acquisition for mass spectrometry-based proteomic analysis. Mass spectrometry-based proteomics has advanced rapidly since the advent of “soft” ionization techniques, such as electrospray ionization (ESI) and matrix-assisted laser desorption ionization (MALDI). Methods of detection previously used for organic chemicals and other sample identifications were adapted for proteomics. Several combinations of ionization sources and mass analyzers are available commercially, each with merits for specific applications. Time-of-flight (TOF) instruments are often coupled with MALDI instruments. ESI-TOF instruments can provide high-speed, continuous measurements without compromising resolution. High-resolution Fourier Transform Ion Cyclotron Resonance (FT-ICR) mass spectrometers are costly, but provide unsurpassed data collection power using both electrospray and MALDI ion sources. In some cases, The Orbitrap mass analyzer provides a high mass resolution instrument at a lower cost than Fourier Transform Ion Cyclotron Resonance (FT-ICR). With mass spectrometric analysis, such as the Orbitrap, combined with the selectivity of ion traps and quadrupoles with analysis of complex samples with high mass accuracy and specificity, even in small quantities, can be completed with increasing ease and confidence.

A mass spectrometer is an instrument that measures the masses of individual molecules that have been converted into ions, i.e., molecules that have been electrically charged. A mass spectrometer measures the mass-to-charge (“m/z”) ratio of the ions formed from the molecules. The sample, which can be a solid, liquid, or vapor, enters a vacuum chamber through an inlet. Depending on the type of inlet and ionization techniques used, the sample can already exist as ions in solution, or it can be ionized in conjunction with its volatilization or by other methods in the ion source. The gas phase ions are sorted in the mass analyzer according to their m/z ratios and then collected by a detector. In the detector, the ion flux is converted to a proportional electrical current. A data system records the magnitude of these electrical signals as a function of m/z and converts this information into a mass spectrum. Further information on mass spectrometers and their use is described in U.S. Pat. Nos. 6,504,150 and 6,960,760, each of which is incorporated herein by its entirety.

The complex sample or complex biological sample as described herein can contain at least one type of a biomolecule within the complex sample. In some embodiments, the at least one type of a biomolecule comprises a biomarker of interest. In some embodiment, the biomarker of interest is not present in the sample. In some embodiments, the biomarker of interest is highly prevalent and has saturated the dynamic range of the test. In some embodiments, the biomarker of interest is present at an intermediate concentration.

In some embodiments, the fraction of a sample comprising a particular biomolecule at which binding occurs is mapped to the concentration of the binding biomarker in the sample. Typically, a correlation is made between the number of biomarkers of a given biomolecule type. By determining the number of bound biomolecules, a determination and/or estimate of the number and/or quantity of the specific biomarker (e.g., protein) present in the original sample can be made. However, this is not strictly necessary for a chemistry signature of the sample to be useful. Rather, the process can be repeatable and dispersive. That is, a range of species concentrations in the sample can produce a range of fractions of bound biomolecules, rather than collapsing onto a single fraction of bound biomolecules.

The ability of the biomarkers of a particular biomolecule type to saturate is an important aspect of the present disclosure, as it limits the level of influence that any one species, particularly a high abundance species, can have in the determined signature. The mass spectroscopy signal can be “clipped”, greatly improving the signal to noise ratio problem described earlier. By limiting the influence (i.e., clipping its signal) of one or more species that would otherwise be abundant in the sample, the mass spectrometer can more effectively detect variations of low abundance species as their signals are not overshadowed by high abundance species. Examples of high abundance proteins include, but are not limited to albumin, immunoglobulin, transferrin, factor H, C9 complement, C8 complement, C5 complement, C6 complement, C7 complement, fibrinogen; examples of low abundance protein include but are not limited to cytokines, signaling molecules, intracellular proteins, interleukin-4, TNF alpha, interferon gamma, interleukin-1 Beta, interleukin-12, interleukin-5, interleukin-10, and interleukin-6 (N. Leigh Anderson and Norman G. Anderson, The Human Plasma Proteome: History, Character, and Diagnostic Prospects, MOLECULAR & CELLULAR PROTEOMICS 1:845-867, 2002.). It should be understood that this is not an exhaustive list of high abundance proteins and low abundance proteins. One of skill in the art can recognize additional high abundance proteins and additional low abundance proteins that are in the biological sample. This is especially the case for low abundance proteins as there are a vast variety of low abundance proteins. The proteins (high abundance and low abundance) can be selected out of the sample and the signal of the high abundance proteins and low abundance proteins can be clipped or enhanced, respectively.

The proteomic sampling methods provided herein contemplate uses of various mass spectrometry methods to analyze proteins. Some methods are discovery-based, where samples are analyzed to determine what proteins are present in the sample. Often a high-resolution mass spectrometer is used for this purpose, as the false-discovery rate of protein identifications from peptides rely on highly accurate mass-to-charge measurements. Some methods are targeted, focusing on single proteins of interest and quantifying them in different samples or sample fractions. Highly selective methods using ion traps and quadrupoles are ideal for targeted analysis. Proteins and peptides can be fragmented in the mass spectrometer for tandem mass spectrometry in a variety of ways, and those fragments are analyzed for de novo peptide sequencing or peptide mass fingerprinting. The vast majority of peptide identifications are accomplished with fragmentation followed by protein database searches of the resulting fragments. Electron capture dissociation (ECD), electron transfer dissociation (ETD), higher energy collisional dissociation (HCD), collision-induced dissociation (CID), and a host of other fragmentation methods are available, each with recommended applications. Furthermore, mass spectrometry methods are often customized within software.

The mass spectrometer is a critical aspect in proteomic experiments; however, the results obtained from the mass spectrometer are limited by the sample. Regardless of the analysis approach used, a high quality sample is critical for a successful experiment. Proteomic analyses depend on the sample containing proteins to analyze. Sample preparation approaches that are time-consuming, or worse, incur massive sample losses, are intolerable. Techniques to produce proteomic sampling of e.g., plasma proteome without a prior depletion and independent of e.g., plasma protein concentration and to generate a large training and test data set of disease labeled biomarker protein levels across many patient sample are described in International Patent Application PCT/US2017/067013, which is herein incorporated by reference in its entirety. Also contemplated herein are various other proteomic sampling preparation methods, such as affinity purification, size exclusion, hydrophobic exclusion, charge exclusion. Also contemplated herein is the creation of a relational database in which the surface-activity relationships between biomarkers and capture particles such as coronas are analyzed in order to rationally design capture particles. In order to obtain protein from a complex biological sample, the sample must first be harvested from the organism, culture, or patient. Samples can be obtained by several methods. Traditional dissection from animal species, biopsies, blood draws, and additional methods can deliver adequate protein for analysis. The proteins in the sample must be made readily accessible via lysis and extraction from the cells in the sample.

Numerous methods for lysis and extraction are well known in the art, e.g., lysis buffers, mechanical disruption strategies, a chemical lysis and extraction agent, along with some mechanical stimulus that physically breaks apart the cell, allowing the chemical agent to solubilize the available protein. Non-limiting exemplary common detergents for mass spectrometry and cell lysis include Triton X-100, NP-40, Tween 20, Tween 80, octyl glucoside, octyl thioglucoside, Big CHAP, deoxycholate, sodium dodecyl sulfate, CHAPS, and CHAPSO. Lysis buffers can differ in critical micelle concentration (CMC). The CMC is the concentration at which the detergent forms micelles spontaneously, which can affect their efficacy and removal in different environments. Above this point, the detergent forms micelles, and detergent added can move directly into micelles. Higher CMC values are associated with weaker hydrophobic binding to monomers. Thus, higher CMC detergents tend to be more easily removed by buffer exchange and dialysis. Solutions with lower CMC values form micelles more easily, and generally require less detergent to effectively solubilize protein. Another factor that can affect a lysis buffer is the micelle molecular weight (MMW). Lower-weight micelles are more easily removed than higher-weight micelles. Making use of CMC and MMW to determine the best course of the experiment is well known in the art. Choosing a lysis buffer depends greatly on these detergent factors. Most of the detergents listed are incompatible with downstream mass spectrometry analysis, and must be removed. Lysis, extraction, and denaturation of protein can occur in the same step with certain procedures, such as with sodium dodecyl sulfate SDS while boiling and agitating the sample. See Bodzon-Kulakowska, A. et al. Methods for samples preparation in proteomic research. J. Chromatogr. 2007, 849, 1-31; Visser, N. F. C. et al. Sample preparation for peptides and proteins in biological matrices prior to liquid chromatography and capillary zone electrophoresis. Anal. Bioanal. Chem. 2005, 382, 535-558; Hilbrig, F. and Freitag, R. Protein purification by affinity precipitation. J. Chromatogr. 2003, 790, 79-90; Zhou, J.-Y. et al. Simple sodium dodecyl sulfate-assisted sample preparation method for LC-MS-based proteomics applications. Anal. Chem. 2012, 84, 2862-2867.

Once protein is extracted, removal of contaminants and detergents is necessary. Some detergents can interfere with enzymatic digestion. Some detergents can interfere with reverse-phase separations and mass spectrometry. Removal of unwanted cellular material, such as lipids and genomic DNA, prevents signal suppression, chromatographic interference, and presents a much cleaner, clearer spectrum from which to obtain protein identification data. Various methods of contaminant removal are well known in the art. Non-limiting exemplary methods of contaminant removal include precipitation, salting out, ultrafiltration, polyethyleneimine (PEI), isoelectric point (PI), thermal, and nonionic polymer polyethylene glycol (PEG). See Englard, S. and Seifter, S. Precipitation techniques. Methods Enzymol. 1990, 182, 285-300; Burgess, R. R. Protein precipitation techniques. Methods Enzymol. 2009, 463, 331-342; Jiang, L. et al. Comparison of protein precipitation methods for sample preparation prior to proteomic analysis. J. Chromatogr. 2004, 1023, 317-320; Wisniewski, J. R. et al. Comparison of ultrafiltration units for proteomic and N-glycoproteomic analysis by the filter-aided sample preparation method. Anal. Biochem. 2011, 410, 307-309. Int. J. Mol. Sci. 2015, 16 3557; Gupta, M. N. et al. Affinity precipitation of proteins. J. Mol. Recognit. 1996, 9, 356-359; Holler, C. et al. Polyethyleneimine precipitation versus anion exchange chromatography in fractionating recombinant 3-glucuronidase from transgenic tobacco extract. J. Chromatogr. 2007, 1142, 98-105; Hegg, P. O. Precipitation of egg white proteins below their isoelectric points by sodium dodecyl sulphate and temperature. Biochim. Biophys. Acta 1979, 579, 73-87; Jaffe, W. G. A simple method for the approximate estimation of the isoelectric point of soluble proteins. J. Biol. Chem. 1943, 148, 185-186; Fan, J. et al. Thermal precipitation fluorescence assay for protein stability screening. J. Struct. Biol. 2011, 175, 465-468; Hill, A. R. and Irvine, D. M. Effects of pH on the thermal precipitation of proteins in acid and sweet cheese wheys. Can. Inst. Food Sci. Technol. J. 1988, 21, 386-389; Ingham, K. C. Precipitation of proteins with polyethylene glycol. Methods Enzymol. 1990, 182, 301-306; Ingham, K. C. Protein precipitation with polyethylene glycol. Methods Enzymol. 1984, 104, 351-356; Sim, S.-L. et al. Protein precipitation by polyethylene glycol: A generalized model based on hydrodynamic radius. J. Biotechnol. 2012, 157, 315-319; Crowell, A. M. J. et al. Maximizing recovery of water-soluble proteins through acetone precipitation. Anal. Chim. Acta 2013, 796, 48-54; Barritault, D. et al. The use of acetone precipitation in the isolation of ribosomal proteins. Eur. J. Biochem. 1976, 63, 131-135; Puchades, M. et al. Analysis of intact proteins from cerebrospinal fluid by matrix-assisted laser desorption/ionization mass spectrometry after two-dimensional liquid-phase electrophoresis. Rapid Commun. Mass Spectrom. 1999, 13, 2450-2455; Thongboonkerd, V. et al. Proteomic analysis of normal human urinary proteins isolated by acetone precipitation or ultracentrifugation. Kidney Int. 2002, 62, 1461-1469; Srivastava, O. P. et al. Purification of gamma-crystallin from human lenses by acetone precipitation method. Curr. Eye Res. 1998, 17, 1074-1081; Von Hagen, J. Proteomics Sample Preparation; John Wiley & Sons Inc.: Hoboken, N.J., USA, 2011; Wu, X. et al. Universal sample preparation method integrating trichloroacetic acid/acetone precipitation with phenol extraction for crop proteomic analysis. Nat. Protoc. 2014, 9, 362-374; Chevallet, M. et al. Toward a better analysis of secreted proteins: The example of the myeloid cells secretome. Proteomics 2007, 7, 1757-1770; Robinson, P. J. et al. Activation of protein kinase C in vitro and in intact cells or synaptosomes determined by acetic acid extraction of MARCKS. Anal. Biochem. 1993, 210, 172-178. Int. J. Mol. Sci. 2015, 16 3558; Isaacson, T. et al. Sample extraction techniques for enhanced proteomic analysis of plant tissues. Nat. Protoc. 2006, 1, 769-774. Duan, X. et al. A straightforward and highly efficient precipitation/on-pellet digestion procedure coupled with a long gradient nano-LC separation and orbitrap mass spectrometry for label-free expression profiling of the swine heart mitochondrial proteome. J. Proteome Res. 2009, 8, 2838-2850.

Non-limiting exemplary approaches for bottom-up proteomic analysis include in-solution and in-gel digestion. In-solution digestion involves denaturing, reducing, alkylating, and digesting the protein sample in the liquid phase, as opposed to in a gel or on a filter. In-solution digestion is well known in the art. See De Godoy, L. M. F. et al. Comprehensive mass-spectrometry-based proteome quantification of haploid versus diploid yeast. Nature 2008, 455, 1251-1254; Li, N. et al. Lipid raft proteomics: Analysis of in-solution digest of sodium dodecyl sulfate-solubilized lipid raft proteins by liquid chromatography-matrix-assisted laser desorption/ionization tandem mass spectrometry. Proteomics 2004, 4, 3156-3166; De Souza, G. A. et al. Identification of 491 proteins in the tear fluid proteome reveals a large number of proteases and protease inhibitors. Genome Biol. 2006, 7, R72; Go, E. P. et al. In-solution digestion of glycoproteins for glycopeptide-based mass analysis. Methods Mol. Biol. (Clifton N.J.) 2013, 951, 103-111. In-solution digestion can be performed using single-tube approaches, eliminating much of the sample loss that occurs during solution transfer between different vessels. Generally in-solution digests are fractionated after digestion, but fractionation can be performed previous to digestion using different forms of chromatography, including, but not limited to, strong and weak ion exchange, reverse-phase, and size exclusion chromatography.

Gel-based mass spectrometry analysis is well known in the art and widely used as a first method of separation prior to LC-MS/MS analysis. See Lasonder, E. et al. Analysis of the Plasmodium falciparum proteome by high-accuracy mass spectrometry. Nature 2002, 419, 537-542; Pomastowski, P. and Buszewski, B. Two-dimensional gel electrophoresis in the light of new developments. TrAC Trends Anal. Chem. 2014, 53, 167-177; Shevchenko, A. et al. Mass spectrometric sequencing of proteins from silver-stained polyacrylamide gels. Anal. Chem. 1996, 68, 850-858. Different methods of gel electrophoresis are well known in the art. Before the digestion, separation of the protein is performed using a gel. In one of the most common proteomic sample preparation strategies, a denaturing gel (sodium dodecyl sulfate in a polyacrylamide gel, SDS-PAGE) is used for bottom-up proteomics, as the protein can be cleaved into peptides in later steps. The gel is used to separate whole protein in one or two dimensions. After destaining, the proteins are excised from the gel and subjected to enzymatic proteolysis. Peptides can then be analyzed via mass spectrometry. The stained gel pieces are then excised from the gel, destained, and the protein within the gel piece is subjected to digestion. Strategies for in-gel digestions are well known in the art.

Various mass spectrometry and fractionation combinations for delving deeply into the proteome of organisms and model systems are well known in the art. For example, for the yeast proteome, the multi-dimensional protein identification technology is used for the yeast proteome. Accurate mass tags (AMT) were developed in order to decrease the need for tandem mass spectrometry while providing more sensitive measurements and greater dynamic range. High-performance liquid chromatography (HPLC) is a very common separation technique with a wide variety of stationary phases. With the advent of ultra-performance liquid chromatography (UPLC), chromatographic separations have increased both in resolving power and speed of separation. HPLC and UPLC function on the same principles. UPLC columns generally offer smaller particle sizes, resulting in decreased analyte path length and higher column pressures (10,000 pounds per square inch (PSI) or greater in maximum). Liquid Chromatography is often coupled with electrospray ionization and tandem mass spectrometers for both top-down and bottom-up proteomic analysis. LC has been used for a staggering number of analyses; LC-MS is a commonly used for proteomic analysis. Liquid chromatography is robust, customizable based on the functionality of the stationary particles in the separation column. For bottom-up proteomic analysis, the most common HPLC/UPLC stationary phase is the C18 reverse-phase column. The reverse-phase column uses the hydrophobicity of peptides for separation, utilizing a gradient from low to high organic-phase solvent. Acidified methanol and acetonitrile are commonly used as organic-phase, also known as “B” or “strong” solvents because of their miscibility with aqueous solutions. Acidified water is most often the “weak” solvent, also known as “A”. Both buffers are acidified with the same acid, generally with formic acid or trifluoroacetic acid (TFA) at 0.1% or 0.01%, respectively. Formic acid is preferred over TFA, as TFA tends to form adducts and suppress signal. While reverse-phase columns are commonly used, many stationary phases are in use for proteomic work in both one- and two-dimensional separations, online and off-line. A separation strategy known as electrostatic repulsion hydrophilic interaction chromatography (ERLIC) has gained popularity for phosphoproteomic work, using adjustments in pH and volatile salts for gradient separations. As the name suggests, ERLIC uses the charge and hydrophilicity of peptides as a basis for separation. Typically ERLIC begins with a low-organic, high-pH gradient, moving to high organic and low-pH as the separation moves on. In this way, ERLIC elutes peptides in order of increasing hydrophobicity and acidity. ERLIC has proven effective at separating and identifying modified and unmodified proteins. Smaller-diameter columns with lower loading capacities and smaller stationary phase particles offer an advantage in microproteomics. By increasing the local concentration of peptide and decreasing eddy diffusion, sample loading amounts can be minimized and still provide adequate peptide signal; the chromatographic resolution necessary for complex sample separation is not compromised. See Washburn, M. P. et al. Large-scale analysis of the yeast proteome by multidimensional protein identification technology. Nat. Biotechnol. 2001, 19, 242-247; Lipton, M. S. et al. Global analysis of the Deinococcus radiodurans proteome by using accurate mass tags. Proc. Natl. Acad. Sci. USA 2002, 99, 11049-11054; Xie, F. et al. Liquid chromatography-mass spectrometry-based quantitative proteomics. J. Biol. Chem. 2011, 286, 25443-25449; Peng, J. et al. Evaluation of multidimensional chromatography coupled with tandem mass spectrometry (LC/LC-MS/MS) for large-scale protein analysis: The yeast proteome. J. Proteome Res. 2003, 2, 43-50; Jungblut, P. R. Protein and Peptide Mass Spectrometry in Drug Discovery; Gross, M. L., Chen, G., Pramanik, B., Eds.; ChemMedChem: Weinheim, Germany, 2012; Volume 7, pp. 2241-2242.

### Application of Complex Biomolecule Samplings

The present disclosure relates to machine learning algorithms for a complex biomolecule sampling (e.g., proteome sampling) from a subject. The algorithms provided herein can aid in selection of previously unknown biomarkers and provide a report comprising a score or probability relating to, for example, disease risk, disease likelihood, presence or absence of disease, treatment response, and/or classification of disease status.

Methods of diagnosing or prognosing a disease or disorder using the biomarkers identified by the present methods are also contemplated. The methods comprise obtaining a sample from a subject; contacting the sample with e.g., a plurality of nanoparticles to produce a complex biomolecule sampling data, and comparing the complex biomolecule sampling data to the known cohort of biomolecule data identified in the disease or disorder; and diagnosing or prognosing the disease or disorder based on the presence of one of more of the identified biomarkers.

In some embodiments, methods of identifying patterns of biomarkers or specific biomarkers associated with a disease or disorder are contemplated. Suitable methods, include, for example, preforming the methods described above (e.g. obtaining a samples from at least two subjects diagnosed with the disease or disorder and at least two control subjects; contacting each sample with the sensor array to produce a biomolecule fingerprint, and comparing the biomolecule fingerprint of the subjects with the disease or disorder to the biomolecule fingerprint of the control subjects to determine at least one pattern and/or biomarker associated with the disease or disorder. Suitable, the method can comprise at least 2 disease subjects and at least two control subjects, alternatively at least 5 disease subjects and at least 5 control subjects, alternatively at least 10 disease subjects and at least 10 control subjects, alternatively at least 15 disease subjects and at least 15 control subjects, alternatively at least 20 disease subjects and at least 20 control subjects, and includes any variations in between (e.g. disease subjects from at least 2-100, and control subjects from at least 2-100).

In some embodiments, the arrays and methods allow for the determination of a pattern of biomarkers associated with the disease state or disease or disorder or, in some embodiments, specific biomarkers that are associated with the disease or disorder. Not only will biomarkers that can be associated with a disease state be able to be identified, for example, biomarkers listed herein, but new biomarkers or patterns of biomarkers that can be associated with a disease state or a disease or disorder can be determined. As discussed above, some biomarkers or patterns of biomarkers for a specific disease or disorder can be a change in a biomolecule associated with the sensor array of the present disclosure and differ from what is usually referred to as biomarkers in the art, e.g., and increase expression of a specific biomolecule associated with a disease. As discussed above, it can be the interaction of a biomolecule, e.g. biomolecule X, with other biomolecules, e.g. biomolecule Y and Z, that results in the ability to associate with a specific disease state and cannot correlate with any change in the absolute concentration of biomarker X in the sample over time or disease state. Thus, a molecule that would not in the conventional sense be considered a biomarker since it does not change in absolute concentration in a sample from the pre-disease to disease state, can in view of the present disclosure be considered a biomolecule as its relative changes that are measured by the array of the present disclosure are associated with a disease state. In other words, it can be an increase or decrease in the interaction of biomolecule X (due to the interactions of X with the sensor elements and other biomolecules in the sample) with the array that provides a signal that a biomarker is associated with a disease state.

Any of the methods, kits, and systems described herein can utilize a diagnostic assay for predicting a disease status of a subject or likelihood of a subject's response to a therapeutic. The diagnostic assay can use the presence of one or more biomarkers identified using the methods described herein to calculate a quantitative score that can be used to predict disease status or likelihood of response to a therapeutic in a subject. The diagnostic assay can use the presence of one or more biomarkers and one or more characteristics, such as, e.g., age, weight, gender, medical history, risk factors, family history to calculate a quantitative score that can be used to predict disease status or likelihood of response to a therapeutic in a subject.

In some applications, an increase in a score in the diagnostic assay indicates an increased likelihood of one or more of: a poor clinical outcome, good clinical outcome, high risk of disease, low risk of disease, complete response, partial response, stable disease, non-response, and recommended treatments for disease management. In some embodiments, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

In some applications, a decrease in a score in the diagnostic assay indicates an increased likelihood of one or more of: a poor clinical outcome, good clinical outcome, high risk of disease, low risk of disease, complete response, partial response, stable disease, non-response, and recommended treatments for disease management. In some embodiments, a decrease in the quantitative score indicates an increased likelihood of one or more of: a poor clinical outcome, good clinical outcome, high risk of disease, low risk of disease, complete response, partial response, stable disease, non-response, and recommended treatments for disease management.

Also provided herein are methods for generalized-treatment recommendations for a subject based on their complex biomolecule samplings and methods for subject-speific treatment recommendation. Methods for treatments can comprise following steps: detecting a presence or absence of one or more biomarkers specific for a disease state, such as cancer; recommending to the subject at least one generalized or subject-speific treatment to ameliorate disease symptoms; and monitoring of the disease progression, treatment responses, and recurrence of the disease by detecting the one or more biomarkers.

Methods provided herein can be applied to, for example, tumor (cancer) analysis. Cancer is one of the leading causes of death in the United States, and the growth and developmental mechanisms of tumors are poorly understood. Tumors have substantial cellular heterogeneity, making tumor biology a critical area of study. Methods disclosed herein provide valuable tools to explore the complex proteomic differences within e.g., a single tumor. Tumor biology and biomarker discovery are major driving forces for proteomic analysis, based on the number of publications on proteomic cancer analysis in recent years. Proteomics is useful for tumor biology due to the breadth of protein information obtainable. Often, diagnosis and evaluation of tumors are done with histology and immunohistochemical analysis. Biopsies are sliced, stained, and analyzed with microscopy. While accurate, precise, and mature, the throughput of this technique is relatively low. Immunostaining and histochemical staining are commonly used for cancer diagnosis, but they are limited techniques. Only one or two proteins can be visualized with traditional microscopy techniques. Tumors can also be biopsied using needle core or aspiration biopsies. The typical diameter of a needle core biopsy is about 1 mm across and about the length of a grain of rice, limiting sample amount and thus the breadth of analyses that can be possible. The methods provided herein can bridge this gap. Diagnosis, proteome analysis, and network analysis can be consolidated, and the methods provided herein can help ensure maximum data from minimal material. Thousands of protein groups can be identified in a single mass spectrometry run. In some embodiments, no prior protein depletion is required. That information is then uploaded into network analysis databases, providing an in-depth look at a tumor's molecular equilibria. The methods provided herein can identify targets for cancer therapy. Analysis of pathways in cancer can not only give insight into the workings of cancer; it can also give more immediate treatment options, possibly ruling out ineffective therapies or encouraging more productive, less deleterious chemotherapies.

Suitable cancer biomarkers include, but are not limited to, for example, AHSG (a2-HS-Glycoprotein), AKR7A2 (Aflatoxin B1 aldehyde reductase), AKT3 (PKB y), ASGR (ASGPR1), BDNF, BMP1 (BMP-1), BMPER, C9, CA6 (Carbonic anhydrase VI), CAPG (CapG), CDH1 (Cadherin-1), CHRDL1 (Chordin-Like 1), CKB-CKM-(CK-MB), CLIC1 (chloride intracellular channel 1), CMA1 (Chymase), CNTN1 (Contactin-1), COL18A1 (Endostatin), CRP, CTSL2 (Cathepsin V), DDC (dopa decarboxylase), EGFR (ERBB1), FGA-FGB-FGG (D-dimer), FN1 (Fibronectin FN1.4), GHR (Growth hormone receptor), GPI (glucose phosphate isomerase), HMGB1 (HMG-1), HNRNPAB (hnRNP A/B), HP (Haptoglobin, Mixed Type), HSP90AA1 (HSP 90a), HSPA1A (HSP 70), IGFBP2 (IGFBP-2), IGFBP4 (IGFBP-4), IL12B-IL23A (IL-23), ITIH4 (Inter-a-trypsin inhibitor heavy chain H4), KIT (SCF sR), KLK3-SERPINA3 (PSA-ACT), LlCAM (NCAM-L1), LRIG3, MMP12(MMP-12), MMP7 (MMP-7), NME2 (NDP kinase B), PA2G4 (ErbB3 binding protein Ebpl), PLA2G7 (LpPLA2/PAFAH), PLAUR (suPAR), PRKACA (PRKA C-a), PRKCB (PKCp-n), PROKI (EG-VEGF), PRSS2 (Trypsin-2), PTN (Pleiotrophin), SERPINA1 (a1-Antitrypsin), STC1 (Stanniocalcin-1), STX1A (Syntaxin 1A), TACSTD2 (GA733-1 protein), TFF3 (Trefoil factor 3), TGFBI (13IGH3), TPI1 (Triosephosphate isomerase), TPT1 (Fortilin), YWHAG (14-3-3 protein y), YWHAH (14-3-3 protein eta), prostate cancer biomarkers, for example, PSA, Pro-PSA, PHI, PCA3, TMPRSS3:ERG, PCMT, MTEN, breast cancer markers, for example, epidermal growth factor receptor 2 (HER2) oncogene, melanoma biomarker BRAF, lung cancer biomarker EML4-ALK, A2ML1, BAX, C10orf47, Clorfl62, CSDA, EIFC3, ETFB, GABARAPL2, GUKI, GZMH, HIST1H3B, HLA-A, HSP90AA1, NRGN, PRDXS, PTMA, RABACI, RABAGAP1L, RPL22, SAP 18, SEPW1, SOX1, EGFR, EGFRvIII, apolipoprotein A, apolipoprotein CIII, myoglobin, tenascin C, MSH6, claudin-3, claudin-4, caveolin-1, coagulation factor III, CD9, CD36, CD37, CD53, CD63, CD81, CD136, CD147, Hsp70, Hsp90, Rabl3, Desmocollin-1, EMP-2, CK7, CK20, GCDF15, CD82, Rab-5b, Annexin V, MFG-E8, HLA-DR, a miR200 microRNA, MDC, NME-2, KGF, PIGF, Flt-3L, HGF, MCP1, SAT-1, MIP-1-b, GCLM, OPG, TNF RII, VEGF-D, ITAC, MMP-10, GPI, PPP2R4, AKR1B1, AmylA, MIP-lb, P-Cadherin, EPO and the like. For example, biomarkers for breast cancer include, but are not limited to, ER/PR, HER-2/neu, and the like. Biomarkers for colorectal cancer include, but are not limited to, for example, EGFR, KRAS, UGT1A1, and the like. Biomarkers associated with leukemia/lymophoma include, but are not limited to, e.g., CD20 antigen, CD30, FIPL1-PDGFRalpha, PDGFR, Philladelphia Chromosome (BCR/ABL), PML/RAR alpha, TPMT, UGT1A1, and the like. Biomarker associated with lung cancer include but are not limited to, e.g., ALK, EGFR, KRAS and the like. Biomarkers are known in the art, and can be found in, for example, Bigbee W, Herberman R B. Tumor markers and immunodiagnosis. In: Bast R C Jr., Kufe D W, Pollock R E, et al., editors. Cancer Medicine. 6th ed. Hamilton, Ontario, Canada: BC Decker Inc., 2003; Andriole G, Crawford E, Grubb R, et al. Mortality results from a randomized prostate-cancer screening trial. New England Journal of Medicine 2009; 360(13):1310-1319; Schroder F H, Hugosson J, Roobol M J, et al. Screening and prostatecancer mortality in a randomized European study. New England Journal of Medicine 2009; 360(13):1320-1328; Buys S S, Partridge E, Black A, et al. Effect of screening on ovarian cancer mortality: the Prostate, Lung, Colorectal and Ovarian (PLCO) Cancer Screening Randomized Controlled Trial. JAMA 2011; 305(22):2295-2303; Cramer D W, Bast R C Jr, Berg C D, et al. Ovarian cancer biomarker performance in prostate, lung, colorectal, and ovarian cancer screening trial specimens. Cancer Prevention Research 2011; 4(3):365-374; Sparano J A, Gray R J, Makower D F, et al. Prospective validation of a 21-gene expression assay in breast cancer. New England Journal of Medicine 2015; First published online Sep. 28, 2015. doi: 10.1056/NEJMoa1510764, incorporated by reference in their entireties.

Biomarkers may also be associated with the cardiovascular disease which are known in the art and include, but are not limited to, lipid profile, glucose, and hormone level and physiological biomarkers based on measurement of levels of important biomolecules such as serum ferritin, triglyceride to HDLp (high density lipoproteins) ratio, lipophorin-cholesterol ratio, lipid-lipophorin ratio, LDL cholesterol level, HDLp and apolipoprotein levels, lipophorins and LTPs ratio, sphingolipids, Omega-3 Index, and ST2 level, among others. Suitable biomarkers for cardiovascular disease can be found in the art, for example, but not limited to, in van Holten et al. “Circulating Biomarkers for Predicting Cardiovascular Disease Risk; a Systemic Review and Comprehensive Overview of Meta-Analyses” PLoS One, 2013 8(4): e62080, incorporated by reference in its entirety. Biomarkers may also be associated with a neurological disease. Suitable biomarkers are known in the art and include, but are not limited to, e.g., A131-42, t-tau and p-tau 181, a-synuclein, among others. See, e.g., Chintamaneni and Bhaskar” Biomarkers in Alzheimer's Disease: A Review” ISRN Pharmacol. 2012. 2012: 984786. Published online 2012 Jun. 28, incorporated by reference in its entirety. Biomarkers for inflammatory diseases are known in the art and include, but are not limited to, e.g., cytokines/chemokines, immune-related effectors, acute-phase proteins [C-reactive protein (CRP) and serum amyloid A (SAA)], reactive oxygen species (ROS) and reactive nitrogen species (RNS), prostaglandins and cyclooxygenase (COX)-related factors, and mediators such as transcription factors and growth factors, which can include, for example, C-reactive protein (CRP), 5100, LIF, CXCL1, CXCL2, CXCL4, CXCL5, CXCL8, CXCL9, CXCL10, CCL2, CCL23, IL-113, IL-IRa, TNF, IL-6, IL-10, IL-17A, IL-17F, IL-21, IL-22, IFNy, CXCR1, CXCR4, CXCR5, GM-CSF, GM-CSFR, G-CSF, G-CSFR, EGF, VEGFA, LEP, SAA1, VCAM1, CRP, MMP1, MMP3, TNFRSF1A, RETN, CHI3L1, antinuclear antibodies (ANA), rheumatoid factor (RF), antibodies against cyclic citrullinated peptide (anti-CCP)] and for chronic IBD (fecal calprotectin), among others. Suitable biomarkers for inflammatory bowel disease, for example, include CRP, ESR, pANCA, ASCA, and fecal calprotectin. See, e.g., Yi Fengming and Wu Jianbing, “Biomarkers of Inflammatory Bowel Disease,” Disease Markers, vol. 2014, Article ID 710915, 11 pages, 2014. doi:10.1155/2014/710915, incorporated by reference in its entirety.

TABLE 1
Exemplary genes encoding disease labeled proteins

| | | Uniprot- | Gene | Lung | Pancreatic | Brain | Multiple | | |
|---|---|---|---|---|---|---|---|---|---|
| Uniprotkb-ID | Score | ID | name | Carcinoma | Carcinoma | Neoplasm | Myeloma | Carcinoma | Cancer |
| CBPN_HUMAN | 13.38 | P15169 | CPN1 | | 0.05 | | | 0.05 | 0.09 |
| FCN3_HUMAN | 12.66 | O75636 | FCN3 | 0 | | 0.03 | | 0.18 | 0.18 |
| CO7_HUMAN | 12.48 | P10643 | C7 | 0.54 | 0 | 0 | | 0.68 | 0.68 |
| SAA4_HUMAN | 12.38 | P35542 | SAA4 | | | 0.02 | | 0.05 | 0.05 |
| FIBB_HUMAN | 10.89 | P02675 | FGB | 0.78 | | 0.81 | 0.77 | 0.82 | 1 |
| APOA2_HUMAN | 10.25 | P02652 | APOA2 | 0.05 | 0.15 | | 0.02 | 0.69 | 0.69 |
| IGHA1_HUMAN | 10.18 | P01876 | IGHA1 | | | | | 0.01 | 0.02 |
| IGHG1_HUMAN | 9.77 | P01857 | IGHG1 | 0.03 | 0.06 | 0.02 | | 0.09 | 0.09 |
| APOA1_HUMAN | 9.75 | P02647 | APOA1 | 0.06 | 0.04 | 0.04 | 0.02 | 0.8 | 0.8 |
| FIBA_HUMAN | 9.61 | P02671 | FGA | 0.79 | 0.81 | 0.8 | | 0.83 | 1 |
| HBA_HUMAN | 9.56 | P69905 | HBA1 | 0.75 | | | | 0.98 | 1 |
| HBA_HUMAN | 9.56 | P69905 | HBA2 | | | | | | 0.24 |
| CO8G_HUMAN | 9.35 | P07360 | C8G | | | | | 0.61 | 0.61 |
| FIBG_HUMAN | 9.23 | P02679 | FGG | 0.78 | | 0.8 | | 0.83 | 1 |
| HABP2_HUMAN | 8.94 | Q14520 | HABP2 | 0.16 | | | | 0.86 | 0.86 |
| APOB_HUMAN | 8.79 | P04114 | APOB | 0.79 | 0.57 | 0.73 | 0.42 | 0.83 | 0.96 |
| IGHG3_HUMAN | 8.63 | P01860 | IGHG3 | | 0.01 | | 0.04 | 0.02 | 0.05 |
| IGHG2_HUMAN | 8.59 | P01859 | IGHG2 | | | | | 0.02 | 0.02 |
| VTDB_HUMAN | 8.48 | P02774 | GC | 0.72 | 0.04 | 0.15 | 0.03 | 0.83 | 0.83 |
| FHR5_HUMAN | 8.44 | Q9BXR6 | CFHR5 | | | 0.01 | | 0.02 | 0.02 |
| CO4B_HUMAN | 8.34 | P0C0L5 | C4B | | | | | 0.05 | 0.06 |
| CO4B_HUMAN | 8.34 | P0C0L5 | C4B_2 | | | | | | |
| GELS_HUMAN | 8.17 | P06396 | GSN | 0.13 | 0.07 | 0.64 | 0.01 | 0.82 | 0.84 |
| IGLL5_HUMAN | 8.08 | B9A064 | IGLL5 | | | | | | 0.03 |
| COF1_HUMAN | 7.79 | P23528 | CFL1 | 0.08 | 0.04 | 0.16 | | 0.73 | 0.74 |
| COL11_HUMAN | 7.71 | Q9BWP8 | COLEC11 | 0.68 | | | | 0.71 | 0.71 |
| A2MG_HUMAN | 7.7 | P01023 | A2M | 0.68 | 0.04 | 0.55 | 0.04 | 0.81 | 0.82 |
| CO6_HUMAN | 7.65 | P13671 | C6 | 0.54 | 0 | | | 0.68 | 0.68 |
| HBB_HUMAN | 7.37 | P68871 | HBB | 0.89 | | 0.01 | 0.02 | 1 | 1 |
| CO8A_HUMAN | 7.17 | P07357 | C8A | 0.54 | | | | 0.69 | 0.69 |
| C1S_HUMAN | 6.88 | P09871 | C1S | 0.22 | | 0 | | 0.3 | 0.3 |
| PROP_HUMAN | 6.66 | P27918 | CFP | 0.09 | 0.04 | 0.08 | 0.03 | 0.64 | 0.64 |
| RET4_HUMAN | 6.63 | P02753 | RBP4 | 0.04 | 0.06 | 0.22 | 0.03 | 0.6 | 0.62 |
| VTNC_HUMAN | 6.62 | P04004 | VTN | 0.53 | 0.07 | 0.15 | 0.03 | 0.73 | 0.74 |
| MASP1_HUMAN | 6.48 | P48740 | MASP1 | 0.69 | | 0.02 | | 0.72 | 0.73 |
| PLMN_HUMAN | 6.42 | P00747 | PLG | 0.46 | 0.63 | 0.14 | | 1 | 1 |
| GRP78_HUMAN | 6.36 | P11021 | HSPA5 | 0.14 | 0.18 | 0.24 | 0.15 | 0.76 | 0.76 |
| C4BPA_HUMAN | 6.28 | P04003 | C4BPA | 0.04 | 0.03 | | | 0.21 | 0.22 |
| CO9_HUMAN | 6.21 | P02748 | C9 | 0.54 | | | | 0.68 | 0.68 |
| C1R_HUMAN | 5.93 | P00736 | C1R | 0.02 | 0.02 | 0 | 0.01 | 0.26 | 0.26 |
| APOD_HUMAN | 5.84 | P05090 | APOD | | 0.01 | 0.07 | | 0.19 | 0.19 |
| KV118_HUMAN | 5.77 | | | | | | | | |
| ZPI_HUMAN | 5.75 | Q9UK55 | SERPINA10 | 0.06 | | | | 0.14 | 0.16 |
| CPN2_HUMAN | 5.73 | P22792 | CPN2 | | | | | 0.07 | 0.07 |
| TRFE_HUMAN | 5.7 | P02787 | TF | 0.52 | 0.07 | 0.11 | 0.06 | 0.74 | 0.74 |
| VWF_HUMAN | 5.68 | P04275 | VWF | 0.8 | 0.03 | 0.86 | 0.78 | 0.86 | 1 |
| ALBU_HUMAN | 5.58 | P02768 | ALB | 0.53 | 0.04 | 0.23 | 0.06 | 0.82 | 0.84 |
| CO5_HUMAN | 5.53 | P01031 | C5 | 0.54 | 0.05 | 0.73 | | 0.78 | 0.97 |
| FINC_HUMAN | 5.49 | P02751 | FN1 | 0.83 | 0.84 | 0.85 | 0.08 | 1 | 1 |
| FA5_HUMAN | 5.42 | P12259 | F5 | 0.69 | 0 | 0.64 | 0.01 | 0.78 | 0.79 |
| CALD1_HUMAN | 5.35 | Q05682 | CALD1 | 0.04 | | 0.05 | | 0.5 | 0.51 |
| APOH_HUMAN | 5.31 | P02749 | APOH | 0.01 | 0.01 | | 0.01 | 0.67 | 0.67 |
| FGL1_HUMAN | 5.26 | Q08830 | FGL1 | 0.01 | 0 | | | 0.05 | 0.05 |
| FETUA_HUMAN | 5.16 | P02765 | AHSG | 0.05 | 0.04 | 0.08 | 0.04 | 0.17 | 0.17 |
| ITIH2_HUMAN | 5.14 | P19823 | ITIH2 | | 0 | | | 0.07 | 0.07 |
| COMP_HUMAN | 5.12 | P49747 | COMP | 0.03 | 0.02 | | | 0.6 | 0.6 |
| FA10_HUMAN | 5.11 | P00742 | F10 | 0.72 | 0.55 | 0.63 | 0.03 | 0.78 | 1 |
| H4_HUMAN | 5.04 | P62805 | HIST1H4A | | | | | | |
| H4_HUMAN | 5.04 | P62805 | HIST1H4B | | | | | | 0.02 |
| H4_HUMAN | 5.04 | P62805 | HIST1H4C | | | | | | 0 |
| H4_HUMAN | 5.04 | P62805 | HIST1H4D | 0.1 | 0.04 | 0.04 | | 0.12 | 0.13 |
| H4_HUMAN | 5.04 | P62805 | HIST1H4E | | | | | | |
| H4_HUMAN | 5.04 | P62805 | HIST1H4F | | | | | | |
| H4_HUMAN | 5.04 | P62805 | HIST1H4H | 0.4 | | | | 0.4 | 0.4 |
| H4_HUMAN | 5.04 | P62805 | HIST1H4I | 0.79 | | 0.56 | | 0.89 | 0.89 |
| H4_HUMAN | 5.04 | P62805 | HIST1H4J | | | | | | |
| H4_HUMAN | 5.04 | P62805 | HIST1H4K | | | | | | |
| H4_HUMAN | 5.04 | P62805 | HIST1H4L | | | | | | |
| H4_HUMAN | 5.04 | P62805 | HIST2H4A | | | | | | |
| H4_HUMAN | 5.04 | P62805 | HIST2H4B | | | | | | |
| H4_HUMAN | 5.04 | P62805 | HIST4H4 | | | | | | |
| IGJ_HUMAN | 4.98 | P01591 | JCHAIN | | | 0.52 | 0.41 | 0.59 | 0.71 |
| CO4A_HUMAN | 4.95 | P0C0L4 | C4A | 0.01 | | | | 0.06 | 0.06 |
| THRB_HUMAN | 4.91 | P00734 | F2 | 0.73 | 0.03 | 0.77 | 0.04 | 0.84 | 1 |
| CERU_HUMAN | 4.89 | P00450 | CP | 0.13 | 0.04 | 0.19 | 0.04 | 0.16 | 0.16 |
| K1C14_HUMAN | 4.83 | P02533 | KRT14 | 0.05 | 0.02 | 0.53 | 0.02 | 0.72 | 0.77 |
| PON1_HUMAN | 4.62 | P27169 | PON1 | 0.26 | 0.05 | 0.13 | 0.05 | 0.29 | 0.29 |
| FA9_HUMAN | 4.59 | P00740 | F9 | 0.68 | | 0.5 | 0.01 | 0.73 | 0.75 |
| CD5L_HUMAN | 4.49 | O43866 | CD5L | 0.03 | | | | 0.1 | 0.11 |
| C4BPB_HUMAN | 4.49 | P20851 | C4BPB | | 0 | | | | 0.05 |
| IC1_HUMAN | 4.41 | P05155 | SERPING1 | 0.68 | 0.28 | 0.54 | 0 | 0.76 | 0.78 |
| CNN2_HUMAN | 4.37 | Q99439 | CNN2 | | 0.06 | 0 | | 0.09 | 0.09 |
| HORN_HUMAN | 4.34 | Q86YZ3 | HRNR | | 0.02 | | | 0.18 | 0.18 |
| CBPB2_HUMAN | 4.22 | Q96IY4 | CPB2 | 0.06 | 0.02 | 0.03 | 0.03 | 0.6 | 0.6 |
| PHLD_HUMAN | 4.22 | P80108 | GPLD1 | 0.02 | | 0 | | 0.08 | 0.09 |
| PROC_HUMAN | 4.16 | P04070 | PROC | 0.7 | | | | 0.77 | 0.77 |
| IGKC_HUMAN | 4.09 | P01834 | IGKC | 0.03 | | 0.02 | | 0.12 | 0.12 |
| CLUS_HUMAN | 4.06 | P10909 | CLU | 0.76 | 0.2 | 0.07 | 0.02 | 0.87 | 0.87 |
| K1C16_HUMAN | 3.93 | P08779 | KRT16 | 0.05 | 0.02 | 0.52 | | 0.72 | 0.75 |
| CRP_HUMAN | 3.9 | P02741 | CRP | 0.22 | 0.07 | 0.05 | 0.05 | 0.31 | 0.32 |
| APOC1_HUMAN | 3.85 | P02654 | APOC1 | 0.06 | 0.03 | 0 | | 0.66 | 0.8 |
| HRG_HUMAN | 3.84 | P04196 | HRG | 0.1 | 0.03 | 0.14 | 0.02 | 0.72 | 0.73 |
| CO8B_HUMAN | 3.76 | P07358 | C8B | 0.54 | | | | 0.68 | 0.68 |
| PROS_HUMAN | 3.73 | P07225 | PROS1 | 0.69 | | 0.71 | | 0.79 | 0.81 |
| APOE_HUMAN | 3.71 | P02649 | APOE | 0.71 | 0.03 | 0.1 | 0.02 | 0.79 | 0.88 |
| A4_HUMAN | 3.7 | P05067 | APP | 0.53 | 0.07 | 0.78 | 0.02 | 0.83 | 0.86 |
| ZYX_HUMAN | 3.69 | Q15942 | ZYX | 0.06 | | 0.03 | | 0.08 | 0.23 |
| ANGT_HUMAN | 3.57 | P01019 | AGT | 0.1 | 0.02 | 0.73 | 0.02 | 0.75 | 0.8 |
| C1QC_HUMAN | 3.56 | P02747 | C1QC | | | | | 0.25 | 0.25 |
| IGHG4_HUMAN | 3.54 | P01861 | IGHG4 | | | | | | 0.02 |
| CO3_HUMAN | 3.51 | P01024 | C3 | 0.02 | 0.04 | 0.7 | | 0.74 | 0.78 |
| KPYM_HUMAN | 3.48 | P14618 | PKM | 0.22 | 0.26 | 0.29 | 0.14 | 0.32 | 0.32 |
| K1C9_HUMAN | 3.45 | P35527 | KRT9 | 0.01 | | 0.52 | | 0.7 | 0.73 |
| APOA_HUMAN | 3.35 | P08519 | LPA | 0.18 | 0.15 | 0.13 | 0.03 | 0.65 | 0.65 |
| GTR1_HUMAN | 3.31 | P11166 | SLC2A1 | 0.19 | 0.14 | 0.07 | 0.04 | 0.29 | 1 |
| ENOA_HUMAN | 3.29 | P06733 | ENO1 | 0.26 | 0.18 | 0.26 | 0.03 | 0.28 | 0.3 |
| LAC2_HUMAN | 3.27 | | | | | | | | |
| VINC_HUMAN | 3.25 | P18206 | VCL | 0.77 | 0.04 | 0.81 | | 0.87 | 1 |
| ITB2_HUMAN | 3.22 | P05107 | ITGB2 | 0 | 0.11 | 0.7 | 0.06 | 0.79 | 0.82 |
| SPTA1_HUMAN | 3.17 | P02549 | SPTA1 | 0.02 | | 0.81 | 0.24 | 0.82 | 0.83 |
| VASP_HUMAN | 3.16 | P50552 | VASP | 0.06 | | 0.59 | | 0.76 | 0.79 |
| K2C1_HUMAN | 3.16 | P04264 | KRT1 | 0.03 | | 0.5 | | 0.75 | 0.76 |
| MASP2_HUMAN | 3.14 | O00187 | MASP2 | | | | | 0.19 | 0.19 |
| HPTR_HUMAN | 3.14 | P00739 | HPR | 0.55 | 0.05 | 0.09 | | 0.72 | 0.73 |
| MYH9_HUMAN | 3.09 | P35579 | MYH9 | 0.62 | 0.35 | 0.61 | | 0.85 | 0.88 |
| APOC3_HUMAN | 3.08 | P02656 | APOC3 | 0.63 | 0.05 | | | 0.69 | 0.69 |
| CD14_HUMAN | 3.06 | P08571 | CD14 | 0.78 | 0.06 | 0.08 | 0.11 | 0.83 | 0.85 |
| ITIH1_HUMAN | 2.88 | P19827 | ITIH1 | 0.01 | | | | 0.01 | 0.01 |
| CXCL7_HUMAN | 2.75 | P02775 | PPBP | 0.06 | 0.03 | 0.7 | 0 | 0.79 | 0.8 |
| DSG1_HUMAN | 2.73 | Q02413 | DSG1 | 0.09 | 0.02 | 0.75 | | 0.81 | 0.82 |
| FHR1_HUMAN | 2.68 | Q03591 | CFHR1 | 0.05 | | 0 | | 0.05 | 0.06 |
| A1BG_HUMAN | 2.65 | P04217 | A1BG | 0.04 | 0.03 | 0.12 | | 0.66 | 0.67 |
| B3AT_HUMAN | 2.63 | P02730 | SLC4A1 | 0.11 | | 0.05 | 0.03 | 0.35 | 0.36 |
| KNG1_HUMAN | 2.62 | P01042 | KNG1 | 0.67 | 0.57 | 0.74 | 0.01 | 0.81 | 0.82 |
| COL10_HUMAN | 2.61 | Q9Y6Z7 | COLEC10 | | | | | 0 | 0.02 |
| K2C5_HUMAN | 2.61 | P13647 | KRT5 | 0.09 | 0.04 | 0.11 | | 1 | 1 |
| A1AT_HUMAN | 2.6 | P01009 | SERPINA1 | 0.17 | 0.07 | 0.14 | 0.04 | 0.73 | 0.74 |
| AACT_HUMAN | 2.54 | P01011 | SERPINA3 | 0.11 | 0.04 | 0.25 | | 0.71 | 0.72 |
| HBD_HUMAN | 2.51 | P02042 | HBD | | 0.01 | 0.01 | | 0.59 | 0.59 |
| BIN2_HUMAN | 2.5 | Q9UBW5 | BIN2 | 0 | | | | 0.02 | 0.02 |
| S10A9_HUMAN | 2.43 | P06702 | S100A9 | 0.07 | 0.04 | 0.11 | 0.04 | 0.62 | 0.63 |
| TSP1_HUMAN | 2.41 | P07996 | THBS1 | 0.1 | 0.1 | 0.14 | 0.04 | 0.84 | 0.84 |
| CALU_HUMAN | 2.4 | O43852 | CALU | 0.07 | 0.02 | 0.01 | | 0.67 | 0.71 |
| FILA2_HUMAN | 2.39 | Q5D862 | FLG2 | | | | | | 0 |
| MYL6_HUMAN | 2.38 | P60660 | MYL6 | 0.01 | | | | 0.53 | 0.53 |
| CFAH_HUMAN | 2.36 | P08603 | CFH | 0.09 | | 0.07 | 0.01 | 0.09 | 0.23 |
| SAA2_HUMAN | 2.35 | P0DJI9 | SAA2 | 0.05 | | | | 0.05 | 0.05 |
| TBB1_HUMAN | 2.33 | Q9H4B7 | TUBB1 | 1 | 1 | 1 | 1 | 1 | 1 |
| ACTN1_HUMAN | 2.31 | P12814 | ACTN1 | 0.01 | | 0.62 | | 0.78 | 0.8 |
| APOL1_HUMAN | 2.3 | O14791 | APOL1 | 0.5 | 0 | | | 0.69 | 0.69 |
| DEF1_HUMAN | 2.29 | P59665 | DEFA1 | | | | | | |
| DEF1_HUMAN | 2.29 | P59665 | DEFA1B | | 0.02 | 0.01 | | 0.22 | 0.24 |
| HPT_HUMAN | 2.28 | P00738 | HP | 0.22 | 0.1 | 0.11 | 0.05 | 0.66 | 0.67 |
| ACTN4_HUMAN | 2.21 | O43707 | ACTN4 | 0.12 | 0.04 | 0.06 | | 0.79 | 0.79 |
| ITAM_HUMAN | 2.19 | P11215 | ITGAM | 0.01 | | 0.63 | | 0.78 | 0.79 |
| ANT3_HUMAN | 2.08 | P01008 | SERPINC1 | 1 | 0.26 | 0.63 | 0.2 | 1 | 1 |
| TMOD3_HUMAN | 2.07 | Q9NYL9 | TMOD3 | 0.5 | | 0.39 | | 0.72 | 0.73 |
| MMRN1_HUMAN | 2.06 | Q13201 | MMRN1 | 0.19 | 0.09 | 0.27 | 0.04 | 0.71 | 0.74 |
| PERM_HUMAN | 2.06 | P05164 | MPO | 0.22 | 0.04 | 0.17 | 0.05 | 0.24 | 0.28 |
| SAA1_HUMAN | 2 | P0DJI8 | SAA1 | 0.27 | 0.04 | 0.05 | 0.03 | 0.82 | 0.82 |
| CFAI_HUMAN | 2 | P05156 | CFI | 0.06 | 0.04 | 0.03 | | 0.08 | 0.23 |
| F13A_HUMAN | 1.97 | P00488 | F13A1 | 0.68 | | 0.72 | | 0.79 | 0.8 |
| ANGL6_HUMAN | 1.96 | Q8NI99 | ANGPTL6 | | | 0.05 | 0.02 | 0.05 | 0.06 |
| TRFL_HUMAN | 1.95 | P02788 | LTF | 0.58 | 0.05 | 0.43 | 0.02 | 0.75 | 0.76 |
| ITA2B_HUMAN | 1.95 | P08514 | ITGA2B | 0.77 | | 0.8 | 0.02 | 0.86 | 1 |
| CFAB_HUMAN | 1.94 | P00751 | CFB | 0.09 | | | | 0.12 | 0.16 |
| TPIS_HUMAN | 1.92 | P60174 | TPI1 | 0.08 | | 0.05 | 0.04 | 0.11 | 0.12 |
| KV206_HUMAN | 1.92 | | | | | | | | |
| HEP2_HUMAN | 1.91 | P05546 | SERPIND1 | 0.43 | | | | 0.73 | 0.75 |
| K22E_HUMAN | 1.91 | P35908 | KRT2 | | | 0.5 | | 0.73 | 0.74 |
| ANK1_HUMAN | 1.9 | P16157 | ANK1 | 0.5 | 0.38 | 0.5 | | 0.85 | 0.87 |
| WDR1_HUMAN | 1.89 | O75083 | WDR1 | | | 0.14 | | 0.67 | 0.68 |
| TPM4_HUMAN | 1.88 | P67936 | TPM4 | 0.68 | | 0 | | 1 | 1 |
| GP1BA_HUMAN | 1.87 | P07359 | GP1BA | 0.67 | | | | 0.76 | 0.81 |
| HSP7C_HUMAN | 1.85 | P11142 | HSPA8 | 0.19 | | 0.04 | 0 | 0.85 | 0.85 |
| QCR2_HUMAN | 1.84 | P22695 | UQCRC2 | 0.01 | 0 | | | 0.01 | 0.01 |
| ECM1_HUMAN | 1.83 | Q16610 | ECM1 | 0.01 | 0 | | | 0.73 | 0.73 |
| ALS_HUMAN | 1.82 | P35858 | IGFALS | 0.11 | | | | 0.64 | 0.64 |
| H2B1C_HUMAN | 1.81 | P62807 | HIST1H2BC | 0.78 | 0 | | | 0.81 | 0.81 |
| H2B1C_HUMAN | 1.81 | P62807 | HIST1H2BE | | | | | 0.38 | 0.38 |
| H2B1C_HUMAN | 1.81 | P62807 | HIST1H2BF | | | | | 0 | 0 |
| H2B1C_HUMAN | 1.81 | P62807 | HIST1H2BG | 0 | | | | 0.01 | 0.01 |
| H2B1C_HUMAN | 1.81 | P62807 | HIST1H2BI | | | | | 0.03 | 0.03 |
| IGHA2_HUMAN | 1.79 | P01877 | IGHA2 | | | | | 0.05 | 0.05 |
| LDHB_HUMAN | 1.77 | P07195 | LDHB | 0.04 | 0.06 | 0.04 | 0.02 | 0.24 | 0.24 |
| FBLN3_HUMAN | 1.77 | Q12805 | EFEMP1 | 0.53 | 0.06 | 0.29 | | 0.7 | 0.7 |
| RAP1B_HUMAN | 1.76 | P61224 | RAP1B | 0.77 | | | | 0.84 | 1 |
| CETP_HUMAN | 1.75 | P11597 | CETP | 0.16 | | 0.03 | 0.01 | 0.63 | 0.75 |
| LBP_HUMAN | 1.74 | P18428 | LBP | 0.6 | | 0.69 | | 0.79 | 0.81 |
| LTBP1_HUMAN | 1.71 | Q14766 | LTBP1 | 0.51 | | 0.03 | | 0.68 | 0.68 |
| HEMO_HUMAN | 1.7 | P02790 | HPX | 0.5 | 0.01 | 0.06 | | 0.67 | 0.67 |
| JAM1_HUMAN | 1.7 | Q9Y624 | F11R | 0.15 | 0.06 | 0.64 | 0.04 | 0.79 | 0.83 |
| ATPB_HUMAN | 1.69 | P06576 | ATP5B | | | | | | |
| STOM_HUMAN | 1.67 | P27105 | STOM | 0.06 | | 0.03 | | 0.27 | 0.27 |
| KLKB1_HUMAN | 1.66 | P03952 | KLKB1 | 0.71 | 0.28 | 0.52 | 0.03 | 0.78 | 0.8 |
| DREB_HUMAN | 1.65 | Q16643 | DBN1 | 0.05 | | 0.07 | | 0.24 | 0.25 |
| C1QB_HUMAN | 1.64 | P02746 | C1QB | | | 0 | | 0.26 | 0.26 |
| APOC2_HUMAN | 1.63 | P02655 | APOC2 | | 0.04 | 0.04 | | 0.05 | 0.06 |
| K1C10_HUMAN | 1.63 | P13645 | KRT10 | 0.03 | 0.51 | | | 0.74 | 0.74 |
| AT2A2_HUMAN | 1.6 | P16615 | ATP2A2 | 0.64 | 0.28 | 0.07 | | 0.8 | 0.8 |
| SPTB1_HUMAN | 1.59 | P11277 | SPTB | | | 0.8 | | 0.82 | 0.83 |
| APOC4_HUMAN | 1.58 | P55056 | APOC4 | 0.63 | | | | 0.72 | 0.72 |
| CH60_HUMAN | 1.56 | P10809 | HSPD1 | 0.22 | 0.02 | 0.06 | 0.03 | 0.31 | 0.31 |
| CPT1A_HUMAN | 1.56 | P50416 | CPT1A | | | 0.03 | | 0.26 | 0.28 |
| EMIL1_HUMAN | 1.56 | Q9Y6C2 | EMILIN1 | | | | | 0.02 | 0.15 |
| APOA4_HUMAN | 1.54 | P06727 | APOA4 | 0.61 | 0.05 | | 0.02 | 0.79 | 0.79 |
| 1433F_HUMAN | 1.54 | Q04917 | YWHAH | | | 0.77 | | 0.79 | 0.81 |
| APOM_HUMAN | 1.54 | O95445 | APOM | 0.94 | | | | 1 | 1 |
| KV205_HUMAN | 1.51 | | | | | | | | |
| ARF1_HUMAN | 1.51 | P84077 | ARF1 | | | 0.03 | | 0.75 | 0.75 |
| HV209_HUMAN | 1.51 | | | | | | | | |
| CO2_HUMAN | 1.51 | P06681 | C2 | | | 0 | | 0.05 | 0.06 |
| ITB1_HUMAN | 1.51 | P05556 | ITGB1 | 0.84 | 0.82 | 0.77 | 0.11 | 0.92 | 0.92 |
| AMBP_HUMAN | 1.48 | P02760 | AMBP | 0.51 | | | | 0.69 | 0.69 |
| HV306_HUMAN | 1.47 | | | | | | | | |
| ATPA_HUMAN | 1.47 | P25705 | ATP5A1 | | | | | | |
| SBSN_HUMAN | 1.44 | Q6UWP8 | SBSN | 0.06 | | 0.01 | | 0.1 | 0.1 |
| PRG4_HUMAN | 1.4 | Q92954 | PRG4 | | | | | 0.07 | 0.08 |
| TLN1_HUMAN | 1.39 | Q9Y490 | TLN1 | 0.76 | | 0.04 | | 0.89 | 1 |
| FLNA_HUMAN | 1.36 | P21333 | FLNA | 0.44 | 0.29 | 0.72 | | 0.88 | 0.91 |
| S10A8_HUMAN | 1.35 | P05109 | S100A8 | 0.42 | 0.08 | 0.61 | | 0.71 | 0.8 |
| SPP24_HUMAN | 1.35 | Q13103 | SPP2 | 0.03 | 0.03 | 0.12 | | 0.67 | 0.67 |
| PPIA_HUMAN | 1.34 | P62937 | PPIA | 0.05 | 0.05 | 0.04 | 0.32 | 0.96 | 1 |
| PPIF_HUMAN | 1.34 | P30405 | PPIF | 0.04 | 0.04 | 0.04 | | 0.08 | 0.08 |
| FA12_HUMAN | 1.34 | P00748 | F12 | 0.68 | 0.02 | 0.06 | 0.02 | 0.73 | 0.73 |
| MOES_HUMAN | 1.34 | P26038 | MSN | 0.8 | 0.14 | 0.82 | | 1 | 1 |
| G3P_HUMAN | 1.34 | P04406 | GAPDH | 0.18 | 0.06 | 0.2 | 0.05 | 0.19 | 0.23 |
| CD59_HUMAN | 1.32 | P13987 | CD59 | 0.06 | 0.02 | 0.07 | 0.02 | 0.28 | 0.28 |
| CALX_HUMAN | 1.32 | P27824 | CANX | 0.05 | | 0.05 | 0.03 | 0.75 | 0.75 |
| A2AP_HUMAN | 1.32 | P08697 | SERPINF2 | 0.05 | 0.04 | 0.04 | 0.02 | 0.66 | 0.67 |
| ITIH3_HUMAN | 1.32 | Q06033 | ITIH3 | 0.01 | 0.02 | 0.12 | | 0.67 | 0.68 |
| AOFB_HUMAN | 1.31 | P27338 | MAOB | 0.13 | | 0.3 | 0.02 | 0.28 | 0.37 |
| PLF4_HUMAN | 1.31 | P02776 | PF4 | 0.42 | 0.05 | 0.21 | 0.17 | 0.74 | 0.79 |
| GPV_HUMAN | 1.31 | P40197 | GP5 | 0.67 | | | 0.02 | 0.74 | 0.74 |
| AFAM_HUMAN | 1.31 | P43652 | AFM | | 0.01 | 0.03 | 0.01 | 0.56 | 0.57 |
| PDLI1_HUMAN | 1.3 | O00151 | PDLIM1 | | 0.04 | 0.13 | | 0.21 | 0.24 |
| DCD_HUMAN | 1.29 | P81605 | DCD | 0.5 | 0.06 | | | 0.73 | 0.73 |
| ENPL_HUMAN | 1.29 | P14625 | HSP90B1 | 0.7 | 0.06 | 0.07 | 0.03 | 0.82 | 0.82 |
| TBB5_HUMAN | 1.27 | P07437 | TUBB | 1 | 1 | 1 | 1 | 1 | 1 |
| PGH1_HUMAN | 1.27 | P23219 | PTGS1 | 1 | 0.04 | 0.28 | 0.93 | 1 | 1 |
| 1A02_HUMAN | 1.27 | P01892 | HLA-A | 0.87 | | 0.58 | | 1 | 1 |
| ARP3_HUMAN | 1.22 | P61158 | ACTR3 | | | 0.07 | | 0.04 | 0.07 |
| RTN4_HUMAN | 1.22 | Q9NQC3 | RTN4 | 0.18 | | 0 | | 0.56 | 0.56 |
| CRAC1_HUMAN | 1.21 | Q9NQ79 | CRTAC1 | 0.31 | | | | 0.31 | 0.31 |
| AT2A3_HUMAN | 1.2 | Q93084 | ATP2A3 | 0.63 | | 0.62 | | 0.8 | 0.81 |
| COR1C_HUMAN | 1.2 | Q9ULV4 | CORO1C | 0.03 | | 0.16 | | 0.25 | 0.26 |
| KAIN_HUMAN | 1.2 | P29622 | SERPINA4 | 0.06 | | 0.03 | 0.01 | 0.67 | 0.67 |
| ITB3_HUMAN | 1.19 | P05106 | ITGB3 | 0.84 | 0.04 | 1 | | 0.92 | 1 |
| PEDF_HUMAN | 1.19 | P36955 | SERPINF1 | 0.1 | 0.16 | 0.2 | 0.08 | 0.3 | 0.31 |
| APOF_HUMAN | 1.18 | Q13790 | APOF | 0.16 | | 0.03 | | 0.55 | 0.56 |
| TBA4A_HUMAN | 1.17 | P68366 | TUBA4A | 1 | 1 | 1 | 1 | 1 | 1 |
| PGK1_HUMAN | 1.17 | P00558 | PGK1 | 0.04 | 0.03 | 0.12 | | 0.24 | 0.25 |
| TTHY_HUMAN | 1.16 | P02766 | TTR | 0.57 | 0.06 | 0.1 | 0.05 | 0.8 | 0.8 |
| STXB2_HUMAN | 1.15 | Q15833 | STXBP2 | | | 0.12 | | 0.65 | 0.67 |
| LG3BP_HUMAN | 1.14 | Q08380 | LGALS3BP | 0.06 | 0.06 | 0 | | 0.73 | 0.73 |
| MYL9_HUMAN | 1.13 | P24844 | MYL9 | 0.51 | 0.02 | 0.08 | | 0.64 | 0.65 |
| CAP1_HUMAN | 1.13 | Q01518 | CAP1 | | 0.03 | 0 | | 0.67 | 0.67 |
| SHBG_HUMAN | 1.12 | P04278 | SHBG | 0.04 | 0.03 | 0.03 | 0.01 | 0.24 | 0.24 |
| BAP31_HUMAN | 1.11 | P51572 | BCAP31 | 0.03 | 0.01 | | | 0.73 | 0.73 |
| LUM_HUMAN | 1.11 | P51884 | LUM | 0.7 | 0.1 | 0.07 | | 0.84 | 0.84 |
| QSOX1_HUMAN | 1.11 | O00391 | QSOX1 | 0 | 0.08 | 0.11 | | 0.71 | 0.71 |
| RSU1_HUMAN | 1.1 | Q15404 | RSU1 | | | 0.36 | | 0.53 | 0.62 |
| CYTA_HUMAN | 1.09 | P01040 | CSTA | 0.06 | 0 | 0.63 | | 0.13 | 0.66 |
| CAPZB_HUMAN | 1.06 | P47756 | CAPZB | | | | | 0.51 | 0.51 |
| THAS_HUMAN | 1.05 | P24557 | TBXAS1 | 0.15 | | 0.26 | | 0.61 | 0.67 |
| LYSC_HUMAN | 1.03 | P61626 | LYZ | | 0 | 0 | 0.02 | 0.39 | 0.39 |
| FHR2_HUMAN | 1.02 | P36980 | CFHR2 | | | | | 0.02 | 0.02 |
| RAB8A_HUMAN | 1.02 | P61006 | RAB8A | 0.08 | 0.01 | 0.66 | 0.03 | 0.21 | 0.68 |
| TSP4_HUMAN | 1.02 | P35443 | THBS4 | | | 0.01 | | 0.22 | 0.22 |
| 1433Z_HUMAN | 1.02 | P63104 | YWHAZ | 0.06 | | 0.02 | | 0.86 | 0.86 |
| A1AG1_HUMAN | 1.01 | P02763 | ORM1 | 0.03 | 0 | | | 0.17 | 0.17 |
| AMY1_HUMAN | 1 | P04745 | AMY1A | 0.5 | | | | 0.5 | 0.5 |
| AMY1_HUMAN | 1 | P04745 | AMY1B | 0.5 | | | | 0.63 | 0.63 |
| AMY1_HUMAN | 1 | P04745 | AMY1C | | | | 0.02 | 0.05 | 0.06 |
| KRT86_HUMAN | 1 | O43790 | KRT86 | | | 0.52 | | 0.69 | 0.72 |
| DESP_HUMAN | 1 | P15924 | DSP | 0.1 | | 0.76 | | 0.82 | 0.83 |
| K2C6A_HUMAN | 0.99 | P02538 | KRT6A | 0.02 | 0 | | | 0.75 | 0.75 |
| HV303_HUMAN | 0.99 | | | | | | | | |
| URP2_HUMAN | 0.98 | Q86UX7 | FERMT3 | 0.04 | | 0.05 | | 0.71 | 0.73 |
| A2GL_HUMAN | 0.98 | P02750 | LRG1 | 0.06 | 0.08 | 0.08 | | 0.28 | 0.28 |
| NEXN_HUMAN | 0.98 | Q0ZGT2 | NEXN | 0 | | 0 | | 0 | 0.02 |
| KV105_HUMAN | 0.97 | P01602 | IGKV1-5 | | | | | | 0.02 |
| CALL5_HUMAN | 0.96 | Q9NZT1 | CALML5 | | | | | 0.06 | 0.06 |
| CALR_HUMAN | 0.94 | P27797 | CALR | 0.9 | 0.57 | 0.18 | | 1 | 1 |
| GNAI2_HUMAN | 0.93 | P04899 | GNAI2 | 0.01 | | 0.02 | | 0.8 | 0.85 |
| CEAM8_HUMAN | 0.93 | P31997 | CEACAM8 | 0.03 | | | | 0.65 | 0.65 |
| PGRP2_HUMAN | 0.93 | Q96PD5 | PGLYRP2 | 0.56 | | 0.41 | | 0.69 | 0.71 |
| K2C6C_HUMAN | 0.93 | P48668 | KRT6C | | | 0.1 | | 0.76 | 0.76 |
| FBLN1_HUMAN | 0.92 | P23142 | FBLN1 | 0.52 | 0.02 | 0.03 | | 0.72 | 0.74 |
| ITIH4_HUMAN | 0.92 | Q14624 | ITIH4 | 0.01 | 0.28 | 0.14 | 0.02 | 0.7 | 0.7 |
| THBG_HUMAN | 0.92 | P05543 | SERPINA7 | | | | 0.03 | 0.12 | 0.12 |
| IGHM_HUMAN | 0.92 | P01871 | IGHM | | 0.01 | | | 0.05 | 0.12 |
| TAGL2_HUMAN | 0.91 | P37802 | TAGLN2 | 0.04 | 0.05 | 0.23 | | 0.67 | 0.71 |
| LIMS1_HUMAN | 0.9 | P48059 | LIMS1 | | | 0.19 | | 0.69 | 0.73 |
| MBL2_HUMAN | 0.87 | P11226 | MBL2 | 0.07 | 0.1 | 0.1 | 0.04 | 0.22 | 0.35 |
| EFTU_HUMAN | 0.86 | P49411 | TUFM | 0.05 | | | | 0.17 | 0.18 |
| PPIP2_HUMAN | 0.86 | Q9H939 | PSTPIP2 | | | 0 | | | 0 |
| K1C17_HUMAN | 0.84 | Q04695 | KRT17 | 0.02 | 0.03 | 0.52 | | 0.67 | 0.73 |
| PRDX2_HUMAN | 0.83 | P32119 | PRDX2 | 0.55 | 0.13 | 0.09 | 0.05 | 0.72 | 0.72 |
| CAH1_HUMAN | 0.83 | P00915 | CA1 | 0.09 | 0.05 | 0.06 | 0.02 | 0.38 | 0.38 |
| CAN1_HUMAN | 0.81 | P07384 | CAPN1 | 0.08 | | 0.63 | | 0.76 | 0.82 |
| SAMP_HUMAN | 0.8 | P02743 | APCS | 0.04 | 0.01 | | 0.03 | 0.04 | 0.05 |
| EPB42_HUMAN | 0.8 | P16452 | EPB42 | | | | | | 0.05 |
| PON3_HUMAN | 0.8 | Q15166 | PON3 | | | 0 | | 0.24 | 0.24 |
| RB27B_HUMAN | 0.77 | O00194 | RAB27B | 0 | 0.1 | 0.01 | | 0.23 | 0.23 |
| TBB4B_HUMAN | 0.76 | P68371 | TUBB4B | 1 | 1 | 1 | 1 | 1 | 1 |
| CO6A3_HUMAN | 0.76 | P12111 | COL6A3 | 0.21 | 0.7 | 0.5 | 0.13 | 0.79 | 0.8 |
| KRT82_HUMAN | 0.76 | Q9NSB4 | KRT82 | | | | | 0.73 | 0.73 |
| TCPG_HUMAN | 0.75 | P49368 | CCT3 | 0.72 | | 0.01 | 0.07 | 0.86 | 0.86 |
| GTR3_HUMAN | 0.75 | P11169 | SLC2A3 | 0.02 | 0 | 0.07 | | 0.04 | 0.13 |
| ACTB_HUMAN | 0.74 | P60709 | ACTB | 0.04 | 0.02 | 0.08 | 0.15 | 0.86 | 1 |
| ACTC_HUMAN | 0.73 | P68032 | ACTC1 | | | 0.06 | | 0.18 | 0.2 |
| MYLK_HUMAN | 0.73 | Q15746 | MYLK | 0.06 | | 0.01 | | 0.49 | 0.5 |
| SPRL1_HUMAN | 0.72 | Q14515 | SPARCL1 | 0.06 | 0.05 | 0.05 | | 0.26 | 0.27 |
| ILK_HUMAN | 0.71 | Q13418 | ILK | 0.19 | 0.07 | 0.22 | 0.04 | 0.73 | 0.74 |
| TYB4_HUMAN | 0.71 | P62328 | TMSB4X | 0.07 | 0.07 | 0.06 | 0.06 | 0.13 | 0.19 |
| NP1L1_HUMAN | 0.7 | P55209 | NAP1L1 | 0 | | 0.04 | | 0.2 | 0.2 |
| PDIA1_HUMAN | 0.7 | P07237 | P4HB | 0.69 | 0.02 | 0.24 | 0.04 | 0.83 | 0.84 |
| CBPQ_HUMAN | 0.7 | Q9Y646 | CPQ | 0.02 | 0.01 | 0 | | 0.13 | 0.14 |
| PLEK_HUMAN | 0.69 | P08567 | PLEK | 0.01 | 0.02 | 0.03 | | 0.67 | 0.67 |
| SYUA_HUMAN | 0.69 | P37840 | SNCA | 0.04 | | 0.05 | | 0.08 | 0.09 |
| A1AG2_HUMAN | 0.69 | P19652 | ORM2 | | 0 | | | 0.7 | 0.7 |
| C1QA_HUMAN | 0.69 | P02745 | C1QA | | | 0 | | 0.26 | 0.26 |
| DKK3_HUMAN | 0.68 | Q9UBP4 | DKK3 | 0.23 | 0.23 | 0.23 | 0.01 | 0.3 | 0.36 |
| IDHP_HUMAN | 0.66 | P48735 | IDH2 | 0.77 | | 1 | 0.76 | 1 | 1 |
| CO6A2_HUMAN | 0.66 | P12110 | COL6A2 | 0.21 | 0.01 | 0.5 | | 0.75 | 0.76 |
| B2MG_HUMAN | 0.66 | P61769 | B2M | 0.94 | 0.02 | 0.04 | 0.44 | 1 | 1 |
| LYAM3_HUMAN | 0.66 | P16109 | SELP | 0.07 | 0.03 | 0.58 | 0.05 | 0.72 | 0.78 |
| CLIC1_HUMAN | 0.66 | O00299 | CLIC1 | 0.06 | 0.05 | 0.27 | | 0.15 | 0.33 |
| KV312_HUMAN | 0.65 | | | | | | | | |
| ITLN1_HUMAN | 0.65 | Q8WWA0 | ITLN1 | 0.58 | 0.03 | 0.41 | | 0.74 | 0.77 |
| APOA5_HUMAN | 0.65 | Q6Q788 | APOA5 | 0.32 | | 0.03 | | 0.59 | 0.6 |
| STX11_HUMAN | 0.65 | O75558 | STX11 | 0 | | | | 0 | 0.09 |
| PROZ_HUMAN | 0.65 | P22891 | PROZ | | | | | 0.35 | 0.35 |
| GP1BB_HUMAN | 0.64 | P13224 | GP1BB | | | | | 0.01 | 0.22 |
| PGRP1_HUMAN | 0.64 | O75594 | PGLYRP1 | 0.56 | | | | 0.69 | 0.69 |
| HV320_HUMAN | 0.63 | A0A0C4DH32 | IGHV3-20 | | | | | | |
| TBA1B_HUMAN | 0.63 | P68363 | TUBA1B | 1 | 1 | 1 | 1 | 1 | 1 |
| NID1_HUMAN | 0.63 | P14543 | NID1 | 0.51 | | 0.03 | | 0.76 | 0.77 |
| K2C4_HUMAN | 0.62 | P19013 | KRT4 | 0.03 | 0.02 | 0.5 | 0.02 | 0.76 | 0.77 |
| APMAP_HUMAN | 0.62 | Q9HDC9 | APMAP | | | | | | 0 |
| CNDP1_HUMAN | 0.62 | Q96KN2 | CNDP1 | 0.02 | 0.01 | 0.02 | | 0.02 | 0.08 |
| CAP7_HUMAN | 0.61 | P20160 | AZU1 | 0.01 | | | 0.03 | 0.04 | 0.05 |
| C1RL_HUMAN | 0.61 | Q9NZP8 | C1RL | | | 0 | | 0.02 | 0.03 |
| ENDD1_HUMAN | 0.61 | O94919 | ENDOD1 | 0 | | 0 | | 0.66 | 0.66 |
| SYWC_HUMAN | 0.6 | P23381 | WARS | | | 0 | | 0 | 0.02 |
| CMGA_HUMAN | 0.6 | P10645 | CHGA | 0.58 | 0.11 | 0.06 | | 0.63 | 0.63 |
| MPCP_HUMAN | 0.6 | Q00325 | SLC25A3 | | 0.01 | 0 | | 0.1 | 0.14 |
| ELNE_HUMAN | 0.59 | P08246 | ELANE | 0.69 | 0.03 | 0.43 | 0.01 | 0.77 | 0.78 |
| ALDOA_HUMAN | 0.58 | P04075 | ALDOA | 0.11 | 0.05 | | | 0.69 | 0.7 |
| ITA6_HUMAN | 0.58 | P23229 | ITGA6 | 0.51 | 0.06 | 0.05 | 0.01 | 0.98 | 0.98 |
| ARP2_HUMAN | 0.57 | P61160 | ACTR2 | 0.24 | | 0.01 | | 0.58 | 0.58 |
| KV101_HUMAN | 0.57 | | | | | | | | |
| KIF2A_HUMAN | 0.56 | O00139 | KIF2A | 0.18 | 0.04 | 0.24 | 0.03 | 0.69 | 0.7 |
| SAC1_HUMAN | 0.56 | Q9NTJ5 | SACM1L | 0.17 | | | | 0.69 | 0.7 |
| PSB8_HUMAN | 0.55 | P28062 | PSMB8 | 0.85 | 0.27 | 1 | 1 | 1 | 1 |
| CAMP_HUMAN | 0.55 | P49913 | CAMP | 0.51 | | | | 0.64 | 0.64 |
| TPM3_HUMAN | 0.55 | P06753 | TPM3 | 0.61 | | 0.47 | | 0.87 | 0.88 |
| SPRC_HUMAN | 0.54 | P09486 | SPARC | 0.54 | 0.18 | 0.26 | 0.01 | 0.78 | 0.78 |
| HS90A_HUMAN | 0.54 | P07900 | HSP90AA1 | 1 | 0.25 | 0.61 | 0.77 | 1 | 1 |
| CO6A1_HUMAN | 0.54 | P12109 | COL6A1 | 0.21 | 0.01 | 0.51 | | 0.67 | 0.73 |
| PSA7_HUMAN | 0.54 | O14818 | PSMA7 | 0.85 | 0.27 | 0.79 | 1 | 1 | 1 |
| IMB1_HUMAN | 0.53 | Q14974 | KPNB1 | 0.04 | 0.63 | 0.63 | | 0.81 | 0.82 |
| DEMA_HUMAN | 0.53 | Q08495 | DMTN | | | 0.02 | | 0.06 | 0.06 |
| 41_HUMAN | 0.53 | P11171 | EPB41 | 0.06 | | 0.06 | | 0.07 | 0.07 |
| GPIX_HUMAN | 0.53 | P14770 | GP9 | 0.39 | | | | 0.57 | 0.61 |
| FETUB_HUMAN | 0.52 | Q9UGM5 | FETUB | | | | | 0.02 | 0.02 |
| GPDM_HUMAN | 0.52 | P43304 | GPD2 | 0.32 | 0.3 | 0.79 | 0.2 | 1 | 1 |
| SYTL4_HUMAN | 0.52 | Q96C24 | SYTL4 | | | 0.12 | | 0.65 | 0.66 |
| PDIA6_HUMAN | 0.52 | Q15084 | PDIA6 | 0.03 | | 0.04 | | 0.05 | 0.07 |
| PHB2_HUMAN | 0.52 | Q99623 | PHB2 | | 0.05 | | | 0.24 | 0.24 |
| H2A2A_HUMAN | 0.51 | Q6FI13 | HIST2H2AA3 | | | | | | 0.01 |
| H2A2A_HUMAN | 0.51 | Q6FI13 | HIST2H2AA4 | | | | | | |
| ANXA2_HUMAN | 0.51 | P07355 | ANXA2 | 0.12 | 0.19 | 0.23 | 0.08 | 0.31 | 0.31 |
| IPSP_HUMAN | 0.51 | P05154 | SERPINA5 | 0.76 | 0.03 | 0.68 | 0.02 | 0.81 | 0.84 |
| RDH11_HUMAN | 0.5 | Q8TC12 | RDH11 | 0.54 | | | | 0.67 | 0.67 |
| TSP2_HUMAN | 0.5 | P35442 | THBS2 | 0.13 | 0.04 | 0.06 | 0.02 | 0.79 | 0.79 |
| PSA6_HUMAN | 0.5 | P60900 | PSMA6 | 0.87 | 0.27 | 0.79 | 1 | 1 | 1 |
| KPCB_HUMAN | 0.5 | P05771 | PRKCB | 0.52 | 0.81 | 0.97 | 0.21 | 0.96 | 1 |
| SIAT1_HUMAN | 0.49 | P15907 | ST6GAL1 | 0.35 | 0.03 | 0.36 | | 0.84 | 0.86 |
| H13_HUMAN | 0.49 | P16402 | HIST1H1D | 0.71 | | | 0 | 0.79 | 0.79 |
| GPX3_HUMAN | 0.49 | P22352 | GPX3 | 0.19 | | 0.01 | 0.02 | 0.3 | 0.35 |
| K1C13_HUMAN | 0.49 | P13646 | KRT13 | 0.03 | 0.51 | 0.51 | | 0.78 | 0.8 |
| ECHA_HUMAN | 0.49 | P40939 | HADHA | 0.04 | | 0.5 | | 0.16 | 0.54 |
| DEF3_HUMAN | 0.48 | P59666 | DEFA3 | | | | | 0.58 | 0.58 |
| ADT2_HUMAN | 0.47 | P05141 | SLC25A5 | | | | | 0.43 | 0.43 |
| TBA8_HUMAN | 0.47 | Q9NY65 | TUBA8 | 0.66 | | 0.63 | | 0.78 | 0.79 |
| CSPG2_HUMAN | 0.47 | P13611 | VCAN | 0.05 | 0.51 | 0.04 | 0.03 | 0.84 | 0.84 |
| SRCRL_HUMAN | 0.47 | A1L4H1 | SSC5D | 0.22 | | | | 0.22 | 0.22 |
| GGCT_HUMAN | 0.47 | O75223 | GGCT | 0.04 | | 0.07 | | 0.22 | 0.25 |
| CAZA1_HUMAN | 0.47 | P52907 | CAPZA1 | | | | | 0.53 | 0.54 |
| TOR4A_HUMAN | 0.47 | Q9NXH8 | TOR4A | | | | | | |
| RPN1_HUMAN | 0.46 | P04843 | RPN1 | 0.02 | | | | 0.06 | 0.07 |
| FABP5_HUMAN | 0.46 | Q01469 | FABP5 | 0.08 | | 0.15 | | 0.27 | 0.27 |
| CLH1_HUMAN | 0.45 | Q00610 | CLTC | 0.75 | 0.01 | 0.77 | 0.28 | 0.94 | 0.95 |
| TERA_HUMAN | 0.45 | P55072 | VCP | 0.69 | 0.04 | 0.66 | 0.02 | 0.85 | 0.86 |
| ANXA7_HUMAN | 0.45 | P20073 | ANXA7 | 0.02 | 0.04 | 0.13 | 0.04 | 0.2 | 0.26 |
| ROCK2_HUMAN | 0.45 | O75116 | ROCK2 | 0.53 | 0.07 | 0.15 | 0.09 | 0.78 | 0.78 |
| CD36_HUMAN | 0.45 | P16671 | CD36 | 0.68 | 0 | 0.71 | 0.03 | 0.83 | 0.86 |
| LV102_HUMAN | 0.45 | | | | | | | | |
| PDIA3_HUMAN | 0.45 | P30101 | PDIA3 | 0.06 | | 0.07 | | 0.26 | 0.28 |
| LYAM1_HUMAN | 0.44 | P14151 | SELL | 0.05 | 0.04 | 0.02 | 0.03 | 0.52 | 0.53 |
| KRT85_HUMAN | 0.44 | P78386 | KRT85 | | | | | 0.74 | 0.74 |
| GRP75_HUMAN | 0.44 | P38646 | HSPA9 | 0.1 | 0.04 | 0.03 | 0.03 | 0.55 | 0.55 |
| HV305_HUMAN | 0.44 | | | | | | | | |
| VDAC3_HUMAN | 0.44 | Q9Y277 | VDAC3 | | 0.02 | 0.63 | | 0.05 | 0.64 |
| PTX3_HUMAN | 0.44 | P26022 | PTX3 | 0 | 0 | 0 | | 0.02 | 0.02 |
| FILA_HUMAN | 0.43 | P20930 | FLG | 0.09 | 0.5 | 0.64 | | 0.82 | 0.83 |
| SRC_HUMAN | 0.43 | P12931 | SRC | 1 | 0.32 | 0.99 | 0.3 | 1 | 1 |
| VDAC1_HUMAN | 0.43 | P21796 | VDAC1 | 0.23 | 0.12 | 0.66 | 0.04 | 0.28 | 0.7 |
| VDAC2_HUMAN | 0.43 | P45880 | VDAC2 | 0.02 | 0.7 | 0.02 | | 0.75 | 0.75 |
| GNAZ_HUMAN | 0.43 | P19086 | GNAZ | 0.63 | | 0.01 | | 0.78 | 0.81 |
| SORCN_HUMAN | 0.42 | P30626 | SRI | 0.06 | | 0.04 | 0.04 | 0.23 | 0.24 |
| CY24B_HUMAN | 0.42 | P04839 | CYBB | 0.05 | 0.02 | 0.48 | 0.09 | 0.69 | 0.73 |
| PCBP1_HUMAN | 0.42 | Q15365 | PCBP1 | 0.36 | 0.4 | | | 0.52 | 0.53 |
| PAFA_HUMAN | 0.42 | Q13093 | PLA2G7 | 0.02 | | | 0.01 | 0.26 | 0.27 |
| TENX_HUMAN | 0.41 | P22105 | TNXB | 0.88 | | 0.06 | 0 | 1 | 1 |
| MDHM_HUMAN | 0.4 | P40926 | MDH2 | 0.04 | | 0.02 | | 0.18 | 0.18 |
| PERE_HUMAN | 0.4 | P11678 | EPX | 0.01 | | | | 0.04 | 0.17 |
| ANXA1_HUMAN | 0.4 | P04083 | ANXA1 | 0.24 | 0.08 | 0.56 | | 0.84 | 0.86 |
| TRML1_HUMAN | 0.4 | Q86YW5 | TREML1 | | | | | | 0.02 |
| PRTN3_HUMAN | 0.4 | P24158 | PRTN3 | 0.06 | | 0.16 | | 0.64 | 0.67 |
| S10AC_HUMAN | 0.4 | P80511 | S100A12 | 0.05 | 0.05 | 0.03 | | 0.52 | 0.52 |
| SVEP1_HUMAN | 0.39 | Q4LDE5 | SVEP1 | 0.14 | | | | 0.21 | 0.21 |
| PHB_HUMAN | 0.38 | P35232 | PHB | 0.14 | 0.12 | 0.06 | | 0.78 | 0.78 |
| HSP71_HUMAN | 0.38 | | | | | | | | |
| MAOM_HUMAN | 0.38 | P23368 | ME2 | 0.05 | 0.04 | 0.18 | 0.02 | 0.08 | 0.19 |
| PLAK_HUMAN | 0.38 | P14923 | JUP | 0.08 | 0 | | | 0.8 | 0.8 |
| GNAQ_HUMAN | 0.37 | P50148 | GNAQ | 0.81 | 0.01 | 0.71 | | 1 | 1 |
| SRC8_HUMAN | 0.37 | Q14247 | CTTN | 0.15 | | 0.51 | | 0.16 | 0.55 |
| ZO2_HUMAN | 0.37 | Q9UDY2 | TJP2 | 0.14 | 0.04 | 0.02 | | 0.82 | 0.82 |
| THTR_HUMAN | 0.37 | Q16762 | TST | 0.03 | | 0.02 | | 0.13 | 0.14 |
| LV301_HUMAN | 0.36 | P01715 | IGLV3-1 | | | | | | |
| ATPD_HUMAN | 0.36 | P30049 | ATP5D | | | | | | |
| MARCO_HUMAN | 0.36 | Q9UEW3 | MARCO | 0.68 | | 0.3 | | 0.71 | 0.72 |
| CATA_HUMAN | 0.36 | P04040 | CAT | 0.16 | 0.07 | 0.2 | 0.04 | 0.69 | 0.7 |
| DSC1_HUMAN | 0.36 | Q08554 | DSC1 | 0.08 | | 0.63 | | 0.74 | 0.77 |
| TPM2_HUMAN | 0.36 | P07951 | TPM2 | 0.5 | 0 | | | 0.75 | 0.75 |
| FA8_HUMAN | 0.35 | P00451 | F8 | 0.72 | 0.54 | 0.64 | 0.01 | 0.8 | 0.81 |
| S10A7_HUMAN | 0.35 | P31151 | S100A7 | 0.61 | 0.19 | 0.28 | | 0.7 | 0.7 |
| NEUG_HUMAN | 0.35 | Q92686 | NRGN | 0.01 | | 0.02 | | 0.02 | 0.04 |
| ML12A_HUMAN | 0.35 | P19105 | MYL12A | 0.03 | 0 | 0.01 | | 0.48 | 0.48 |
| HXK1_HUMAN | 0.35 | P19367 | HK1 | 0.06 | | 0.06 | 0.02 | 0.15 | 0.15 |
| CD9_HUMAN | 0.35 | P21926 | CD9 | 0.23 | 0.06 | 0.22 | 0.1 | 0.79 | 0.81 |
| LV302_HUMAN | 0.35 | | | | | | | | |
| CLC1B_HUMAN | 0.34 | Q9P126 | CLEC1B | 0.01 | | | | 0.62 | 0.66 |
| HPSE_HUMAN | 0.34 | Q9Y251 | HPSE | 0.27 | 0.14 | 0.11 | 0.26 | 1 | 1 |
| SODM_HUMAN | 0.34 | P04179 | SOD2 | 0.09 | 0.1 | 0.07 | 0.05 | 0.25 | 0.25 |
| PLTP_HUMAN | 0.34 | P55058 | PLTP | 0 | | 0.08 | | 0.42 | 0.44 |
| ATL2_HUMAN | 0.34 | Q86TH1 | ADAMTSL2 | | | | | 0.38 | 0.38 |
| TGFB1_HUMAN | 0.34 | P01137 | TGFB1 | 0.59 | 0.23 | 0.3 | 0.06 | 0.88 | 1 |
| TENA_HUMAN | 0.33 | P24821 | TNC | 0.47 | 0.41 | 0.33 | 0.28 | 0.79 | 0.8 |
| SEP11_HUMAN | 0.33 | Q9NVA2 | SEP11 | | | 0 | | 0.28 | 0.29 |
| KV404_HUMAN | 0.32 | | | | | | | | |
| PRG2_HUMAN | 0.32 | P13727 | PRG2 | | | 0.01 | | 0.03 | 0.03 |
| ECH1_HUMAN | 0.32 | Q13011 | ECH1 | 0.03 | | | | 0.03 | 0.03 |
| 1433E_HUMAN | 0.32 | P62258 | YWHAE | 0.28 | 0.35 | 0.83 | | 0.93 | 0.93 |
| F13B_HUMAN | 0.32 | P05160 | F13B | 0.68 | | 0.63 | | 0.74 | 0.77 |
| PRDX6_HUMAN | 0.32 | P30041 | PRDX6 | 0.17 | 0.01 | 0.04 | | 0.53 | 0.53 |
| BPI_HUMAN | 0.31 | P17213 | BPI | 0.56 | | 0.41 | | 0.69 | 0.72 |
| SDPR_HUMAN | 0.31 | | | | | | | | |
| FHL1_HUMAN | 0.31 | Q13642 | FHL1 | 0.09 | | 0.02 | | 0.21 | 0.22 |
| PCYOX_HUMAN | 0.31 | Q9UHG3 | PCYOX1 | | | | | 0 | 0.01 |
| RAB14_HUMAN | 0.31 | P61106 | RAB14 | 0.19 | | | | 0.68 | 0.68 |
| DECR_HUMAN | 0.31 | Q16698 | DECR1 | | | | | | 0 |
| K2C6B_HUMAN | 0.31 | P04259 | KRT6B | 0 | 0 | 0.52 | | 0.74 | 0.76 |
| MYPT1_HUMAN | 0.31 | O14974 | PPP1R12A | | 0.01 | | | 0.72 | 0.72 |
| PGCA_HUMAN | 0.3 | P16112 | ACAN | 0.68 | 0.5 | 0 | | 0.78 | 0.79 |
| BGH3_HUMAN | 0.3 | Q15582 | TGFBI | 0.25 | 0.07 | 0.13 | | 0.3 | 0.3 |
| FA11_HUMAN | 0.3 | P03951 | F11 | 0.69 | 0.04 | 0.05 | 0.04 | 0.76 | 0.78 |
| HSPB1_HUMAN | 0.3 | P04792 | HSPB1 | 0.09 | 0.04 | 0.23 | 0.01 | 0.12 | 0.25 |
| PDIA4_HUMAN | 0.3 | P13667 | PDIA4 | 0.04 | | | 0.01 | 0.05 | 0.21 |
| PTPRJ_HUMAN | 0.29 | Q12913 | PTPRJ | 0.5 | 0.29 | 0.64 | | 0.93 | 0.99 |
| CD177_HUMAN | 0.29 | Q8N6Q3 | CD177 | 0.68 | | 0.69 | | 0.75 | 0.8 |
| H2A1D_HUMAN | 0.29 | P20671 | HIST1H2AD | | | | | | |
| NNTM_HUMAN | 0.29 | Q13423 | NNT | | | 0.05 | 0.02 | 0.29 | 0.29 |
| IBP3_HUMAN | 0.29 | P17936 | IGFBP3 | 0.78 | 0.5 | 0.09 | 0.02 | 0.87 | 0.87 |
| STK24_HUMAN | 0.29 | Q9Y6E0 | STK24 | 0 | | 0.63 | | 0.8 | 0.82 |
| 1433G_HUMAN | 0.28 | P61981 | YWHAG | 0.76 | | 0.05 | | 0.82 | 0.83 |
| ITA2_HUMAN | 0.28 | P17301 | ITGA2 | 0.74 | 0.67 | 0.63 | | 0.85 | 0.86 |
| TCPD_HUMAN | 0.28 | P50991 | CCT4 | 0.69 | 0.02 | 0.03 | | 0.8 | 0.8 |
| GNA13_HUMAN | 0.28 | Q14344 | GNA13 | 0.01 | 0.07 | 0.08 | | 0.67 | 0.68 |
| AT5F1_HUMAN | 0.28 | P24539 | ATP5F1 | | | | | | |
| SODE_HUMAN | 0.28 | P08294 | SOD3 | 0.04 | 0.06 | 0.04 | | 0.09 | 0.17 |
| LDHA_HUMAN | 0.28 | P00338 | LDHA | 0.16 | 0.27 | 0.28 | 0.04 | 0.35 | 0.36 |
| PROF1_HUMAN | 0.28 | P07737 | PFN1 | 0.02 | 0.06 | 0.02 | | 0.77 | 0.77 |
| FHR4_HUMAN | 0.28 | Q92496 | CFHR4 | | | | | | 0.23 |
| HV301_HUMAN | 0.28 | | | | | | | | |
| PDE5A_HUMAN | 0.27 | O76074 | PDE5A | 0.26 | 0.23 | 0.26 | 0.21 | 1 | 1 |
| MUCB_HUMAN | 0.27 | | | | | | | | |
| IF5A1_HUMAN | 0.27 | P63241 | EIF5A | 0.16 | 0.04 | 0.11 | 0.04 | 0.17 | 0.19 |
| GP126_HUMAN | 0.27 | | | | | | | | |
| EF1A1_HUMAN | 0.27 | P68104 | EEF1A1 | 0.42 | 0.01 | 0.46 | | 0.76 | 0.79 |
| TFR1_HUMAN | 0.27 | P02786 | TFRC | 0.6 | 0.29 | 0.19 | 0.02 | 0.65 | 0.66 |
| ZA2G_HUMAN | 0.27 | P25311 | AZGP1 | 0.05 | 0.05 | 0.02 | | 0.27 | 0.29 |
| LOX12_HUMAN | 0.27 | P18054 | ALOX12 | 0.01 | | | | 0.68 | 0.7 |
| SNP23_HUMAN | 0.27 | O00161 | SNAP23 | 0.17 | | 0.01 | 0.02 | 0.19 | 0.2 |
| ATPG_HUMAN | 0.27 | P36542 | ATP5C1 | | | | | | |
| DHB4_HUMAN | 0.27 | P51659 | HSD17B4 | 0.01 | | 0.5 | | 0.08 | 0.52 |
| PNPH_HUMAN | 0.27 | P00491 | PNP | 0.05 | 0.05 | 0.04 | | 0.51 | 0.57 |
| RAB1B_HUMAN | 0.27 | Q9H0U4 | RAB1B | | | | | 0.19 | 0.19 |
| HGFA_HUMAN | 0.26 | Q04756 | HGFAC | 0.63 | 0.05 | 0.51 | 0.13 | 0.77 | 0.79 |
| H2AJ_HUMAN | 0.26 | Q9BTM1 | H2AFJ | | | 0.25 | | 0.76 | 0.77 |
| PIP_HUMAN | 0.26 | P12273 | PIP | 0.06 | 0.01 | 0.04 | | 0.24 | 0.24 |
| PSB6_HUMAN | 0.26 | P28072 | PSMB6 | 0.85 | 0.27 | 0.79 | 1 | 1 | 1 |
| TCPQ_HUMAN | 0.26 | P50990 | CCT8 | | | 0.07 | | 0.79 | 0.79 |
| TBA1C_HUMAN | 0.26 | Q9BQE3 | TUBA1C | 1 | 1 | 1 | 1 | 1 | 1 |
| 1A24_HUMAN | 0.26 | P05534 | HLA-A | 0.87 | | 0.58 | | 1 | 1 |
| DEST_HUMAN | 0.25 | P60981 | DSTN | | 0.05 | | | 0.06 | 0.07 |
| TETN_HUMAN | 0.25 | P05452 | CLEC3B | 0.02 | 0.02 | 0.02 | 0.03 | 0.22 | 0.22 |
| ARPC4_HUMAN | 0.25 | P59998 | ARPC4 | | 0.02 | | | 0.02 | 0.02 |
| MIF_HUMAN | 0.25 | P14174 | MIF | 0.06 | 0.03 | 0.05 | | 0.06 | 0.06 |
| SEPP1_HUMAN | 0.25 | P49908 | SELENOP | 0.01 | 0.03 | | | 0.08 | 0.08 |
| SCG1_HUMAN | 0.25 | P05060 | CHGB | 0.02 | 0.07 | 0.05 | | 0.08 | 0.08 |
| TCPZ_HUMAN | 0.25 | P40227 | CCT6A | 0.69 | | 0.69 | | 0.81 | 0.81 |
| ACON_HUMAN | 0.25 | Q99798 | ACO2 | | | | | 0.27 | 0.3 |
| PI16_HUMAN | 0.25 | Q6UXB8 | PI16 | | | 0.01 | | 0.02 | 0.03 |
| DPYL2_HUMAN | 0.25 | Q16555 | DPYSL2 | 0.05 | | 0.07 | | 0.49 | 0.49 |
| DHRS7_HUMAN | 0.25 | Q9Y394 | DHRS7 | | | | | 0.05 | 0.06 |
| KRT36_HUMAN | 0.25 | O76013 | KRT36 | | | 0.53 | | 0.7 | 0.74 |
| DHSA_HUMAN | 0.24 | | | | | | | | |
| KPRP_HUMAN | 0.24 | Q5T749 | KPRP | | 0.01 | | | 0.01 | 0.01 |
| FA7_HUMAN | 0.24 | P08709 | F7 | 0.64 | 0.02 | 0.03 | 0.01 | 0.68 | 0.68 |
| DBNL_HUMAN | 0.24 | Q9UJU6 | DBNL | 0.15 | | | | 0.76 | 0.78 |
| RL40_HUMAN | 0.23 | P62987 | UBA52 | 0.01 | | 0.82 | | 0.82 | 1 |
| DERM_HUMAN | 0.23 | Q07507 | DPT | 0.03 | 0 | 0.03 | | 0.23 | 0.23 |
| RELN_HUMAN | 0.23 | P78509 | RELN | 0.64 | 0.06 | 0.08 | 0.24 | 0.75 | 0.79 |
| PGRC1_HUMAN | 0.23 | O00264 | PGRMC1 | 0.05 | 0.03 | 0.05 | 0.02 | 0.17 | 0.18 |
| PZP_HUMAN | 0.23 | P20742 | PZP | | | | 0.02 | 0.06 | 0.06 |
| MYH4_HUMAN | 0.23 | Q9Y623 | MYH4 | 0.17 | | | | 0.17 | 0.17 |
| ANXA3_HUMAN | 0.23 | P12429 | ANXA3 | 0.04 | 0.01 | 0 | | 0.29 | 0.29 |
| GGT1_HUMAN | 0.23 | P19440 | GGT1 | 0.02 | 0.02 | 0.22 | | 0.21 | 0.28 |
| PECA1_HUMAN | 0.23 | P16284 | PECAM1 | 0.09 | 0.03 | 0.06 | 0.01 | 0.23 | 0.23 |
| TGFI1_HUMAN | 0.23 | O43294 | TGFB1I1 | | | 0.05 | | 0.24 | 0.25 |
| RAB10_HUMAN | 0.23 | P61026 | RAB10 | 0.17 | | | | 0.24 | 0.24 |
| GLU2B_HUMAN | 0.22 | P14314 | PRKCSH | 0.09 | | | | 0.09 | 0.09 |
| CDHR2_HUMAN | 0.22 | Q9BYE9 | CDHR2 | | | | | 0.03 | 0.04 |
| PEAR1_HUMAN | 0.22 | Q5VY43 | PEAR1 | 0 | | | | 0 | 0.02 |
| BTK_HUMAN | 0.22 | Q06187 | BTK | 1 | 0.24 | 0.86 | 0.31 | 1 | 1 |
| RB11A_HUMAN | 0.22 | P62491 | RAB11A | 0.17 | 0.01 | | | 0.19 | 0.19 |
| EHD3_HUMAN | 0.22 | Q9NZN3 | EHD3 | | | 0.3 | | 0.66 | 0.68 |
| RHOA_HUMAN | 0.22 | P61586 | RHOA | 0.92 | 0.05 | 0.04 | | 1 | 1 |
| ECHB_HUMAN | 0.22 | P55084 | HADHB | 0.01 | | | | 0.5 | 0.5 |
| CALM_HUMAN | 0.22 | | | | | | | | |
| GAPR1_HUMAN | 0.21 | Q9H4G4 | GLIPR2 | 0 | | 0.01 | | 0.01 | 0.02 |
| K2C73_HUMAN | 0.21 | Q86Y46 | KRT73 | | | 0.1 | | 0.74 | 0.74 |
| VAMP8_HUMAN | 0.21 | Q9BV40 | VAMP8 | 0.04 | | 0.09 | | 0.8 | 0.81 |
| CHCH3_HUMAN | 0.21 | | | | | | | | |
| CISY_HUMAN | 0.21 | O75390 | CS | | | | | | |
| KAP0_HUMAN | 0.21 | P10644 | PRKAR1A | 0.98 | 0.56 | 0.72 | | 1 | 1 |
| RAB7A_HUMAN | 0.2 | P51149 | RAB7A | | | | | 0.21 | 0.21 |
| COR1A_HUMAN | 0.2 | P31146 | CORO1A | | | | | 0.2 | 0.2 |
| KV110_HUMAN | 0.2 | | | | | | | | |
| PCSK9_HUMAN | 0.2 | Q8NBP7 | PCSK9 | 0.08 | | 0.04 | | 0.69 | 0.69 |
| MENT_HUMAN | 0.2 | Q9BUN1 | MENT | | | | | | |
| ESYT1_HUMAN | 0.2 | Q9BSJ8 | ESYT1 | 0.04 | | | | 0.06 | 0.06 |
| ETFA_HUMAN | 0.2 | P13804 | ETFA | | | 0.65 | | | 0.78 |
| ANX11_HUMAN | 0.2 | P50995 | ANXA11 | 0.02 | | | | 0.11 | 0.11 |
| ARPC2_HUMAN | 0.2 | O15144 | ARPC2 | 0.02 | | 0.63 | | 0.57 | 0.75 |
| LECT2_HUMAN | 0.2 | O14960 | LECT2 | 0.03 | | | | 0.23 | 0.23 |
| TCPB_HUMAN | 0.19 | P78371 | CCT2 | 0.71 | | 0.68 | | 0.81 | 0.81 |
| RAC1_HUMAN | 0.19 | P63000 | RAC1 | 0.75 | 0.04 | 0.57 | | 1 | 1 |
| SRGN_HUMAN | 0.19 | P10124 | SRGN | 0.22 | | 0.23 | 0.22 | 0.72 | 0.72 |
| RAB21_HUMAN | 0.19 | Q9UL25 | RAB21 | | | 0.02 | | 0.07 | 0.07 |
| ADDB_HUMAN | 0.19 | P35612 | ADD2 | 0 | | 0 | | | 0.01 |
| AT1A1_HUMAN | 0.19 | P05023 | ATP1A1 | 0.86 | | 0.8 | | 1 | 1 |
| B4GT1_HUMAN | 0.19 | P15291 | B4GALT1 | 0.59 | | 0 | | 0.78 | 0.8 |
| CCL5_HUMAN | 0.19 | P13501 | CCL5 | 0.06 | 0.05 | 0.18 | 0.01 | 0.36 | 0.38 |
| RAB5C_HUMAN | 0.19 | P51148 | RAB5C | | | | | 0.03 | 0.07 |
| PLEC_HUMAN | 0.19 | Q15149 | PLEC | 0 | 0.06 | 0.75 | 0.02 | 0.81 | 0.82 |
| K1H1_HUMAN | 0.19 | Q15323 | KRT31 | | | 0.52 | | 0.74 | 0.75 |
| ARPC5_HUMAN | 0.19 | O15511 | ARPC5 | | | | | 0.04 | 0.04 |
| 1B15_HUMAN | 0.19 | P30464 | HLA-B | 0.79 | | | | 0.89 | 0.97 |
| DLDH_HUMAN | 0.19 | P09622 | DLD | | | | | 0.02 | 0.63 |
| CSRP1_HUMAN | 0.19 | P21291 | CSRP1 | 0.02 | | 0 | | 0.04 | 0.04 |
| PSA1_HUMAN | 0.19 | P25786 | PSMA1 | 0.33 | 0.28 | 0.79 | 1 | 1 | 1 |
| LV403_HUMAN | 0.18 | A0A075B6K6 | IGLV4-3 | | | | | | |
| CKLF5_HUMAN | 0.18 | Q96DZ9 | CMTM5 | | 0.05 | 0 | | 0.19 | 0.2 |
| STIM1_HUMAN | 0.18 | Q13586 | STIM1 | 0.1 | 0.67 | 0.44 | 0.1 | 0.81 | 0.82 |
| DIAP1_HUMAN | 0.18 | O60610 | DIAPH1 | 0.63 | 0.5 | 0.1 | | 0.73 | 0.74 |
| ARC1B_HUMAN | 0.18 | O15143 | ARPC1B | 0.01 | | 0.1 | | 0.53 | 0.54 |
| FCN2_HUMAN | 0.18 | Q15485 | FCN2 | 0.18 | | | | 0.23 | 0.23 |
| EF1D_HUMAN | 0.18 | P29692 | EEF1D | 0.04 | | 0.06 | | 0.14 | 0.14 |
| SUCB1_HUMAN | 0.18 | Q9P2R7 | SUCLA2 | | | | | 0.02 | 0.19 |
| FLOT1_HUMAN | 0.17 | O75955 | FLOT1 | 0.05 | | | | 0.54 | 0.54 |
| PRG3_HUMAN | 0.17 | Q9Y2Y8 | PRG3 | | | | | | |
| FAT2_HUMAN | 0.17 | Q9NYQ8 | FAT2 | 0.04 | 0.02 | 0 | | 0.15 | 0. 5 |
| LYN_HUMAN | 0.17 | P07948 | LYN | 0.76 | 0.3 | 0.85 | 0.27 | 1 | 1 |
| CADH1_HUMAN | 0.17 | P12830 | CDH1 | 0.97 | 0.69 | 0.95 | 0.66 | 1 | 1 |
| TFPI1_HUMAN | 0.17 | P10646 | TFPI | 0.58 | 0.07 | 0.07 | 0.05 | 0.67 | 0.67 |
| H33_HUMAN | 0.17 | P84243 | H3F3A | 0.69 | | 1 | | 1 | 1 |
| H33_HUMAN | 0.17 | P84243 | H3F3B | 0.74 | 0.56 | 1 | | 0.94 | 1 |
| FBLN5_HUMAN | 0.17 | Q9UBX5 | FBLN5 | 0.56 | 0.04 | 0.07 | | 0.71 | 0.71 |
| HV304_HUMAN | 0.17 | | | | | | | | |
| ABHGA_HUMAN | 0.17 | O95870 | ABHD16A | | | | | | 0.01 |
| MET7A_HUMAN | 0.17 | Q9H8H3 | METTL7A | 0 | | 0 | | 0.03 | 0.21 |
| PCD18_HUMAN | 0.17 | Q9HCL0 | PCDH18 | | | 0 | | 0.2 | 0.21 |
| MLEC_HUMAN | 0.17 | Q14165 | MLEC | | | | | 0.03 | 0.03 |
| BPIB1_HUMAN | 0.16 | Q8TDL5 | BPIFB1 | 0.57 | | 0.41 | | 0.64 | 0.68 |
| LV106_HUMAN | 0.16 | | | | | | | | |
| DYH7_HUMAN | 0.16 | Q8WXX0 | DNAH7 | | | | | | |
| THIO_HUMAN | 0.16 | P10599 | TXN | 0.1 | 0.05 | 0.06 | 0.05 | 0.8 | 0.82 |
| ILF2_HUMAN | 0.16 | Q12905 | ILF2 | 0.08 | 0.16 | 0.08 | 0.3 | 0.25 | 0.35 |
| RALB_HUMAN | 0.16 | P11234 | RALB | 0.51 | | | | 0.69 | 0.74 |
| KV119_HUMAN | 0.16 | | | | | | | | |
| TITIN_HUMAN | 0.16 | Q8WZ42 | TTN | 0.5 | 0.28 | 0.43 | 0.03 | 0.79 | 0.8 |
| BASP1_HUMAN | 0.16 | P80723 | BASP1 | 0.01 | | 0.01 | | 0.25 | 0.27 |
| EF1A3_HUMAN | 0.16 | Q5VTE0 | EEF1A1P5 | | | | | | |
| RASA3_HUMAN | 0.16 | Q14644 | RASA3 | 0.73 | | 0.08 | | 0.8 | 0.81 |
| S10AE_HUMAN | 0.15 | Q9HCY8 | S100A14 | 0.08 | 0 | | | 0.25 | 0.26 |
| BTD_HUMAN | 0.15 | P43251 | BTD | | | | 0.02 | 0.15 | 0.24 |
| RPN2_HUMAN | 0.15 | P04844 | RPN2 | 0.19 | 0.02 | | | 0.24 | 0.29 |
| BLVRB_HUMAN | 0.15 | P30043 | BLVRB | | | 0.02 | | 0.12 | 0.12 |
| PKP1_HUMAN | 0.15 | Q13835 | PKP1 | 0.02 | | | | 0.8 | 0.8 |
| UN13D_HUMAN | 0.15 | Q70J99 | UNC13D | | | | | | 0.03 |
| ILEU_HUMAN | 0.15 | P30740 | SERPINB1 | | 0.01 | 0.06 | | 0.12 | 0.12 |
| K2C3_HUMAN | 0.15 | P12035 | KRT3 | 0.02 | | 0.1 | | 0.66 | 0.67 |
| ATPO_HUMAN | 0.15 | P48047 | ATP5O | | | | | | |
| POSTN_HUMAN | 0.15 | Q15063 | POSTN | 0.28 | 0.23 | 0.25 | 0.12 | 0.32 | 0.32 |
| MAP1A_HUMAN | 0.15 | P78559 | MAP1A | | 0.03 | 0.05 | | 0.05 | 0.06 |
| MGP_HUMAN | 0.15 | P08493 | MGP | 0.03 | | 0.19 | | 0.23 | 0.26 |
| KI20B_HUMAN | 0.15 | Q96Q89 | KIF20B | | 0.01 | 0.57 | | 0.56 | 0.68 |
| COX41_HUMAN | 0.15 | P13073 | COX4I1 | 0.63 | | 0.16 | | 0.71 | 0.74 |
| GPX1_HUMAN | 0.15 | P07203 | GPX1 | 0.07 | | 0.09 | 0.01 | 0.62 | 0.62 |
| FHR3_HUMAN | 0.15 | Q02985 | CFHR3 | | | | | 0.01 | 0.02 |
| GSTK1_HUMAN | 0.14 | Q9Y2Q3 | GSTK1 | | | 0.22 | | 0.02 | 0.22 |
| QCR1_HUMAN | 0.14 | P31930 | UQCRC1 | | | | | 0.03 | 0.06 |
| PARVB_HUMAN | 0.14 | Q9HBI1 | PARVB | | 0.03 | | | 0.06 | 0.06 |
| CX6B1_HUMAN | 0.14 | P14854 | COX6B1 | 0.63 | | | | 0.66 | 0.66 |
| H32_HUMAN | 0.14 | Q71DI3 | HIST2H3A | | | | | | |
| H32_HUMAN | 0.14 | Q71DI3 | HIST2H3C | | | | | | |
| H32_HUMAN | 0.14 | Q71DI3 | HIST2H3D | | | | | | |
| MGAT1_HUMAN | 0.14 | P26572 | MGAT1 | | | 0.04 | | 0.03 | 0.05 |
| K2C80_HUMAN | 0.14 | Q6KB66 | KRT80 | | | | | 0.63 | 0.63 |
| RB11B_HUMAN | 0.14 | Q15907 | RAB11B | | | | | | |
| G6B_HUMAN | 0.14 | O95866 | MPIG6B | | | | | | |
| MARE2_HUMAN | 0.14 | Q15555 | MAPRE2 | 0.01 | 0.05 | | | 0.06 | 0.07 |
| FYB_HUMAN | 0.14 | | | | | | | | |
| SPB12_HUMAN | 0.14 | Q96P63 | SERPINB12 | | | | | 0.05 | 0.06 |
| BASI_HUMAN | 0.13 | P35613 | BSG | 0.23 | 0.08 | 0.3 | 0.04 | 0.76 | 0.76 |
| HV103_HUMAN | 0.13 | A0A0C4DH29 | IGHV1-3 | | | | | | 0.01 |
| COX5A_HUMAN | 0.13 | P20674 | COX5A | | | | | 0.68 | 0.68 |
| ACTG_HUMAN | 0.13 | P63261 | ACTG1 | 0.42 | | 0.01 | | 0.77 | 1 |
| ESAM_HUMAN | 0.13 | Q96AP7 | ESAM | 0.02 | | 0.07 | | 0.52 | 0.53 |
| ARPC3_HUMAN | 0.13 | O15145 | ARPC3 | | | | | | 0.02 |
| WDR44_HUMAN | 0.13 | Q5JSH3 | WDR44 | | | | | | |
| ARGI1_HUMAN | 0.13 | P05089 | ARG1 | 0.58 | 0.04 | 0.04 | 0 | 0.62 | 0.62 |
| RNAS4_HUMAN | 0.13 | P34096 | RNASE4 | | | | | 0.03 | 0.03 |
| K2C1B_HUMAN | 0.13 | Q7Z794 | KRT77 | | | | | 0.71 | 0.71 |
| CAZA2_HUMAN | 0.13 | P47755 | CAPZA2 | | | | | 0.48 | 0.49 |
| HGFL_HUMAN | 0.13 | P26927 | MST1 | 0.03 | 0.04 | 0.01 | | 0.05 | 0.05 |
| CFAD_HUMAN | 0.13 | P00746 | CFD | 0.04 | | 0.02 | | 0.05 | 0.05 |
| ZG16B_HUMAN | 0.13 | Q96DA0 | ZG16B | 0.01 | 0 | | | 0.02 | 0.02 |
| CDC42_HUMAN | 0.13 | P60953 | CDC42 | 0.52 | 0.04 | 0.21 | | 0.78 | 0.82 |
| ODO2_HUMAN | 0.12 | P36957 | DLST | 0.02 | | | | 0.02 | 0.04 |
| AMPN_HUMAN | 0.12 | P15144 | ANPEP | 0.27 | 0.19 | 0.05 | 0.04 | 0.65 | 0.67 |
| SPR2E_HUMAN | 0.12 | P22531 | SPRR2E | | | | | 0.67 | 0.67 |
| SPTB2_HUMAN | 0.12 | Q01082 | SPTBN1 | 0 | 0.06 | 0.8 | | 0.84 | 0.84 |
| TMX1_HUMAN | 0.12 | Q9H3N1 | TMX1 | 0.01 | 0.04 | 0.03 | | 0.11 | 0.17 |
| CD44_HUMAN | 0.12 | P16070 | CD44 | 0.18 | 0.24 | 0.58 | 0.1 | 0.81 | 0.83 |
| CYFP1_HUMAN | 0.12 | Q7L576 | CYFIP1 | | 0.63 | | | 0.74 | 0.75 |
| RAB6B_HUMAN | 0.12 | Q9NRW1 | RAB6B | | | 0 | | | 0 |
| 1433B_HUMAN | 0.12 | P31946 | YWHAB | 0.69 | | | | 0.82 | 1 |
| HV107_HUMAN | 0.12 | | | | | | | | |
| 1B40_HUMAN | 0.12 | Q04826 | HLA-B | 0.79 | | | | 0.89 | 0.97 |
| COIA1_HUMAN | 0.11 | P39060 | COL18A1 | 0.67 | | 0.05 | | 0.79 | 0.79 |
| CATG_HUMAN | 0.11 | P08311 | CTSG | 0.65 | 0.01 | 0.45 | | 0.79 | 0.87 |
| VNN1_HUMAN | 0.11 | O95497 | VNN1 | | 0.04 | | | 0.05 | 0.07 |
| TIMP3_HUMAN | 0.11 | P35625 | TIMP3 | 0.15 | 0.06 | 0.14 | 0.02 | 0.71 | 0.72 |
| LY66F_HUMAN | 0.11 | Q5SQ64 | LY6G6F | | | 0.12 | | 0.66 | 0.66 |
| VIME_HUMAN | 0.11 | P08670 | VIM | 0.54 | 0.67 | 0.74 | 0.08 | 0.88 | 0.89 |
| K2C78_HUMAN | 0.11 | Q8N1N4 | KRT78 | | | | | 0.8 | 0.8 |
| LRC59_HUMAN | 0.11 | Q96AG4 | LRRC59 | | | | | 0.06 | 0.06 |
| 1B55_HUMAN | 0.11 | P30493 | HLA-B | 0.79 | | | | 0.89 | 0.97 |
| GBG5_HUMAN | 0.11 | P63218 | GNG5 | | | 0.75 | | 0.72 | 0.78 |
| DAAM1_HUMAN | 0.11 | Q9Y4D1 | DAAM1 | 0 | | 0.03 | | 0.5 | 0.51 |
| PTMA_HUMAN | 0.11 | P06454 | PTMA | | | 0.06 | 0.04 | 0.23 | 0.25 |
| PRDX1_HUMAN | 0.11 | Q06830 | PRDX1 | 0.65 | 0.04 | 0.07 | | 0.81 | 0.81 |
| DNM1L_HUMAN | 0.11 | O00429 | DNM1L | 0.04 | | 0.02 | | 0.79 | 0.8 |
| TALDO_HUMAN | 0.11 | P37837 | TALDO1 | 0.01 | | 0.05 | 0.02 | 0.09 | 0.09 |
| CASPE_HUMAN | 0.11 | P31944 | CASP14 | 0.09 | | 0.64 | | 0.75 | 0.78 |
| 1A03_HUMAN | 0.1 | P04439 | HLA-A | 0.87 | | 0.58 | | 1 | 1 |
| KV309_HUMAN | 0.1 | | | | | | | | |
| CRNN_HUMAN | 0.1 | Q9UBG3 | CRNN | 0.03 | 0.01 | 0.03 | | 0.07 | 0.07 |
| EM55_HUMAN | 0.1 | Q00013 | MPP1 | 0 | | | | | 0 |
| CD226_HUMAN | 0.1 | Q15762 | CD226 | 0.07 | 0.03 | 0.02 | 0.09 | 0.11 | 0.11 |
| S10A6_HUMAN | 0.1 | P06703 | S100A6 | 0.1 | 0.19 | 0.09 | 0.04 | 0.23 | 0.24 |
| NHRF1_HUMAN | 0.1 | O14745 | SLC9A3R1 | 0.06 | 0.21 | 0.25 | | 0.29 | 0.3 |
| FHOD1_HUMAN | 0.1 | Q9Y613 | FHOD1 | | | 0.01 | | 0.07 | 0.09 |
| 1B78_HUMAN | 0.1 | P30498 | HLA-B | 0.79 | | | | 0.89 | 0.97 |
| SPB3_HUMAN | 0.1 | P29508 | SERPINB3 | 0.07 | 0.09 | | | 0.28 | 0.28 |
| AIFM1_HUMAN | 0.1 | O95831 | AIFM1 | 0.05 | 0.2 | 0.07 | 0.03 | 0.27 | 0.28 |
| SPR1A_HUMAN | 0.1 | P35321 | SPRR1A | 0.02 | 0 | 0.63 | | 0.73 | 0.78 |
| NB5R3_HUMAN | 0.1 | P00387 | CYB5R3 | 0.03 | | 0.02 | 0.05 | 0.11 | 0.11 |
| PPM1A_HUMAN | 0.1 | P35813 | PPM1A | | 0 | | | 0.81 | 0.81 |
| TGM3_HUMAN | 0.1 | Q08188 | TGM3 | | | 0.01 | 0.01 | 0.92 | 0.92 |
| TPM1_HUMAN | 0.1 | P09493 | TPM1 | 0.5 | | 0.05 | | 0.76 | 0.76 |
| PRDX3_HUMAN | 0.1 | P30048 | PRDX3 | 0.06 | | 0.07 | | 0.13 | 0.13 |
| SPR2A_HUMAN | 0.1 | P35326 | SPRR2A | 0.03 | | | | 0.64 | 0.64 |
| XP32_HUMAN | 0.1 | Q5T750 | XP32 | | | | | | |
| NRP1_HUMAN | 0.1 | O14786 | NRP1 | 0.69 | 0.05 | 0.62 | | 0.78 | 0.82 |
| GBB1_HUMAN | 0.1 | P62873 | GNB1 | 0.66 | | 0.75 | | 0.81 | 1 |
| HCDH_HUMAN | 0.1 | Q16836 | HADH | | | 0.01 | | 0.02 | 0.02 |
| NDKB_HUMAN | 0.09 | P22392 | NME2 | 0.14 | | 0.02 | | 0.23 | 0.23 |
| SAR1A_HUMAN | 0.09 | Q9NR31 | SAR1A | | | | | | |
| HBG1_HUMAN | 0.09 | P69891 | HBG1 | | | | | 0.01 | 0.01 |
| SLAF5_HUMAN | 0.09 | Q9UIB8 | CD84 | | | 0 | | 0.51 | 0.55 |
| GANAB_HUMAN | 0.09 | Q14697 | GANAB | | | | | 0.01 | 0.04 |
| CBG_HUMAN | 0.09 | P08185 | SERPINA6 | 0.03 | | 0.04 | 0.03 | 0.09 | 0.09 |
| CH10_HUMAN | 0.09 | P61604 | HSPE1 | 0.02 | | | | 0.07 | 0.07 |
| TPP1_HUMAN | 0.09 | O14773 | TPP1 | | | 0.01 | | 0.02 | 0.03 |
| PCOC1_HUMAN | 0.09 | Q15113 | PCOLCE | 0 | 0 | | | 0.67 | 0.67 |
| UGGG1_HUMAN | 0.09 | Q9NYU2 | UGGT1 | | | | | | |
| IBP1_HUMAN | 0.09 | P08833 | IGFBP1 | 0.12 | 0.07 | 0.05 | 0.05 | 0.67 | 0.68 |
| RARR2_HUMAN | 0.09 | Q99969 | RARRES2 | 0.18 | | 0.05 | | 0.23 | 0.24 |
| RAB1A_HUMAN | 0.09 | P62820 | RAB1A | | | 0.04 | | 0.08 | 0.08 |
| ATRN_HUMAN | 0.09 | O75882 | ATRN | | | 0.07 | | 0.02 | 0.07 |
| GRAN_HUMAN | 0.09 | P28676 | GCA | | | 0 | | 0 | 0 |
| HINT2_HUMAN | 0.09 | Q9BX68 | HINT2 | 0.07 | 0.06 | | | 0.09 | 0.1 |
| LAC7_HUMAN | 0.09 | | | | | | | | |
| LASP1_HUMAN | 0.09 | Q14847 | LASP1 | 0.81 | 0.03 | 0.05 | | 0.97 | 0.97 |
| SEPT5_HUMAN | 0.09 | Q99719 | SEPT5 | 0.4 | 0.02 | 0.28 | | 0.45 | 0.46 |
| PP1B_HUMAN | 0.09 | P62140 | PPP1CB | | 0 | 0.63 | | 0.01 | 0.65 |
| CLC11_HUMAN | 0.09 | Q9Y240 | CLEC11A | 0.04 | 0.01 | | 0.01 | 0.05 | 0.06 |
| CYC_HUMAN | 0.09 | P99999 | CYCS | 0.09 | 0.09 | 0.76 | 0.05 | 0.78 | 0.85 |
| HV208_HUMAN | 0.09 | | | | | | | | |
| ANGI_HUMAN | 0.09 | P03950 | ANG | 0.12 | 0.07 | 0.14 | 0.03 | 0.2 | 0.27 |
| PDC6I_HUMAN | 0.09 | Q8WUM4 | PDCD6IP | 0.06 | | 0.06 | | 0.43 | 0.44 |
| CBPM_HUMAN | 0.09 | P14384 | CPM | 0.05 | | 0.05 | 0.02 | 0.28 | 0.28 |
| HV308_HUMAN | 0.09 | | | | | | | | |
| SLPI_HUMAN | 0.09 | P03973 | SLPI | 0.06 | 0.06 | 0 | | 0.11 | 0.15 |
| EF1G_HUMAN | 0.09 | P26641 | EEF1G | 0.02 | 0.04 | | | 0.14 | 0.14 |
| OLFM4_HUMAN | 0.09 | Q6UX06 | OLFM4 | 0.07 | 0.05 | 0.01 | | 0.31 | 0.31 |
| IBP2_HUMAN | 0.08 | P18065 | IGFBP2 | 0.28 | 0.19 | 0.27 | 0.01 | 0.65 | 0.65 |
| GDIA_HUMAN | 0.08 | P31150 | GDI1 | | | | | | 0.01 |
| SSBP_HUMAN | 0.08 | Q04837 | SSBP1 | 0.06 | | | | 0.09 | 0.09 |
| 1B18_HUMAN | 0.08 | P30466 | HLA-B | 0.79 | | | | 0.89 | 0.97 |
| AT2C1_HUMAN | 0.08 | P98194 | ATP2C1 | | | 0.28 | | 0.24 | 0.36 |
| IQGA2_HUMAN | 0.08 | Q13576 | IQGAP2 | 0.05 | | 0.01 | | 0.68 | 0.69 |
| ATP5H_HUMAN | 0.08 | O75947 | ATP5H | | | | | | |
| CD99_HUMAN | 0.08 | P14209 | CD99 | 0.05 | 0.03 | 0.12 | 0.14 | 0.53 | 0.57 |
| KAD2_HUMAN | 0.08 | P54819 | AK2 | | | | | 0.06 | 0.15 |
| VAPB_HUMAN | 0.08 | O95292 | VAPB | | | | | | |
| ADA10_HUMAN | 0.08 | O14672 | ADAM10 | 0.55 | 0.09 | 0.59 | 0.06 | 0.88 | 1 |
| MA1A1_HUMAN | 0.08 | P33908 | MAN1A1 | 0 | | 0.01 | | 0.06 | 0.07 |
| ATIF1_HUMAN | 0.08 | Q9UII2 | ATPIF1 | | | | | | |
| VATA_HUMAN | 0.08 | P38606 | ATP6V1A | | 0 | 0.28 | | 0.53 | 0.59 |
| PDIA5_HUMAN | 0.08 | Q14554 | PDIA5 | | | 0 | | 0.04 | 0.05 |
| MFGM_HUMAN | 0.08 | Q08431 | MFGE8 | 0.18 | 0.02 | 0.05 | | 0.26 | 0.26 |
| IGHD_HUMAN | 0.08 | P01880 | IGHD | | | 0.02 | | | 0.05 |
| DHE3_HUMAN | 0.08 | P00367 | GLUD1 | 0.06 | 0.01 | 0.02 | | 0.07 | 0.16 |
| GSTP1_HUMAN | 0.08 | P09211 | GSTP1 | 0.24 | 0.04 | 0.09 | 0.06 | 0.57 | 0.58 |
| PG12B_HUMAN | 0.08 | Q9BX93 | PLA2G12B | | | | | | |
| CPNS1_HUMAN | 0.08 | P04632 | CAPNS1 | 0.01 | 0.01 | 0.63 | | 0.77 | 0.79 |
| PNKD_HUMAN | 0.08 | Q8N490 | PNKD | 0.01 | | 0.07 | | 0.23 | 0.24 |
| CD47_HUMAN | 0.08 | Q08722 | CD47 | 0.26 | 0.06 | 0.11 | 0.09 | 0.3 | 0.31 |
| ESYT2_HUMAN | 0.08 | A0FGR8 | ESYT2 | 0.01 | | 0.11 | | 0.01 | 0.02 |
| ARL8B_HUMAN | 0.08 | Q9NVJ2 | ARL8B | | | | | | 0.03 |
| APLP2_HUMAN | 0.08 | Q06481 | APLP2 | 0.01 | 0.34 | 0.12 | | 0.73 | 0.74 |
| PCCB_HUMAN | 0.08 | P05166 | PCCB | | | | | 0.21 | 0.21 |
| 2AAA_HUMAN | 0.08 | P30153 | PPP2R1A | 1 | 0.82 | 0.69 | | 1 | 1 |
| TPP2_HUMAN | 0.08 | P29144 | TPP2 | | | | | 0.51 | 0.51 |
| S10A4_HUMAN | 0.08 | P26447 | S100A4 | 0.23 | 0.24 | 0.17 | | 0.29 | 0.3 |
| PCYXL_HUMAN | 0.08 | Q8NBM8 | PCYOX1L | | | | | 0.65 | 0.65 |
| AATM_HUMAN | 0.08 | P00505 | GOT2 | 0.02 | 0.05 | 0.05 | 0.01 | 0.35 | 0.35 |
| SUSD1_HUMAN | 0.08 | Q6UWL2 | SUSD1 | | | | | | 0 |
| TCPA_HUMAN | 0.08 | P17987 | TCP1 | 0.68 | | | | 0.79 | 0.79 |
| HTRA1_HUMAN | 0.08 | Q92743 | HTRA1 | 0.01 | | 0.03 | | 0.64 | 0.64 |
| ES1_HUMAN | 0.08 | P30042 | C21orf33 | | | | | | |
| GDIR2_HUMAN | 0.08 | P52566 | ARHGDIB | 0.06 | | | | 0.09 | 0.11 |
| PGBM_HUMAN | 0.08 | P98160 | HSPG2 | 0.66 | 0.03 | 0.07 | | 0.86 | 0.86 |
| GBB4_HUMAN | 0.08 | Q9HAV0 | GNB4 | 0.63 | | 0.75 | | 0.79 | 0.81 |
| FCN1_HUMAN | 0.08 | O00602 | FCN1 | 0.01 | | | | 0.17 | 0.19 |
| TXTP_HUMAN | 0.08 | P53007 | SLC25A1 | 0.1 | 0.02 | 0.02 | 0.01 | 0.25 | 0.27 |
| PCCA_HUMAN | 0.08 | P05165 | PCCA | | | | | 0.22 | 0.22 |
| SNAA_HUMAN | 0.08 | P54920 | NAPA | | | | | 0.06 | 0.06 |
| LCN1_HUMAN | 0.08 | P31025 | LCN1 | 0.02 | | 0.01 | | 0.17 | 0.18 |
| TM63A_HUMAN | 0.08 | | | | | | | | |
| TRI58_HUMAN | 0.08 | Q8NG06 | TRIM58 | 0.07 | | | | 0.1 | 0.1 |
| HMHA1_HUMAN | 0.08 | Q92619 | ARHGAP45 | | | | | 0 | 0 |
| S10AB_HUMAN | 0.08 | P31949 | S100A11 | 0.23 | 0.08 | 0.04 | | 0.28 | 0.28 |
| RAB32_HUMAN | 0.08 | Q13637 | RAB32 | | | 0 | | 0.05 | 0.05 |
| SQRD_HUMAN | 0.08 | | | | | | | | |
| GPC5C_HUMAN | 0.08 | Q9NQ84 | GPRC5C | | | | | 0.01 | 0.02 |
| ACADV_HUMAN | 0.08 | P49748 | ACADVL | | | 0.02 | | 0.02 | 0.03 |
| TKT_HUMAN | 0.08 | P29401 | TKT | 0.04 | 0.04 | 0.04 | | 0.1 | 0.11 |
| TM109_HUMAN | 0.08 | Q9BVC6 | TMEM109 | | | 0.01 | | | 0.01 |
| TRY2_HUMAN | 0.08 | P07478 | PRSS2 | | 0.04 | | | 0.06 | 0.06 |
| 1B35_HUMAN | 0.08 | P30685 | HLA-B | 0.79 | | | | 0.89 | 0.97 |
| TBA1A_HUMAN | 0.08 | Q71U36 | TUBA1A | 1 | 1 | 1 | 1 | 1 | 1 |
| G6PD_HUMAN | 0.08 | P11413 | G6PD | 0.67 | | 0.73 | 0.02 | 0.82 | 0.85 |
| FLII_HUMAN | 0.07 | Q13045 | FLII | 0.02 | | | | 0.06 | 0.07 |
| BMP1_HUMAN | 0.07 | P13497 | BMP1 | 0.22 | | 0.11 | | 0.75 | 0.77 |
| TPSN_HUMAN | 0.07 | O15533 | TAPBP | 0.04 | 0.02 | 0.1 | 0.02 | 0.07 | 0.11 |
| PRDX5_HUMAN | 0.07 | P30044 | PRDX5 | 0.07 | 0.05 | 0.05 | 0.04 | 0.69 | 0.69 |
| ANGL3_HUMAN | 0.07 | Q9Y5C1 | ANGPTL3 | | | 0.04 | | 0.53 | 0.53 |
| KV308_HUMAN | 0.07 | | | | | | | | |
| ADDA_HUMAN | 0.07 | P35611 | ADD1 | 0.04 | | 0.63 | | 0.65 | 0.74 |
| ROA2_HUMAN | 0.07 | P22626 | HNRNPA2B1 | 0.45 | 0.22 | 0.36 | | 0.52 | 0.52 |
| LCAT_HUMAN | 0.07 | P04180 | LCAT | | | 0.03 | | 0.1 | 0.1 |
| ECE1_HUMAN | 0.07 | P42892 | ECE1 | 0.02 | 0.02 | 0.07 | | 0.74 | 0.75 |
| IGF2_HUMAN | 0.07 | P01344 | IGF2 | 0.55 | 0.11 | 0.11 | 0.03 | 0.82 | 0.82 |
| GSHR_HUMAN | 0.07 | P00390 | GSR | 0.54 | | 1 | 1 | 1 | 1 |
| ANO6_HUMAN | 0.07 | Q4KMQ2 | ANO6 | | | 0 | | 0.26 | 0.3 |
| TSN9_HUMAN | 0.07 | O75954 | TSPAN9 | | | | | 0.01 | 0.28 |
| LMAN2_HUMAN | 0.07 | Q12907 | LMAN2 | 0.15 | | | | 0.3 | 0.3 |
| SC22B_HUMAN | 0.07 | O75396 | SEC22B | | | | | 0.01 | 0.01 |
| DYL1_HUMAN | 0.07 | P63167 | DYNLL1 | | | 0.75 | | 0.75 | 0.79 |
| G6PI_HUMAN | 0.07 | P06744 | GPI | 0.64 | 0.08 | 0.09 | 0.04 | 0.81 | 0.81 |
| PDLI7_HUMAN | 0.07 | Q9NR12 | PDLIM7 | 0.05 | 0.02 | 0.03 | 0.04 | 0.5 | 0.51 |
| FCGBP_HUMAN | 0.07 | Q9Y6R7 | FCGBP | 0.01 | | 0 | | 0.04 | 0.04 |
| RINI_HUMAN | 0.07 | P13489 | RNH1 | 0.03 | 0.02 | 0.02 | | 0.07 | 0.1 |
| IMMT_HUMAN | 0.07 | | | | | | | | |
| PIGR_HUMAN | 0.07 | P01833 | PIGR | 0.04 | 0.03 | 0.07 | 0.02 | 0.23 | 0.27 |
| P2RX1_HUMAN | 0.07 | P51575 | P2RX1 | 0.01 | 0 | 0.03 | 0.01 | 0.05 | 0.06 |
| GSTO1_HUMAN | 0.07 | P78417 | GSTO1 | 0.05 | 0.01 | 0.01 | | 0.08 | 0.17 |
| STX4_HUMAN | 0.07 | Q12846 | STX4 | | | | 0.02 | 0.71 | 0.72 |
| REEP5_HUMAN | 0.07 | Q00765 | REEP5 | 0.07 | | | | 0.52 | 0.52 |
| H15_HUMAN | 0.07 | P16401 | HIST1H1B | 0.72 | | 0.75 | | 0.81 | 0.82 |
| 6PGD_HUMAN | 0.07 | P52209 | PGD | 0.04 | 0.02 | 0.04 | | 0.08 | 0.1 |
| STX7_HUMAN | 0.07 | O15400 | STX7 | | 0 | | | 0.06 | 0.06 |
| NCAM1_HUMAN | 0.07 | P13591 | NCAM1 | 0.59 | 0.05 | 0.85 | 0.28 | 0.85 | 0.91 |
| K6PP_HUMAN | 0.07 | | | | | | | | |
| HS90B_HUMAN | 0.07 | P08238 | HSP90AB1 | 1 | 0.27 | 0.93 | 0.86 | 1 | 1 |
| PRAF3_HUMAN | 0.07 | O75915 | ARL6IP5 | 0.11 | 0.07 | 0.02 | | 0.25 | 0.25 |
| EMD_HUMAN | 0.07 | P50402 | EMD | 0.12 | 0.07 | 0.05 | 0.06 | 0.14 | 0.14 |
| SMIM1_HUMAN | 0.07 | B2RUZ4 | SMIM1 | | | | | | 0.02 |
| M2OM_HUMAN | 0.07 | Q02978 | SLC25A11 | | | | | 0.02 | 0.02 |
| CTGF_HUMAN | 0.07 | P29279 | CTGF | 0.26 | 0.15 | 0.28 | 0.03 | 0.33 | 0.34 |
| PPIB_HUMAN | 0.07 | P23284 | PPIB | 0.03 | 0.04 | 0.03 | | 0.58 | 0.59 |
| INF2_HUMAN | 0.07 | Q27J81 | INF2 | | | | | 0.11 | 0.11 |
| HV302_HUMAN | 0.07 | | | | | | | | |
| OSTP_HUMAN | 0.07 | P10451 | SPP1 | 0.3 | 0.11 | 0.29 | 0.14 | 0.72 | 0.72 |
| SH3L3_HUMAN | 0.07 | Q9H299 | SH3BGRL3 | | | 0.01 | | 0.06 | 0.06 |
| SKAP2_HUMAN | 0.07 | O75563 | SKAP2 | 0.24 | 0 | 0.36 | 0.02 | 0.24 | 0.42 |
| KRT35_HUMAN | 0.07 | Q92764 | KRT35 | | | 0.52 | | 0.74 | 0.75 |
| RTN3_HUMAN | 0.07 | O95197 | RTN3 | 0.5 | | 0.03 | | 0.54 | 0.55 |
| HV318_HUMAN | 0.07 | | | | | | | | |
| VNN2_HUMAN | 0.07 | O95498 | VNN2 | | | 0.04 | | | 0.04 |
| A16A1_HUMAN | 0.06 | Q8IZ83 | ALDH16A1 | | | | | 0.13 | 0.13 |
| ADT3_HUMAN | 0.06 | P12236 | SLC25A6 | 0.01 | | | | 0.72 | 0.72 |
| SHRM3_HUMAN | 0.06 | Q8TF72 | SHROOM3 | 0 | | | 0 | 0 | 0.02 |
| GRP2_HUMAN | 0.06 | Q7LDG7 | RASGRP2 | | | 0.5 | | 0.67 | 0.72 |
| ERP44_HUMAN | 0.06 | Q9BS26 | ERP44 | 0.03 | | | 0.02 | 0.03 | 0.05 |
| 1433T_HUMAN | 0.06 | P27348 | YWHAQ | 0.69 | | | | 0.76 | 0.76 |
| NPS3A_HUMAN | 0.06 | Q9UFN0 | NIPSNAP3A | | | 0 | | | 0 |
| CNST_HUMAN | 0.06 | Q6PJW8 | CNST | | | 0.03 | | | 0.08 |
| K1C18_HUMAN | 0.06 | P05783 | KRT18 | 0.16 | 0.07 | 0.51 | 0.04 | 0.3 | 0.61 |
| BAF_HUMAN | 0.06 | O75531 | BANF1 | 0.05 | | 0.02 | | 0.09 | 0.11 |
| MMP2_HUMAN | 0.06 | P08253 | MMP2 | 0.7 | 0.2 | 0.29 | 0.21 | 0.87 | 0.95 |
| CKAP5_HUMAN | 0.06 | Q14008 | CKAP5 | 0.06 | | 0.63 | 0.01 | 0.73 | 0.76 |
| KRT84_HUMAN | 0.06 | Q9NSB2 | KRT84 | | | 0.5 | | 0.68 | 0.71 |
| CASP3_HUMAN | 0.06 | P42574 | CASP3 | 0.63 | 0.09 | 0.11 | 0.06 | 0.87 | 0.87 |
| EHD1_HUMAN | 0.06 | Q9H4M9 | EHD1 | 0.27 | | | | 0.69 | 0.7 |
| ANXA5_HUMAN | 0.06 | P08758 | ANXA5 | 0.07 | 0.06 | 0.08 | 0.06 | 0.7 | 0.7 |
| HNRPU_HUMAN | 0.06 | Q00839 | HNRNPU | 0.04 | | 0.03 | | 0.05 | 0.05 |
| OST48_HUMAN | 0.06 | P39656 | DDOST | | | | | 0.13 | 0.13 |
| LRRF2_HUMAN | 0.06 | Q9Y608 | LRRFIP2 | | | | | | 0.02 |
| KRT83_HUMAN | 0.06 | P78385 | KRT83 | | | | | 0.63 | 0.63 |
| RHG01_HUMAN | 0.06 | Q07960 | ARHGAP1 | | | | | 0.16 | 0.16 |
| MCU_HUMAN | 0.06 | Q8NE86 | MCU | | 0.04 | | | 0.25 | 0.25 |
| FCERG_HUMAN | 0.06 | P30273 | FCER1G | | | 0.02 | | 0.05 | 0.2 |
| AL5AP_HUMAN | 0.06 | P20292 | ALOX5AP | 0 | | | | 0.19 | 0.19 |
| RETN_HUMAN | 0.06 | Q9HD89 | RETN | 0.07 | 0.03 | | 0.21 | 0.28 | 0.3 |
| SODC_HUMAN | 0.06 | P00441 | SOD1 | 0.04 | 0.02 | 0.05 | | 0.72 | 0.72 |
| LV001_HUMAN | 0.06 | | | | | | | | |
| CMIP_HUMAN | 0.06 | Q8IY22 | CMIP | | | 0.2 | 0.02 | 0.07 | 0.22 |
| TXND5_HUMAN | 0.06 | Q8NBS9 | TXNDC5 | 0.06 | 0.01 | | | 0.16 | 0.16 |
| RAN_HUMAN | 0.06 | P62826 | RAN | 0.01 | | 0.01 | | 0.76 | 0.76 |
| PLSL_HUMAN | 0.06 | P13796 | LCP1 | 0.49 | 0.02 | 0.34 | 0.03 | 0.58 | 0.58 |
| TSN14_HUMAN | 0.06 | Q8NG11 | TSPAN14 | 0.01 | | | | 0.01 | 0.02 |
| HYOU1_HUMAN | 0.06 | Q9Y4L1 | HYOU1 | 0.51 | 0.05 | 0.06 | | 0.67 | 0.69 |
| PTPRC_HUMAN | 0.06 | P08575 | PTPRC | 0.9 | 0.79 | 0.69 | 0.21 | 1 | 1 |
| LEG7_HUMAN | 0.06 | P47929 | LGALS7 | | | | | | |
| LEG7_HUMAN | 0.06 | P47929 | LGALS7B | 0.01 | 0.01 | | | 0.3 | 0.3 |
| POF1B_HUMAN | 0.06 | Q8WVV4 | POF1B | | 0 | | | 0.02 | 0.03 |
| LAMB1_HUMAN | 0.06 | P07942 | LAMB1 | 0.72 | 0.01 | 0.62 | 0.26 | 0.8 | 0.81 |
| K1H2_HUMAN | 0.06 | Q14532 | KRT32 | | | 0.5 | | 0.71 | 0.74 |
| MTPN_HUMAN | 0.06 | P58546 | MTPN | | | | | 0.01 | 0.01 |
| 1433S_HUMAN | 0.05 | P31947 | SFN | 0.75 | 0.01 | 0.79 | | 0.82 | 0.84 |
| RHCE_HUMAN | 0.05 | P18577 | RHCE | | | | | 0.05 | 0.05 |
| VAPA_HUMAN | 0.05 | Q9P0L0 | VAPA | 0 | | 0 | | 0.05 | 0.06 |
| LCE2B_HUMAN | 0.05 | O14633 | LCE2B | 0.08 | | 0.1 | | 0.71 | 0.71 |
| LMNA_HUMAN | 0.05 | P02545 | LMNA | 0.82 | 0.56 | 0.63 | | 1 | 1 |
| INVO_HUMAN | 0.05 | P07476 | IVL | 0.09 | | | | 0.77 | 0.77 |
| NGAL_HUMAN | 0.05 | P80188 | LCN2 | 0.6 | 0.22 | 0.19 | 0.05 | 0.79 | 0.79 |
| GLNA_HUMAN | 0.05 | P15104 | GLUL | 0.04 | 0.01 | 0.08 | 0.05 | 0.21 | 0.22 |
| SCOT1_HUMAN | 0.05 | P55809 | OXCT1 | | | 0.04 | | 0.05 | 0.07 |
| CX7A2_HUMAN | 0.05 | P14406 | COX7A2 | 0.01 | | 0.07 | | 0.04 | 0.08 |
| TMED9_HUMAN | 0.05 | Q9BVK6 | TMED9 | | | | | | |
| LCE1B_HUMAN | 0.05 | Q5T7P3 | LCE1B | 0.08 | | | | 0.69 | 0.69 |
| A2ML1_HUMAN | 0.05 | A8K2U0 | A2ML1 | | | | | 0 | 0 |
| S10A2_HUMAN | 0.05 | P29034 | S100A2 | 0.11 | 0.07 | 0.08 | | 0.15 | 0.16 |
| DMKN_HUMAN | 0.05 | Q6E0U4 | DMKN | | 0.18 | | | 0.18 | 0.18 |
| TMEDA_HUMAN | 0.05 | P49755 | TMED10 | 0.15 | | | | 0.17 | 0.18 |
| FLOT2_HUMAN | 0.05 | Q14254 | FLOT2 | 0.52 | 0.08 | 0.02 | 0.02 | 0.57 | 0.57 |
| KLK10_HUMAN | 0.05 | O43240 | KLK10 | 0.08 | 0.04 | 0.07 | | 0.15 | 0.15 |
| LRBA_HUMAN | 0.05 | P50851 | LRBA | 0.02 | | | | 0.05 | 0.07 |
| FCG3A_HUMAN | 0.05 | P08637 | FCGR3A | | | | 0.02 | 0.68 | 0.68 |
| RAP2B_HUMAN | 0.05 | P61225 | RAP2B | 0.06 | | | | 0.06 | 0.06 |
| TYPH_HUMAN | 0.05 | P19971 | TYMP | 0.07 | 0.07 | 0.08 | 0.04 | 0.58 | 0.66 |
| TMM40_HUMAN | 0.05 | Q8WWA1 | TMEM40 | | | | | 0.04 | 0.05 |
| PCD12_HUMAN | 0.05 | Q9NPG4 | PCDH12 | | | | | | |
| RABP2_HUMAN | 0.05 | P29373 | CRABP2 | 0.09 | 0.06 | 0.15 | | 0.23 | 0.24 |
| PSB1_HUMAN | 0.05 | P20618 | PSMB1 | 0.32 | 0.27 | 0.79 | 1 | 1 | 1 |
| SPB13_HUMAN | 0.05 | Q9UIV8 | SERPINB13 | 0.01 | | | | 0.05 | 0.05 |
| PTTG_HUMAN | 0.05 | P53801 | PTTG1IP | 0 | | 0.04 | | 0.21 | 0.22 |
| PPGB_HUMAN | 0.04 | P10619 | CTSA | | | | | 0.06 | 0.06 |
| PA2GA_HUMAN | 0.04 | P14555 | PLA2G2A | 0.56 | | 0 | | 0.69 | 0.84 |
| RS9_HUMAN | 0 | P46781 | RPS9 | 0.02 | 0.01 | 0.07 | | 0.03 | 0.23 |
| HV315_HUMAN | 0 | A0A0B4J1V0 | IGHV3-15 | | | | | | |
| COLA1_HUMAN | 0 | Q96P44 | COL21A1 | 0.22 | | | | 0.7 | 0.7 |
| CWC25_HUMAN | 0 | Q9NXE8 | CWC25 | | | | | 0 | 0 |
| LV104_HUMAN | 0 | | | | | | | | |
| SAHH_HUMAN | 0 | P23526 | AHCY | | 0.01 | 0.02 | | 0.05 | 0.05 |
| DUS3L_HUMAN | 0 | Q96G46 | DUS3L | | | | | | |
| S20A2_HUMAN | 0 | Q08357 | SLC20A2 | | | | | | 0.06 |
| RL12_HUMAN | 0 | P30050 | RPL12 | | | | | 0.51 | 0.51 |
| CTL1_HUMAN | 0 | Q8WWI5 | SLC44A1 | 0.04 | | 0.51 | | 0.56 | 0.66 |
| IQCAL_HUMAN | 0 | A6NCM1 | IQCA1L | | | | | | |
| RS13_HUMAN | 0 | P62277 | RPS13 | 0.02 | 0.59 | 0.02 | | 0.8 | 0.8 |
| 1A11_HUMAN | 0 | P13746 | HLA-A | 0.87 | | 0.58 | | 1 | 1 |
| RAP1A_HUMAN | 0 | P62834 | RAP1A | 0.78 | | | | 0.8 | 1 |
| KV311_HUMAN | 0 | P04433 | IGKV3-11 | | | | | | |
| HV307_HUMAN | 0 | P01780 | IGHV3-7 | | | | | | 0.04 |
| EZRI_HUMAN | 0 | P15311 | EZR | 0.83 | 0.58 | 0.79 | 0.02 | 1 | 1 |
| H2A1C_HUMAN | 0 | Q93077 | HIST1H2AC | 0.88 | 0 | 0.74 | | 0.95 | 0.95 |
| KV307_HUMAN | 0 | | | | | | | | |
| KV303_HUMAN | 0 | | | | | | | | |
| KV401_HUMAN | 0 | P06312 | IGKV4-1 | | | | | 0 | 0.02 |
| LAC3_HUMAN | 0 | | | | | | | | |
| LACRT_HUMAN | 0 | Q9GZZ8 | LACRT | | | | | | |
| PKN3_HUMAN | 0 | Q6P5Z2 | PKN3 | | 0.01 | | | 0.07 | 0.08 |
| 1B56_HUMAN | 0 | P30495 | HLA-B | 0.79 | | | | 0.89 | 0.97 |
| KV301_HUMAN | 0 | | | | | | | | |
| 1B07_HUMAN | 0 | P01889 | HLA-B | 0.79 | | | | 0.89 | 0.97 |
| KV117_HUMAN | 0 | P01599 | IGKV1-17 | | | | | | |
| KV106_HUMAN | 0 | A0A0C4DH72 | IGKV1-6 | | | | | | |
| HS71L_HUMAN | 0 | P34931 | HSPA1L | 0.02 | | 0.44 | | 0.5 | 0.6 |
| AT1A2_HUMAN | 0 | P50993 | ATP1A2 | 0.2 | | 0.42 | | 0.71 | 0.74 |
| TRY1_HUMAN | 0 | P07477 | PRSS1 | 0.42 | 0.09 | 0.05 | 0.02 | 0.55 | 0.55 |
| EF1A2_HUMAN | 0 | Q05639 | EEF1A2 | 0.05 | 0.22 | | 0.02 | 0.27 | 0.28 |
| HV102_HUMAN | 0 | P23083 | IGHV1-2 | | | | | | 0.04 |
| SRBS2_HUMAN | 0 | O94875 | SORBS2 | 0 | 0.04 | 0.01 | | 0.06 | 0.06 |
| KV102_HUMAN | 0 | | | | | | | | |
| NPNT_HUMAN | 0 | Q6UXI9 | NPNT | 0 | | 0.03 | | 0.05 | 0.05 |
| KV313_HUMAN | 0 | | | | | | | | |
| KV113_HUMAN | 0 | P0DP09 | IGKV1-13 | | | | | | |
| KV204_HUMAN | 0 | | | | | | | | |

Table 1 provides non-limiting exemplary genes encoding disease labeled proteins. Classification model (Random Forest) weights of each protein and their scores against Open Targets at multiple points in the EFO Ontology are provided. FIG. 2 shows a plot of the trained classification model of cancer vs. disease labeled proteins in Table 1. The lower right quadrant represents proteins that have a significant weight in the classification but with little existing knowledge associating the proteins to the specified disease. Therefore, these proteins are suitable candidates for novel biomarkers. The upper left quadrant represents proteins that have a large body of evidence linking them to the disease but weak classification weights. These proteins could represent either a common mechanism or set of mechanisms for multiple diseases which the classification weights cannot differentiate, or alternatively, could represent a weak role in the classification of the disease. The upper right and lower left quadrants help support the validity of the classification model. FIG. 3 is a bar graph of classification model weight, represents all proteins in Table 1 detected in the broad range proteome sampling, ordered in descending classification model weight. The top plot represents the top 50 proteins (by classification model weight), and the bottom plot shows all proteins in Table 1. The existing knowledge for association to disease is high if that protein is well associated to the disease through existing knowledge. Proteins with low existing knowledge for association to disease represent suitable candidates for potential biomarkers as they represent proteins with a significant classification weight but little existing knowledge.

In some embodiments, classification model weight and importance for each protein in each disease label, the definition depends on the algorithm used. The classification model weight of Table 1 is generated using Random Forest. Random Forest allows estimation of the classification model weight and importance for each protein by a number of methods, one being removing or perturbing the values. The average of the resulting changes in classification error provides an indication of weight. The classification model weights are dependent on the dataset and are relative within the dataset. For example, if a feature (protein) is removed from a data set, the weights of the other feature are recalculated when the model is retrained. For example, if the same algorithm is used on a similar but a new set of data, the weight can be different. The methods disclosed herein provide stability of important features across many independent datasets by focusing on the subset of the important features consistent across different datasets. The methods provided herein identify the subset currently not related to known knowledge as a novel biomarker.

### Trained Classification Model

Present disclosure relates to methods of identifying biomarkers (e.g., protein) that are linked to a specified biological state (e.g., a disease state) and are present in a very low or non-recorded concentration in presently known databases. The present disclosure also relates to computer implemented machine learning classification algorithms, apparatuses, systems, and computer readable media for assessing a likelihood that a patient has the specified biological state, such as cancer, relative to a patient (test) population or a control (reference) population.

### Machine Learning Algorithm Based Methods

The collection of data from patient samples presents a very complex network of information about the patient. This complex network of information can be effectively untangled by modem machine learning algorithms. Modem machine learning and artificial intelligence algorithms are well suited to managing large quantities of heterogeneous data.

The term “machine learning” refers to algorithms that give a computer the ability to learn without being explicitly programmed including algorithms that can learn from and make predictions about data. Non-limiting exemplary machine learning algorithms include decision tree learning, artificial neural networks, deep learning neural network, support vector machines, rule base machine learning, random forest, nearest neighbor, support vector classifier, partial least square, and logistic regression. Non-limiting examples of neural networks include convolutional neural networks, deep convolutional neural networks, cascaded deep convolutional neural networks, graph convolutional neural networks (GCNN), etc. In some embodiments, algorithms such as linear regression or logistic regression can be used as part of a machine learning process. However, it is understood that using linear regression or another algorithm as part of a machine learning process is distinct from performing a statistical analysis such as regression with a spreadsheet program such as Excel. The machine learning process has the ability to continually learn and adjust the classifier as new data becomes available, and does not rely on explicit or rules-based programming. Statistical modeling relies on finding relationships between variables (e.g., mathematical equations) to predict an outcome.

Machine learning has two main phases: a training phase and an application or testing phase. During training, models are learned from labelled diseases and their respective association scores. During the testing or application phase, the models are then applied to unseen data for classifying against the biological states used in training.

Generating a prediction algorithm by training a machine is a well-known technique. The most important in the training of the machine is the quality of the database used for the training. Typically, the machine combines one or more linear models, support vector machines, decision trees and/or a neural network.

Machine learning can be generalized as the ability of a learning machine to perform accurately on new, unseen examples/tasks after having experienced a learning data set. Machine learning can include the following concepts and methods. Supervised learning concepts can include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher's linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ, SPRINT; Bayesian networks, such as Naive Bayes; and Hidden Markov models. Unsupervised learning concepts can include; Expectation-maximization algorithm; Vector Quantization; Generative topographic map; Information bottleneck method; Artificial neural network, such as Self-organizing map; Association rule learning, such as, Apriori algorithm, Eclat algorithm, and FP-growth algorithm; Hierarchical clustering, such as Single-linkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts can include; Generative models; Low-density separation; Graph-based methods; and Co-training. Reinforcement learning concepts can include; Temporal difference learning; Q-learning; Learning Automata; and SARSA. Deep learning concepts can include; Deep belief networks; Deep Boltzmann machines; Deep Convolutional neural networks; Deep Recurrent neural networks; and Hierarchical temporal memory.

In some embodiments, the present systems and methods relate to generating a trained classification model by assigning classification model weight and existing knowledge association score. In some embodiments, the variables used to train the machine comprise the expression level of biomolecules (e.g., protein) from a sample. In the present disclosure, the machine learning algorithms are “trained” by building a trained classification model from inputs. The inputs can be retrospective data with a known diagnosis of cancer (including matched controls) and data from measured biomarkers and clinical factors of those patients. In some embodiments, the inputs can be complex biomolecule sampling data from a test subject who has or is currently undergoing any form of treatment (e.g., cancer treatment). In some embodiments, the subject is diagnosed with or suspected of having or developing a disease or disorder. In some embodiments, the disease is cancer. In some embodiments, the subject is in cancer remission.

In some embodiments, the surface-activity relationships (i.e., intermolecular interaction modeling) of the biomarkers identified and the corona which captured them are stored in a relational database and applied to a graph convolutional neural network (GCNN) to rationally design coronas with features configured to target specific biomarkers which may be relevant to a disease state.

In some embodiments, broad range sampling of complex biomolecules without any prior depletion generates a large training data set and test data set of disease labeled biomolecules (e.g., protein) across many patient samples. The trained machine learning algorithm can assign a classification model weight to each biomolecule (e.g, protein) in the test data set and all known biomolecules for a specified biological state (e.g., cancer). The classification strength of the biomolecules can be determined by assigning an existing knowledge association score (FIG. 2). The classification strength of a biomolecule (e.g., protein as a biomarker of a cancer) can classify a biomarker into a category indicative of a likelihood that the biomarker plays a role in the specified biological state (e.g., a disease state). The categories can be divided into four subgroups: (1) having a significant classification model weight but with little existing knowledge association for a specified biological state (e.g., a disease state) (the lower right quadrant of FIG. 2); (2) having a significant classification model weight with well-known existing knowledge association for a specified biological state (e.g., a disease state) (the upper right quadrant of FIG. 2); (3) having a weak classification model weight with little existing knowledge association for a specified biological state (e.g., a disease state) (the lower left quadrant of FIG. 2); and (4) having a weak classification model weight with well-known existing knowledge association for a specified biological state (e.g., a disease state) (the upper left quadrant of FIG. 2). The biomolecules assigned to a category of having a significant classification model weight but with little existing knowledge association for a specified biological state (e.g., a disease state) (the lower right quadrant of FIG. 2) may be novel biomarkers associated with the specified biological state.

It is understood that the basis of use of the biomarkers identified by presently described methods for e.g., early detection of cancer and/or monitoring of a treatment response to a therapy is based on, at least in part, (1) an identification of a specified biological state (e.g., a type of cancer), (2) validated biomarkers that are associated with the specified biological state, (3) clinical parameter data, and in some cases, (4) publically available data including risk factors for having the cancer. Validation of the biomarkers identified in the present methods can be provided by analyzing retrospective e.g., cancer samples along with age matched normal (e.g., non-cancer) samples and/or other controls.

### Specificity and Sensitivity

Sensitivity and specificity are statistical measures of the performance of a binary classification test, also known in statistics as classification function. Sensitivity measures the proportion of positives that are correctly identified as such (e.g. the percentage of subject with a disease or disorder who are correctly identified as having the disease or disorder). Specificity measures the proportion of negatives that are correctly identified as such (e.g. the percentage of subject without the disease or disorder who are correctly identified as not having the disease or disorder). Sensitivity quantifies the avoiding of false negatives, and specificity does the same for false positives.

Sensitivity and specificity are prevalence-independent test characteristics, as their values are intrinsic to the test and do not depend on the disease prevalence in the population of interest. Positive and negative predictive values, but not sensitivity or specificity, are values influenced by the prevalence of disease in the population that is being tested. Bayesian clinical diagnostic model can demonstrate the positive and negative predictive values as a function of the prevalence, the sensitivity and specificity. Bayesian inference is a method of statistical inference in which Bayes' rule is used to update the probability that a hypothesis is correct as evidence is added. In clinical medicine, Bayesian methods are used to establish the probability that a patient has a particular condition given the results of the test used and the prevalence of the condition in the population tested. The probability that the subject has the condition is largely dependent on the frequency of the condition in the population tested (prevalence). This applies even if the test has a high probability of being correct (sensitivity) and a high probability of identifying the patients that do not have the condition (specificity).

The tradeoff between specificity and sensitivity can be represented graphically using a receiver operating characteristic (ROC) curve. ROC curve is a graphical plot that illustrates the performance of a binary classifier system as its discrimination threshold is varied. ROC curve analysis is often applied to measure the diagnostic accuracy of a biomarker. The analysis results in two gains: diagnostic accuracy of the biomarker and the optimal cut-point value. The ROC curve is a mapping of the sensitivity versus for all possible values of the cut-point between cases and controls.

To measure the diagnostic ability of a biomarker, it is common to use summary measures such as the area under the ROC curve (AUC) and/or the partial area under the ROC curve (pAUC). A biomarker with AUC=1 discriminates individuals perfectly as diseased or healthy. Meanwhile, an AUC=0.5 means that there is no apparent distributional difference between the biomarker values of the two groups. ROC analysis provides two main outcomes: the diagnostic accuracy of the test and the optimal cut-point value for the test. Cut-points dichotomize the test values, so this provides the diagnosis (diseased or not). The identification of the cut-point value requires a simultaneous assessment of sensitivity and specificity. A cut-point is referred to as optimal when the point classifies most of the individuals correctly. AUC, sensitivity, and specificity values are useful for the evaluation of a marker; however, they do not specify “optimal” cut-points directly. The ROC curve is created by plotting the true positive rate (sensitivity) against the false positive rate (1—specificity) at various threshold settings. The true-positive rate is also known as sensitivity, recall or probability of detection in machine learning. The false-positive rate is also known as the fall-out or probability of false alarm and can be calculated as (1—specificity). The ROC curve is thus the sensitivity as a function of fall-out. In general, if the probability distributions for both detection and false alarm are known, the ROC curve can be generated by plotting the cumulative distribution function (area under the probability distribution from-∞ to the discrimination threshold) of the detection probability in the y-axis versus the cumulative distribution function of the false-alarm probability on the x-axis. ROC analysis provides tools to select possibly optimal models and to discard suboptimal ones independently from (and prior to specifying) the cost context or the class distribution. ROC analysis is related in a direct and natural way to cost/benefit analysis of diagnostic decision making.

Area under the ROC curve (AUC) means a statistic to measure classifier performance, commonly used in machine learning applications that encapsulate both sensitivity and specificity of the classifier performance. In a ROC curve, the true positive rate (sensitivity) is plotted in function of the false positive rate (1—specificity) for different cut-off points of a parameter. Each point on the ROC curve represents a sensitivity/specificity pair corresponding to a particular decision threshold. The area under the ROC curve (AUC) is a measure of how well a parameter can distinguish between two diagnostic groups (diseased/normal).

Positive predictive value (PPV) and negative predictive value (NPV) are the proportions of positive and negative results in statistics and diagnostic tests that are true positive and true negative results, respectively. The PPV and NPV describe the performance of a diagnostic test or other statistical measure. A high result can be interpreted as indicating the accuracy of such a statistic. Although sometimes used synonymously, a negative predictive value generally refers to what is established by control groups, while a negative post-test probability rather refers to a probability for an individual. Still, if the individual's pre-test probability of the target condition is the same as the prevalence in the control group used to establish the negative predictive value, then the two are numerically equal.

The methods provided herein can identify one or more biomarkers with a significant classification model weight with little existing knowledge association for a specified biological state (e.g., a disease state) with a specificity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9%. The methods provided herein can identify one or more biomarkers with a significant classification model weight with little existing knowledge association for a specified biological state (e.g., a disease state) with a specificity about 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9%.

The methods provided herein can identify one or more biomarkers with a significant classification model weight with little existing knowledge association for a specified biological state (e.g., a disease state) with a sensitivity of at least 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9%. The methods provided herein can identify one or more biomarkers with a significant classification model weight with little existing knowledge association for a specified biological state (e.g., a disease state) with a sensitivity about 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9%.

In some applications, the one or more biomarkers with a significant classification model weight with little existing knowledge association for a specified biological state (e.g., a disease state) can indicates an increased likelihood of one or more of: a poor clinical outcome, good clinical outcome, high risk of disease, low risk of disease, complete response, partial response, stable disease, non-response, and recommended treatments for disease management.

The methods provided herein can identify one or more biomarkers with a significant classification model weight with little existing knowledge association for a specified biological state (e.g., a disease state) with a specificity greater than 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% ROC. The methods provided herein can identify one or more biomarkers with a significant classification model weight with little existing knowledge association for a specified biological state (e.g., a disease state) with a sensitivity greater than 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, or 99.9% ROC.

### Computer Systems

The current disclosure provides computer systems for implementing any of the methods described herein. A computer system can be used to implement one or more steps including, sample collection, sample processing, detecting, quantifying one or more biomolecules, generating a profile data, comparing said data to a reference, generating a subject-specific biomolecule profile, comparing the subject-specific profile to a reference profile, receiving medical history, receiving medical records, receiving and storing data obtained by one or more methods described herein, analyzing said data, generating a report, and reporting results to a receiver.

Computer systems described herein can comprise computer-executable code for performing any of the algorithms described herein. Computer systems described herein can comprise computer-executable code for performing any of the algorithms and using the database as herein.

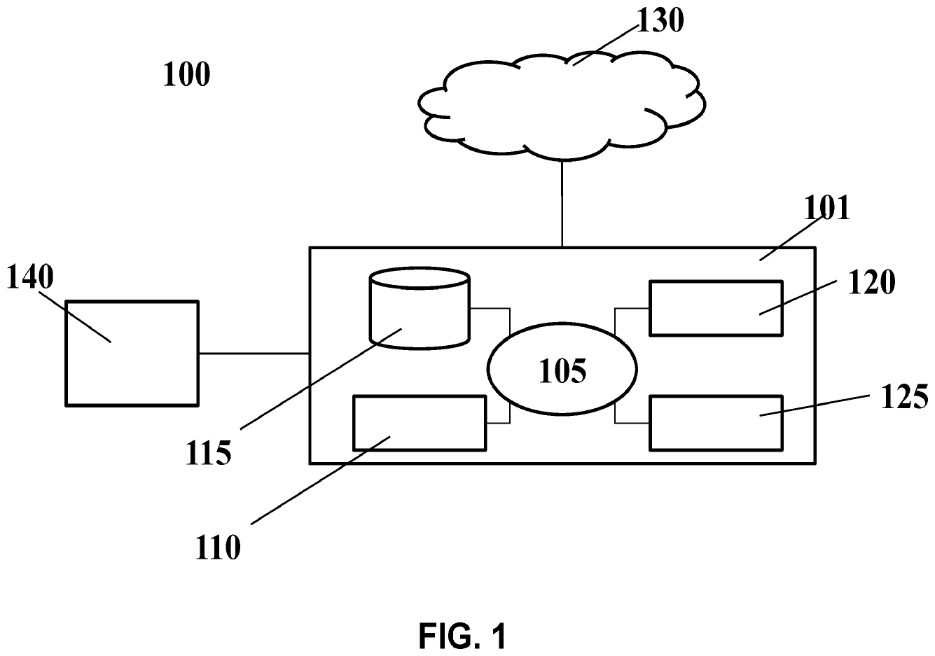

FIG. 1 depicts an exemplary computer system 100 adapted to implement a method described herein. The system 100 includes a central computer server 101 that is programmed to implement exemplary methods described herein. The server 101 includes a central processing unit (CPU, also “processor”) 105 which can be a single core processor, a multi core processor, or plurality of processors for parallel processing. The server 101 also includes memory 110 (e.g. random access memory, read-only memory, flash memory); electronic storage unit 115 (e.g. hard disk); communications interface 120 (e.g. network adaptor) for communicating with one or more other systems; and peripheral devices 125 which can include cache, other memory, data storage, and/or electronic display adaptors. The memory 110, storage unit 115, interface 120, and peripheral devices 125 are in communication with the processor 105 through a communications bus (solid lines), such as a motherboard. The storage unit 115 can be a data storage unit for storing data. The server 101 is operatively coupled to a computer network (“network”) 130 with the aid of the communications interface 120. The network 130 can be the Internet, an intranet and/or an extranet, an intranet and/or extranet that is in communication with the Internet, a telecommunication or data network. The network 130 in some cases, with the aid of the server 101, can implement a peer-to-peer network, which can enable devices coupled to the server 101 to behave as a client or a server.

The storage unit 115 can store files, such as subject reports, and/or communications with the caregiver, sequencing data, data about individuals, or any aspect of data associated with the present disclosure.

The server can communicate with one or more remote computer systems through the network 130. The one or more remote computer systems can be, for example, personal computers, laptops, tablets, telephones, Smart phones, or personal digital assistants.

In some applications the computer system 100 includes a single server 101. In other situations, the system includes multiple servers in communication with one another through an intranet, extranet and/or the internet.

The server 101 can be adapted to store measurement data or a database as provided herein, patient information from the subject, such as, for example, polymorphisms, mutations, medical history, family history, demographic data and/or other clinical or personal information of potential relevance to a particular application. Such information can be stored on the storage unit 115 or the server 101 and such data can be transmitted through a network.

Methods as described herein can be implemented by way of machine (or computer processor) executable code (or software) stored on an electronic storage location of the server 101, such as, for example, on the memory 110, or electronic storage unit 115. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110. Alternatively, the code can be executed on a second computer system 140.

Aspects of the systems and methods provided herein, such as the server 101, can be embodied in programming. Various aspects of the technology can be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which can provide non-transitory storage at any time for the software programming. All or portions of the software can at times be communicated through the Internet or various other telecommunication networks. Such communications, for example, can enable loading of the software from one computer or processor into another, for example, from a management server or host computer into the computer platform of an application server. Thus, another type of media that can bear the software elements includes optical, electrical, and electromagnetic waves, such as used across physical interfaces between local devices, through wired and optical landline networks and over various air-links. The physical elements that carry such waves, such as wired or wireless likes, optical links, or the like, also can be considered as media bearing the software. As used herein, unless restricted to non-transitory, tangible “storage” media, terms such as computer or machine “readable medium” can refer to any medium that participates in providing instructions to a processor for execution

Computer systems described herein can comprise computer-executable code or instruction for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

### Databases

Computer systems disclosed herein can comprise a memory unit. The memory unit can be configured to receive data comprising extracting data from a pubic database, detecting, quantifying and profiling one or more biomolecules.

There are several public searchable metabolome database and proteome database known in the art. The present methods of the disclosure can be used with such public databases as well as proprietary databases. Examples of public databases include but are not limited to Open Targets (opentargets.org), Gene Ontology Consortium (geneontology.org), Plasma Proteome Database (plasmaproteomedatabase.org), METLIN (metlin.scripps.edu), Human Metabolome Database (hmdb.ca), Kyoto Encyclopedia of Genes and Genomes (genome.jp/kegg/), Biological Magnetic Resonance Bank (bmrb.wisc.edu/metabolomics/), Proteomics Identifications (PRIDE) (ebi.ac.uk/pride), ProteomicsDB (proteomicsdb.org), or Biological Magnetic Resonance Bank (bmrb.wisc.edu/metabolomics/).

The Open Targets database calculates the disease association score of each protein based on evidence from various databases to score the available evidence from a scale of 0 (lowest) to 1.0 (highest) level of disease association. Non-limiting exemplary Open Target database include GWAS Catalog (D. Welter et al., The NHGRI GWAS Catalog, a curated resource of SNP-trait associations. Nucleic acids research 42, D1001-D1006 (2013)); UniProt (U. Consortium, UniProt: a hub for protein information. Nucleic acids research, gku989 (2014)): Gene2Phenotype (C. F. Wright et al., Genetic diagnosis of developmental disorders in the DDD study: a scalable analysis of genome-wide research data. The Lancet 385, 1305-1314 (2015)); Cancer Gene Census (S. A. Forbes et al., COSMIC: exploring the world's knowledge of somatic mutations in human cancer. Nucleic acids research 43, D805-D811 (2014)); IntOGen (C. Rubio-Perez et al., In silico prescription of anticancer drugs to cohorts of 28 tumor types reveals targeting opportunities. Cancer cell 27, 382-396 (2015)); Europe PMC (E. P. Consortium, Europe PMC: a full-text literature database for the life sciences and platform for innovation. Nucleic acids research, gku1061 (2014)); and Reactome (D. Croft et al., The Reactome pathway knowledgebase. Nucleic acids research 42, D472-D477 (2013)).

The Human Metabolome Database (HMDB) is a freely available electronic database containing detailed information about small molecule metabolites found in the human body. It is intended to be used for applications in metabolomics, clinical chemistry, biomarker discovery and general education. The database is designed to contain or link three kinds of data: 1) chemical data, 2) clinical data, and 3) molecular biology/biochemistry data. The database (version 3.5) contains 40,446 metabolite entries including both water-soluble and lipid soluble metabolites as well as metabolites that would be regarded as either abundant (>1 pM) or relatively rare (<1 nM). Additionally, 5,235 protein (and DNA) sequences are linked to these metabolite entries. See Wishart, D. S., Tzur, D., Knox, C., et al., HMDB: the Human Metabolome Database, Nucleic Acids Res. 2007 January; 35(Database issue):D521-6; Wishart, D. S., Knox, C., Guo, A. C., et al., HMDB: a knowledgebase for the human metabolome, Nucleic Acids Res. 2009 37(Database issue):D603-610; Wishart., D. S., Jewison, T., Guo, A. C., Wilson, M., Knox, C., et al., HMDB 3.0—The Human Metabolome Database in 2013, Nucleic Acids Res. 2013. Jan. 1; 41(D1):D801-7. The database can be located on central server containing the computer-executable code that allows access to a user. The user can connect to the central server through a physical connection or cloud-based connection depending on the application. In some applications a portion of the database and necessary executable code will be supplied to as user on appropriate storage media.

Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Computer Generated Report

The computer system can comprise computer executable instruction for providing a report communicating the detecting, measuring, or determining a sampling of complex biomolecule from a subject. Measuring, or determining a sampling of complex biomolecule can include the use of a database as provided herein.

The computer system for a complex biomolecule sampling as provided herein can comprise a first memory unit for receiving a plurality of biomolecule sampling data, wherein the plurality of biomolecule sampling data comprises a first biomolecule sampling data from a first complex biological sample and a second biomolecule sampling data from a second complex biological sample, wherein the first complex biological sample is from one or more subjects with a specified biological state and the second complex biological sample is from one or more subjects without the specified biological state; a second memory unit for querying a known biomolecule data aggregator, wherein the known biomolecule data aggregator comprises all known biomolecules associated with the specified biological state; a first computer executable instruction for building a trained classification model by extracting a first feature of the first biomolecule sampling data and a second feature of the second biomolecule sampling data, wherein the trained classification model of the first feature and the second feature identifies one or more biomarkers linked to the specified biological state; and a second computer executable instruction for processing the trained classification model against the known biomolecule data aggregator and assigning a classification weight to all biomolecules, wherein said processing and assigning identifies one or more biomarkers linked to the specified biological state, wherein the one or more biomarkers confirms the specified biological state with a high sensitivity and a high specificity, wherein the one or more biomarker is present in a low or non-recorded concentration in the first complex biological sampling data.

The computer system herein can further comprising a third computer executable instruction for generating a report of the presence or absence of the specified biological state in a subject. The report can comprise a recommended treatment for a disease management. The computer system can further comprise a user interface configured to communicate or display said report to a user.

### Systems for Generating Complex Biomolecule Sampling Data

Techniques and systems to produce broad range sampling of complex biomolecules (e.g., plasma proteome) without a prior depletion and independent of e.g., plasma protein concentration and to generate a large training and test data set of disease labeled biomolecule (e.g., protein) levels across many patient sample are described in International Patent Application PCT/US2017/067013, which is herein incorporated by reference in its entirety.

### Nanoparticles

In some embodiments, the system can comprise multi-particle enabled complex biomolecule sampling. In some embodiments, the particles are nanoparticles. In some embodiments, the particles are liposomes. The liposomes can comprise any lipid capable of forming a particle. In one embodiment, the liposome comprises one or more cationic lipids or anionic lipids, and one or more stabilizing lipids. Suitable liposomes are known in the art and include, but are not limited to, for example, DOPG (1,2-dioleoyl-sn-glycero-3-phospho-(1′-rac-glycerol), DOTAP(1,2-Dioleiyl-3 trimethylammonium-propane)-DOPE (dioleoylphosphatidylethanolamine), CHOL (DOPC-Cholesterol), and combinations thereof.

The lipid-based surface of a liposome can contact a subset of biomolecules (e.g., proteins) of a complex biological sample (e.g., plasma, or any sample having a complex mix of biomolecules such as proteins and nucleic acid and at least one of a polysaccharide and lipid) at a lipid-biomolecule (e.g. protein) interface, thereby binding the subset of proteins to produce a pattern of biomolecule (e.g. protein) binding.

In one embodiment, the liposome comprises a cationic lipid. As used herein, the term “cationic lipid” refers to a lipid that is cationic or becomes cationic (protonated) as the pH is lowered below the pK of the ionizable group of the lipid, but is progressively more neutral at higher pH values. At pH values below the pK, the lipid is then able to associate with negatively charged nucleic acids. In certain embodiments, the cationic lipid comprises a zwitterionic lipid that assumes a positive charge on pH decrease. In certain embodiments, the liposomes comprise cationic lipid. In some embodiments, cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethy-lammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), N-(1,2-dimyristyloxyprop-3-yl)-N,N-dimethyl-N-hydroxyethyl ammonium bromide (DMRIE), 1,2-dioleoyl-sn-3-phosphoethanolamine (DOPE), N-(1-(2,3-dioleyloxy)propyl)-N-(2-(sperminecarboxamido)ethyl)-N,N-dimethy-lammonium trifluoroacetate (DOSPA), dioctadecylamidoglycyl carboxyspermine (DOGS), and 1,2-ditetradecanoyl-sn-glycero-3-phosphocholine (DMPC). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA). In some embodiment, the lipid is an amino lipid.

In certain embodiments, the liposome comprises one or more additional lipids which stabilize the formation of particles during their formation. Suitable stabilizing lipids include neutral lipids and anionic lipids. The term “neutral lipid” refers to any one of a number of lipid species that exist in either an uncharged or neutral zwitterionic form at physiological pH. Representative neutral lipids include diacylphosphatidylcholines, diacylphosphatidylethanolamines, ceramides, sphingomyelins, dihydro sphingomyelins, cephalins, and cerebrosides. Exemplary neutral lipids include, for example, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoyl-phosphatidyethanol amine (SOPE), and 1,2-dielaidoyl-sn-glycero-3-phophoethanolamine (transDOPE). In one embodiment, the neutral lipid is 1,2-distearoyl-sn-glycero-3-phosphocholine (DSPC).

The term “anionic lipid” refers to any lipid that is negatively charged at physiological pH. These lipids include phosphatidylglycerol, cardiolipin diacylphosphatidylserine, diacylphosphatidic acid, N-dodecanoylphosphatidylethanolamines, N-succinylphosphatidylethanolamines, N-glutarylphosphatidylethanolamines, lysylphosphatidyiglycerols, palmitoyloleyolphosphatidylglycerol (POPG), and other anionic modifying groups joined to neutral lipids. In certain embodiments, the liposome comprises glycolipids (e.g., monosialoganglioside GM.sub.1). In certain embodiments, the liposome comprises a sterol, such as cholesterol. In certain embodiments, the liposome comprises an additional, stabilizing-lipid which is a polyethylene glycol-lipid. Suitable polyethylene glycol-lipids include PEG-modified phosphatidylethanolamine, PEG-modified phosphatidic acid, PEG-modified ceramides (e.g., PEG-CerC14 or PEG-CerC20), PEG-modified dialkylamines, PEG-modified diacylglycerols, PEG-modified dialkylglycerols. Representative polyethylene glycol-lipids include PEG-c-DOMG, PEG-c-DMA, and PEG-s-DMG. In one embodiment, the polyethylene glycol-lipid is N-[(methoxy poly(ethylene glycol).sub.2000)carbamyl]-1,2-dimyristyloxlpropyl-3-amine (PEG-c-DMA). In one embodiment, the polyethylene glycol-lipid is PEG-c-DOMG).

Suitable liposomes can be solid lipid nanoparticles (SLN) which can be made of solid lipid, emulsifier and/or water/solvent. SLN can include, but are not limited to, a combination of the following ingredients: triglycerides (tri-stearin), partial glycerides (Imwitor), fatty acids (stearic acid, palmitic acid), and steroids (cholesterol) and waxes (cetyl palmitate). Various emulsifiers and their combination (Pluronic F 68, F 127) have been used to stabilize the lipid dispersion. Suitable ingredients for the use in preparing SNL sensor elements include, but are not limited to, e.g., phospholipids, glycerol, poloxamer 188, soy phosphatidyl choline, compritol, cetyl palmitate, PEG 2000, PEG 4500, Tween 85, ethyl oleate, Na alginate, ethanol/butanol, tristearin glyceride, PEG 400, isopropyl myristate, Pluronic F68, Tween 80, trimyristin, tristearin, trilaurin, stearic acid, glyceryl caprate as Capmul®MCM C10, theobroma oil, triglyceride coconut oil, 1-octadecanol, glycerol behenate as Compritol® 888 ATO, glycerol palmitostearate as Precirol® ATO 5, and cetyl palmitate wax and the like.

Suitable nanoparticles are known in the art and include, but are not limited to, for example, natural or synthetic polymers, copolymers, terpolymers (with the cores being composed of metals or inorganic oxides, including magnetic cores). Suitable polymeric nanoparticles include, but are not limited to, e.g., polystyrene; poly(lysine), chitosan, dextran, poly(acrylamide) and its derivatives such as N-isopropylacrylamide, N-tertbutylacrylamide, N,N-dimethylacrylamide, polyethylene glycol, poly(vinyl alcohol), gelatin, starch, degradable (bio)polymers, silica and the like.

In various embodiments, the core of the nanoparticles can include an organic particle, an inorganic particle, or a particle including both organic and inorganic materials. For example, the particles can have a core structure that is or includes a metal particle, a quantum dot particle, a metal oxide particle, or a core-shell particle. For example, the core structure can be or include a polymeric particle or a lipid-based particle, and the linkers can include a lipid, a surfactant, a polymer, a hydrocarbon chain, or an amphiphilic polymer. For example, the linkers can include polyethylene glycol or polyalkylene glycol, e.g., the first ends of the linkers can include a lipid bound to polyethelene glycol (PEG) and the second ends can include functional groups bound to the PEG. In these methods, the first or second functional groups can include an amine group, a maleimide group, a hydroxyl group, a carboxyl group, a pyridylthiol group, or an azide group.

In certain embodiments, the nanoparticles can comprise polymers that include, for example, a sodium polystyrene sulfonate (PSS), polyethylene oxide (PEO), polyoxyethylene glycol, polyethylene glycol (PEG), polyethylene imine (PEI), polylactic acid, polycaprolactone, polyglycolic acid, poly(lactide-co-glycolide polymer (PLGA), cellulose ether polymer, polyvinylpyrrolidone, vinyl acetate, polyvinylpyrrolidone-vinyl acetate copolymer, polyvinyl alcohol (PVA), acrylate, polyacrylic acid (PAA), vinyl acetate, crotonic acid copolymers, polyacrylamide, polyethylene phosphonate, polybutene phosphonate, polystyrene, polyvinylphosphonate, polyalkylene, carboxy vinyl polymer, sodium alginate, carrageenan, xanthan gum, gum acacia, Arabic gum, guar gum, pullulan, agar, chitin, chitosan, pectin, karaya gum, locust bean gum, maltodextrin, amylose, corn starch, potato starch, rice starch, tapioca starch, pea starch, sweet potato starch, barley starch, wheat starch, hydroxypropylated high amylose starch, dextrin, levan, elsinan, gluten, collagen, whey protein isolate, casein, milk protein, soy protein, keratin, or a gelatin, or a copolymer, derivative, or mixture thereof.

In other embodiments, the polymer can be or include a polyethylene, polycarbonate, polyanhydride, polyhydroxyacid, polypropylfumerate, polycaprolactone, polyamide, polyacetal, polyether, polyester, poly(orthoester), polycyanoacrylate, polyvinyl alcohol, polyurethane, polyphosphazene, polyacrylate, polymethacrylate, polycyanoacrylate, polyurea, polystyrene, or a polyamine, or a copolymer, derivative, or mixture thereof.

In some embodiments, the present disclosure provides nanoparticles comprising biodegradable polymers. The non-limiting exemplary biodegradable polymers can be poly-β-amino-esters (PBAEs), poly(amido amines), polyesters including poly lactic-co-glycolic acid (PLGA), polyanhydrides, bioreducible polymers, and other biodegradable polymers. In some embodiments, the biodegradable polymer comprises 2-(3-aminopropylamino)ethanol end-modified poly(1,4-butanediol diacrylate-co-4-amino-1-butanol), (1-(3-aminopropyl)-4-methylpiperazine end-modified poly(1,4-butanediol diacrylate-co-4-amino-1-butanol), 2-(3-aminopropylamino)ethanol end-modified poly(1,4-butanediol diacrylate-co-5-amino-1-pentanol), (1-(3-aminopropyl)-4-methylpiperazine end-modified poly(1,4-butanediol diacrylate-co-5-amino-1-pentanol), 2-(3-aminopropylamino)ethanol end-modified poly(1,5 pentanediol diacrylate-co-3-amino-1-propanol), and (1-(3-aminopropyl)-4-methylpiperazine-end-modified poly(1,5 pentanediol diacrylate-co-3-amino-1-propanol).

The plurality of nanoparticles can be in any suitable combination of two or more nanoparticles in which each nanoparticles provides a unique biomolecule corona signature. For example, the plurality of nanoparticles can include one or more liposome and one or more nanoparticle described herein. In one embodiment, the plurality of nanoparticles can be a plurality of liposomes with varying lipid content and/or varying charges (cationic/anionic/neutral). In another embodiment, the plurality of nanoparticles can contain one or more nanoparticle made of the same material but of varying sizes and physiochemical properties.

The physicochemical properties include, but are not limited to the composition, size, surface charge, hydrophobicity, hydrophilicity, surface functionality (surface functional groups), surface topography, surface curvature and shape. The term composition encompasses the use of different types of materials and differences in the chemical and/or physical properties of materials, for example, conductivity of the material chosen between the sensor elements.

Surface curvature is generally determined by the nanoparticle size. Thus, at a nanometer scale, as the size of the surface curvature affects the binding selectivity of the surface. For example, at certain curvature, the surface of the particle may have a binding affinity for a specific type of biomolecule where a different curvature will have a different binding affinity and/or a binding affinity for a different biomolecule. The curvature can be adjusted to create a plurality of sensor elements with altered affinity for different biomolecules. A sensor array can be created including a plurality of sensor elements having different curvatures (e.g. different sizes) which results in a plurality of sensor elements each with a different biomolecule corona signature.

In another embodiment, the plurality of nanoparticles can contain one or more nanoparticle made of differing materials (e.g. silica and polystyrene) with similar or varying sizes and/or physiochemical properties (e.g. modifications, for example, —NH2, —COOH functionalization). These combinations are purely provided as examples and are non-limiting to the scope of the present disclosure.

Surface morphology may also be modified by methods such as patterning the surface to provide different affinities, engineering surface curvatures on multiple length scales and the like. Patterning the surface is provided by, for example, forming the sensor elements by block polymerization in which the at least two blocks have different chemistries, forming the nanoparticles using mixtures of at least two different polymers and phase separating the polymers during polymerization, and/or cross-linking the separate polymers following phase separation. Engineered surface curvature on multiple length scales is provided, for example, by employing Pickering emulsions (Sacanna et al. 2007) stabilized by finely divided particles for the synthesis of nanoparticles. In some embodiments, finely divided particles are selected from, for example, silicates, aluminates, titanates, metal oxides such as aluminum, silicon, titanium, nickel, cobalt, iron, manganese, chromium, or vanadium oxides, carbo blacks, and nitrides or carbides, e.g., boron nitride, boron carbide, silicon nitride, or silicon carbide, among others. In some embodiments, finely divided particles can comprise an inorganic material. In some embodiments, finely divided particles can comprise an organic material. In some embodiments, finely divided particles can comprise biomolecules such as protein-based particles and oligonucleotide-based particles (RNA and/or DNA). In some embodiments, finely divided particles are selected from, for example, superparamagnetic materials such as magnetite, maghemite, etc. In some embodiments, finely divided particles are selected from a polymer, a metal oxide, a plasmonic material, a biomolecule, a superparamagnetic material, magnetite, maghemite, a micelle, a liposome, iron oxide, graphene, silica, polystyrene, silver, gold, a quantum dot, palladium, platinum, titanium, and any combination thereof.

Nanoparticle elements may be functionalized to have different physicochemical properties. Suitable methods of functionalizing the sensor elements are known in the art and depend on composition of the sensor element (e.g. gold, iron oxide, silica, silver, etc.), and include, but not limited to, for example aminopropyl functionalized, amine functionalized, boronic acid functionalized, carboxylic acid functionalized, amine functionalized, boronic acid functionalized, carboxylic acid functionalized, methyl functionalized, N-succinimidyl ester functionalized, PEG functionalized, streptavidin functionalized, methyl ether functionalized, triethoxylpropylaminosilane functionalized, thiol functionalized, PCP functionalized, citrate functionalized, lipoic acid functionalized, BPEI functionalized, carboxyl functionalized, hydroxyl functionalized, and the like. In one embodiment, the nanoparticles may be functionalized with an amine group (—NH2 or a carboxyl group (COOH). In some embodiments, the nanoscale sensor elements are functionalized with a polar functional group. Non-limiting examples of the polar functional group comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like. In some embodiments, the polar functional group is an ionic functional group. Non-limiting examples of the ionic function group comprise an ammonium group, an imidazolium group, a sulfonium group, a pyridinium group, a pyrrolidinium group, a phosphonium group. In some embodiments, the sensor elements are functionalized with a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

In other embodiments, the physicochemical properties of the nanoparticle may be modified by modification of the surface charge. For example, the surface can be modified to provide a net neutral charge, a net positive surface charge, a net negative surface charge, or a zwitterionic charge. The charge of the surface can be controlled either during synthesis of the element or by post-synthesis modification of the charge through surface functionalization. For polymeric nanoparticles, differences in charge can be obtained during synthesis by using different synthesis procedures, different charged comonomers, and in inorganic substances by having mixed oxidation states.

### Arrays

In some embodiments, the multi-particle enabled complex biomolecule sampling can comprise an array or a chip. In some embodiments, the multi-particle enabled complex biomolecule sampling can comprise, consist essentially of, or consist of a plurality of half particles of different geometric shapes which can be made by molding technology, 3D printing or 4D printing. Suitable half particles are known in the art, and include, but are not limited to half and partial particles in any geometric shape, for example, spheres, rods, triangles, cubes and combinations thereof. Suitably, in one embodiment, the plurality of half particles has different physicochemical properties made by 3-D printing.

In some embodiments, the particles of the multi-particle enabled complex biomolecule sampling are made by 3D or 4 D printing. Suitable methods of 3D and 4D printing, including nanoscale sensor elements are known in the art. Suitable material for 3D and 4D printing include, but is not limited to, e.g., plastics and synthetic polymers (e.g., poly-ethylene glycol-diacrylate (PEG-DA), poly (e-caprolactone) (PCL), poly(propylene oxide (PPO), poly(ethylene oxide) (PEO) etc.), metals, powders, glass, ceramics, and hydrogels. Suitable shapes made by 3D or 4D printing include, but are not limited to, for example, full or partial spheres (e.g. ¾ or half spheres), rods, cubes, triangles or other geometrical or non-geometrical shapes.

3D printing techniques include, but are not limited to, microextrusion printing, inkjet bioprinting, laser-assisted bioprinting, stereolithography, omnidirectional printing, and stamp printing.

In some embodiments, the array comprises a substrate and a plurality of nanoparticles. Regardless of the identity of the plurality of nanoparticles, this disclosure can be embodied by a matrix of plurality of nanoparticles immobilized on, connected with and/or coupled to a solid substrate. The substrate can comprise, consist essentially of or consist of polydimethylsiloxane (PDMS), silica, gold or gold coated substrate, silver or silver coated substrate, platinum or platinum coated substrate, zinc or zinc coated substrate, carbon coated substrate and the like. One skilled in the art would be able to select an appropriate substrate for the plurality of nanoparticles. In some embodiments, the plurality of nanoparticles and the substrate are made of the same element, for example, gold. In some embodiments, the substrate and nanoparticles form a chip.

In some embodiments, the array comprises a single surface, plate or chip containing two or more discrete sensor elements (regions) with topological differences that allows for discrete biomolecule corona formation at each discrete element (region). The surface plate or chip can be fabricated to include the two or more discrete elements (regions) by the methods described herein. The discrete regions can be raised surfaces of differing geometric shapes, differing sizes or differing charges or other topological differences that result in discrete sensor elements with ability to form discrete biomolecule coronas.

In some embodiments, the plurality of nanoparticles are non-covalently attached to the substrate. Suitable methods of non-covalent attachment are known in the art and include, but are not limited to, for example, metal coordination, charge interaction, hydrophobic-hydrophobic interaction, chelation and the like. In other embodiments, the plurality of nanoparticles are covalently attached to the substrate. Suitable methods of covalently linking the plurality of nanoparticles and the substrates include, but are not limited to, for example, click chemistry, irradiation, and the like.

For example, the plurality of nanoparticles can be conjugated to a substrate (e.g. silica substrate) via the amidation reaction between the amino groups on silica substrate surface and carboxylic acid groups on nanoparticle surface, via the ring-opening reaction between the epoxy groups on silica substrate surface and amino groups on nanoparticle surface, via the Michael Addition reaction between the maleimide groups on silica substrate surface and thiol or amino groups on nanoparticle surface, via the urethane reaction between the isocyanate groups on silica substrate surface and hydroxyl or amino groups on nanoparticle surface, via the oxidation reaction between the thiol groups on silica substrate surface and the ones on nanoparticle surface, via the “Click” chemistry between azide groups on silica substrate surface and alkyne groups on nanoparticle surface, via the thiol exchange reaction between 2-pyridyldithiol groups on silica substrate surface and thiol groups on nanoparticle surface, via the coordination reaction between boronic acid groups on silica substrate surface and diol groups on nanoparticle surface, via the UV light-irradiated addition reaction between C═C bonds on substrate surface and C═C bonds on nanoparticle surface and the like. Suitable methods of conjugating sensor elements to gold substrate are known in the art and include, for example, conjugation via Au-thiol bonds, via the amidation reaction between the carboxylic acid groups on gold substrate surface and the amino groups on nanoparticle surface, via “Click” chemistry between the azide groups on gold substrate surface and the alkyne groups on nanoparticle surface, via urethane reaction between the NHS groups on gold substrate surface and the amino groups on nanoparticle surface, via the ring-opening reaction between the epoxy groups on gold substrate surface and amino groups on nanoparticle surface, via the coordination reaction between boronic acid groups on silica substrate surface and diol groups on nanoparticle surface, via the UV light-irradiated addition reaction between C═C bonds on gold substrate surface and C═C bonds on nanoparticle surface, via the “Ligand-Receptor” interaction between biotin on gold substrate surface and avidin on nanoparticle surface, via the “Host-Guest” interaction between a-cyclodextrin (a-CD) on gold substrate surface and adamantine (Ad) on nanoparticle surface, and the like.

The plurality of nanoparticles can be attached to the substrate randomly or in a distinct pattern. The plurality of nanoparticles can be substantially uniformly positioned. The pattern of the arranged plurality of nanoparticles can vary according to the pattern in which the plurality of nanoparticles are attached to the substrate. Each nanoparticle t is separated by a distance. The distance between the nanoparticles arranged on the substrate can vary depending on the length of the linker used to attach or other fabrication conditions. According to various embodiments, the plurality of nanoparticles on the array can be fabricated having a desired inter-element distance and pattern. Suitable distinct patterns are known in the art, including, but not limited to, parallel lines, squares, circles, triangles and the like. Further, the nanoparticles can be arranged in rows, or columns. In some embodiments, the substrate is a flat substrate, in other embodiments, the substrate is in the form of microchannels or nanochannels. The plurality of nanoparticles can be contained within microchannels or nanochannels that restrict or control the flow of the sample through the sensor array. Suitable microchannels can range from 10 μm to about 100 μm in size.

In some embodiments, a channel is formed by lithography, etching, embossing, or molding of a polymeric surface. In general, the fabrication process can involve one or more of any of these processes, and different parts of the array can be fabricated using different methods and assembled or bonded together.

Lithography involves use of light or other form of energy such as electron beam to change a material. Typically, a polymeric material or precursor (e.g. photoresist, a light-resistant material) is coated on a substrate and is selectively exposed to light or other form of energy. Depending on the photoresist, exposed regions of the photoresist either remain or are dissolved in subsequent processing steps known generally as “developing.” This process results in a pattern of the photoresist on the substrate. In some embodiments, the photoresist is used as a master in a molding process. In some embodiments, a polymeric precursor is poured on the substrate with photoresist, polymerized (i.e. cured) and peeled off.

In some embodiments, the photoresist is used as a mask for an etching process. For example, after patterning photoresist on a silicon substrate, channels can be etched into the substrate using a deep reactive ion etch (DRIE) process or other chemical etching process known in the art (e.g. plasma etch, KOH etch, HF etch, etc.). The photoresist is removed, and the substrate is bonded to another substrate using one of any bonding procedures known in the art (e.g. anodic bonding, adhesive bonding, direct bonding, eutectic bonding, etc.). Multiple lithographic and etching steps and machining steps such as drilling can be included as required.

In some embodiments, a polymeric substrate can be heated and pressed against a master mold for an embossing process. The master mold can be formed by a variety of processes, including lithography and machining. The polymeric substrate is then bonded with another substrate to form channels and/or a mixing apparatus. Machining processes can be included if necessary.

In some embodiments, a molten polymer or metal or alloy is injected into a suitable mold and allowed to cool and solidify for an injection molding process. The mold typically consists of two parts that allow the molded component to be removed. Parts thus manufactured can be bonded to result in the substrate.

In some embodiments, sacrificial etch can be used to form channels. Lithographic techniques can be used to pattern a material on a substrate. This material is covered by another material of different chemical nature. This material can undergo lithography and etch processes, or other machining process. The substrate is then exposed to a chemical agent that selectively removes the first material. Channels are formed in the second material, leaving voids where the first material was present before the etch process.

In some embodiments, microchannels are directly machined into a substrate by laser machining or CNC machining. Several layers thus machined can be bonded together to obtain the final substrate. In some embodiments, the width or height of each channel ranges from approximately 1 μm to approximately 1000 μm. In some embodiments, the width or height of each channel ranges from approximately 5 μm to approximately 500 μm. In some embodiments, the width or height of each channel ranges from approximately 10 μm to approximately 100 μm. In some embodiments, the width or height of each channel a ranges from approximately 25 μm to approximately 100 μm. In some embodiments, the width or height of each channel ranges from approximately 50 μm to approximately 100 μm. In some embodiments, the width or height of each channel ranges from approximately 75 μm to approximately 100 μm. In some embodiments, the width or height of each channel ranges from approximately 10 μm to approximately 75 μm. In some embodiments, the width or height of each channel ranges from approximately 10 μm to approximately 50 μm. In some embodiments, the width or height of each channel ranges from approximately 10 μm to approximately 25 μm.

In some embodiments, the maximum width or height of a channel is approximately 1 μm, approximately 5 μm, approximately 10 μm, approximately 20 μm, approximately 30 μm, approximately 40 μm, approximately 50 μm, approximately 60 μm, approximately 70 μm, approximately 80 μm, approximately 90 μm, approximately 100 μm, approximately 250 μm, approximately 500 μm, or approximately 1000 μm.

In some embodiments, the width of each channel ranges from approximately 5 μm to approximately 100 μm. In some embodiments, the width of a channel is approximately 5 μm, approximately 10 μm, approximately 15 μm, approximately 20 μm, approximately 25 μm, approximately 30 μm, approximately 35 μm, approximately 40 μm, approximately 45 μm, approximately 50 μm, approximately 60 μm, approximately 70 μm, approximately 80 μm, approximately 90 μm, or approximately 100 μm.

In some embodiments, the height of each channel ranges from approximately 10 μm to approximately 1000 μm. In some embodiments, the height of a channel is approximately 10 μm, approximately 100 μm, approximately 250 μm, approximately 400 μm, approximately 500 μm, approximately 600 μm, approximately 750 μm, or approximately 1000 μm. In specific embodiments, the height of the channel(s) through which the sample flows is approximately 500 μm. In some embodiments, the height of the channel(s) through which the sample flows is approximately 500 μm.

In some embodiments, the length of each channel ranges from approximately 100 μm to approximately 10 cm. In some embodiments, the length of a channel is approximately 100 μm, approximately 1.0 mm, approximately 10 mm, approximately 100 mm, approximately 500 mm, approximately 600 mm, approximately 700 mm, approximately 800 mm, approximately 900 mm, approximately 1.0 cm, approximately 1.1 cm, approximately 1.2 cm, approximately 1.3 cm, approximately 1.4 cm, approximately 1.5 cm, approximately 5 cm, or approximately 10 cm. In some embodiments, the length of the channel(s) through which the sample flows is approximately 1.0 cm. In some embodiments, the length of the channel(s) through which the sample flows is approximately 1.0 cm.

Suitable time for incubating the array include, at least a few seconds, e.g. at least 10 seconds to about 24 hours, for example at least about 10 seconds, at least about 15 seconds, at least about 20 seconds, at least about 25 seconds, at least about 30 seconds, at least about 40 seconds, at least about 50 seconds, at least about 60 seconds, at least about 90 seconds, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 15 minutes, at least about 20 minutes, at least about 25 minutes, at least about 30 minutes, at least about 45 minutes, at least about 50 minutes, at least about 60 minutes, at least about 90 minutes, at least about 2 hours, at least about 3 hours, at least about 4 hours, at least about 5 hours, at least about 6 hours, at least about 7 hours, at least about 8 hours, at least about 9 hours, at least about 10 hours, at least about 12 hours, at least about 14 hours, at least about 15 hours, at least about 16 hours, at least about 17 hours, at least about 18 hours, at least about 19 hours, at least about 20 hours, and include any time and increment in between (e.g. 10 seconds, 11, 12, 13, 14, 15, 15, 17, 18, 19, 20, 21, 22, 23, 24, 25, 26, 27, 28, 29, 30, 31, 32 33,34, 35, 36, 37, 38, 39, 40, 41, 42, 43, 44, 45, 46, 47, 48, 49, 50, 51, 52, 53, 54, 55, 56, 57, 58, 59, 60 seconds, etc.; 1 minute, 2, 3, 4, 5, 6, 7, 9, 10, 11, 12, 13, 14, 15, 15, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, etc.; 1 hour, 2 hours, 3, hours, 4, hours, 5 hours, 6 hours, 7 hours, 8 hours etc.)

Further, the temperature at which the assay is performed can be determined by one skilled in the art, and incudes temperatures between about 4° C. to about 40° C., alternatively from about 4° C. to about 20° C., alternatively from about 10° C. to about 15° C., alternatively from about 10° C. to about 40° C., for example, at about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 25° C., about 30° C., about 35° C., about 37° C., etc. Suitable, the assay may be performed at room temperature (e.g. around about 37° C., for example from about 35° C. to about 40° C.).

### Kits

Aspects of the present disclosure that are described with respect to methods can be utilized in the context of the sensor array or kits discussed in this disclosure. Similarly, aspects of the present disclosure that are described with respect to the sensor array and methods can be utilized in the context of the kits, and aspects of the present disclosure that are described with respect to kits can be utilized in the context of the methods and sensor array.

This disclosure provides kits. The kits can be suitable for use in the methods described herein. Suitable kits include a kit for determining a biomolecule fingerprint for a sample comprising a sensor array as described herein. In one aspect, the kit provides a sensor array comprising at least two sensor elements which have differing physiocochemical properties from each other. In some aspects, the kits provides a comparative panel of biomolecule fingerprints in order to use the biomolecule fingerprint to determine a disease state for the subject. In some aspects, instructions on how to determine the biomolecule fingerprint are included. In some suitable embodiments, the sensor arrays are provided as chip arrays in the kit.

In other aspects, kits for determining a disease state of a subject or diagnosing or prognosing a disease in a subject are provided. Suitable kits include a sensor array comprising at least two sensor elements which have differing physiocochemical properties from each other to determining a biomolecule fingerprint. Further, the kit may further include a comparative panel of biomolecule fingerprint of different disease states or different diseases or disorders. Instructions on determining the biomolecule fingerprint and analysis are provided.

It should be apparent to those skilled in the art that many additional modifications beside those already described are possible without departing from the inventive concepts. In interpreting this disclosure, all terms should be interpreted in the broadest possible manner consistent with the context. Variations of the term “comprising” should be interpreted as referring to elements, components, or steps in a non-exclusive manner, so the referenced elements, components, or steps may be combined with other elements, components, or steps that are not expressly referenced. Embodiments referenced as “comprising” certain elements are also contemplated as “consisting essentially of and “consisting of those elements. The term “consisting essentially of and “consisting of should be interpreted in line with the MPEP and relevant Federal Circuit's interpretation. The transitional phrase “consisting essentially of limits the scope of a claim to the specified materials or steps “and those that do not materially affect the basic and novel characteristic(s)” of the claimed invention. “Consisting of is a closed term that excludes any element, step or ingredient not specified in the claim.

### EXAMPLES

These examples are provided for illustrative purposes only and not to limit the scope of the claims provided herein.

### Example 1. Algorithm-Based Broad Dynamic Range Sampling of Proteome

Multi-nanoparticle proteomic sampling was performed with three different cross-reactive liposomes with various surface charges (i.e., cationic DOPG (1,2-dioleoyl-sn-glycero-3-phospho-(1′-rac-glycerol)), anionic (DOTAP(1,2-Dioleoyl-3-trimethylammonium-propane)-DOPE(dioleoylphosphatidylethanolamine)), and neutral (CHOL (DOPC-Cholesterol), as described in International Patent Application PCT/US2017/067013, which is herein incorporated by reference in its entirety. The protein composition at the surface of three liposomes was evaluated by liquid chromatography-mass spectrometry (LC-MS/MS) in which the abundance of 850+ known proteins was defined. The bottom plot of FIG. 3 shows all 850+ proteins detected in the broad range proteome sampling. Of these, proteins with no known plasma concentration were identified for each type of nanoparticles.

Machine learning classification algorithms (e.g., Partial Least Squares, Logistic Regression, Support Vector Classifier, Nearest Neighbor, or Random Forest) were applied to these proteins to build a trained classification model. The features of individual proteins were extracted by the trained classification model and associated classification weights were generated and stored as a set of data. Another data set was created by querying data aggregators such as Open Targets, Gene Ontology Consortium and commercial options for all known biomolecules (e.g., proteins or expressing genes) connected with cancer and their respective association score. The classification strength of the protein to its strength of associations in public and private databases to the labeled diseases was compared for the set of potential biomarkers. The lower right quadrant of FIG. 2 represents proteins that have a significant weight in the classification but with little existing knowledge associating the proteins to the specified disease. Therefore, these proteins are suitable candidates for novel biomarkers. The upper left quadrant of FIG. 2 represents proteins that have a large body of evidence linking them to the disease but weak classification weights. These proteins could represent either a common mechanism or set of mechanisms for multiple diseases which the classification weights cannot differentiate, or alternatively, could represent a weak role in the classification of the disease. The upper right and lower left quadrants help support the validity of the classification model.

The top plot of FIG. 3 represents the top 50 proteins (Table 2) with a strong association with cancer classification, ordered in descending classification model weight. The existing knowledge for association to disease was determined to be high if a particular protein was associated to cancer through existing knowledge and exceeded a threshold association score. Proteins with low existing knowledge for association to disease represent suitable candidates for potential biomarkers as they represent proteins with a significant classification weight but little existing knowledge.

TABLE 2
Proteins with a strong classification weight and
a low existing knowledge association score

| | Classifi- | Uniprot | | |
|---|---|---|---|---|
| | cation | Protein | Expressing | Association |
| Protein Name | Weight | ID | Gene | Score* |
| CBPN_HUMAN | 13.3801 | P15169 | CPN1 | 0.0865 |
| FCN3_HUMAN | 12.6620 | O75636 | FCN3 | 0.1824 |
| SAA4_HUMAN | 12.3829 | P35542 | SAA4 | 0.0537 |
| IGHG1_HUMAN | 9.7744 | P01857 | IGHG1 | 0.0884 |
| IGHG3_HUMAN | 8.6336 | P01860 | IGHG3 | 0.0486 |
| FHR5_HUMAN | 8.4398 | Q9BXR6 | CFHR5 | 0.0199 |
| CO4B_HUMAN | 8.3374 | P0C0L5 | C4B | 0.0550 |
| IGLL5_HUMAN | 8.0768 | B9A064 | IGLL5 | 0.0292 |
| APOD_HUMAN | 5.8415 | P05090 | APOD | 0.1908 |
| ZPI_HUMAN | 5.7472 | Q9UK55 | SERPINA10 | 0.1558 |
| CPN2_HUMAN | 5.7253 | P22792 | CPN2 | 0.0727 |
| FGL1_HUMAN | 5.2565 | Q08830 | FGL1 | 0.0499 |
| FETUA_HUMAN | 5.1557 | P02765 | AHSG | 0.1744 |
| ITIH2_HUMAN | 5.1402 | P19823 | ITIH2 | 0.0678 |
| H4_HUMAN | 5.0371 | P62805 | HIST1H4D | 0.1259 |
| CO4A_HUMAN | 4.9451 | P0C0L4 | C4A | 0.0566 |
| CERU_HUMAN | 4.8875 | P00450 | CP | 0.1579 |
| CD5L_HUMAN | 4.4944 | O43866 | CD5L | 0.1065 |
| CNN2_HUMAN | 4.3733 | Q99439 | CNN2 | 0.0934 |
| HORN_HUMAN | 4.3387 | Q86YZ3 | HRNR | 0.1777 |
| PHLD_HUMAN | 4.2212 | P80108 | GPLD1 | 0.0883 |
| IGKC_HUMAN | 4.0922 | P01834 | IGKC | 0.1168 |
| MASP2_HUMAN | 3.1440 | O00187 | MASP2 | 0.1929 |
| ITIH1_HUMAN | 2.8782 | P19827 | ITIH1 | 0.0101 |
| FHR1_HUMAN | 2.6784 | Q03591 | CFHR1 | 0.0577 |
| COL10_HUMAN | 2.6146 | Q9Y6Z7 | COLEC10 | 0.0160 |
| BIN2_HUMAN | 2.5023 | Q9UBW5 | BIN2 | 0.0198 |
| SAA2_HUMAN | 2.3517 | P0DJI9 | SAA2 | 0.0544 |
| ANGL6_HUMAN | 1.9573 | Q8NI99 | ANGPTL6 | 0.0568 |
| CFAB_HUMAN | 1.9362 | P00751 | CFB | 0.1602 |
| TPIS_HUMAN | 1.9250 | P60174 | TPI1 | 0.1152 |
| IGHA2_HUMAN | 1.7940 | P01877 | IGHA2 | 0.0474 |
| APOC2_HUMAN | 1.6269 | P02655 | APOC2 | 0.0595 |
| EMIL1_HUMAN | 1.5578 | Q9Y6C2 | EMILIN1 | 0.1539 |
| SBSN_HUMAN | 1.4428 | Q6UWP8 | SBSN | 0.0962 |
| PRG4_HUMAN | 1.4038 | Q92954 | PRG4 | 0.0779 |
| PPIF_HUMAN | 1.3404 | P30405 | PPIF | 0.0817 |
| FHR2_HUMAN | 1.0226 | P36980 | CFHR2 | 0.0194 |
| A1AG1_HUMAN | 1.0083 | P02763 | ORM1 | 0.1707 |
| AMY1_HUMAN | 0.9997 | P04745 | AMY1C | 0.0606 |
| NEXN_HUMAN | 0.9762 | Q0ZGT2 | NEXN | 0.0170 |
| CALL5_HUMAN | 0.9576 | Q9NZT1 | CALML5 | 0.0579 |
| THBG_HUMAN | 0.9182 | P05543 | SERPINA7 | 0.1174 |
| IGHM_HUMAN | 0.9152 | P01871 | IGHM | 0.1188 |
| EFTU_HUMAN | 0.8604 | P49411 | TUFM | 0.1784 |
| SAMP_HUMAN | 0.7996 | P02743 | APCS | 0.0481 |
| GTR3_HUMAN | 0.7462 | P11169 | SLC2A3 | 0.1304 |
| TYB4_HUMAN | 0.7062 | P62328 | TMSB4X | 0.1934 |
| CBPQ_HUMAN | 0.6979 | Q9Y646 | CPQ | 0.1418 |
| SYUA_HUMAN | 0.6895 | P37840 | SNCA | 0.0873 |

*Association score is a maximum score from Open Targets against cancer and all child disease in the EFO ontology

1. A computer-implemented method for detecting one or more biomarkers in a multi-omic data set, comprising:
(a) providing a multi-omic data set generated from one or more complex biological samples obtained from one or more individual subjects using a plurality of two or more different populations of particles, wherein each individual subject has one or more specified biological states, wherein each population of the two or more populations of particles has different physicochemical properties, and wherein a biomolecule corona of each population is different from one another;
(b) applying a model to the multi-omic data set to generate one or more classification model weights, wᵢ . . . wₙ, for one or more features, fᵢ . . . fₙ, yielding (wᵢ, fᵢ), . . . , (wₙ, fₙ) and storing (wᵢ, fᵢ), . . . , (wₙ, fₙ);
(c) querying a reference data set for the one or more features, fᵢ . . . fₙ, to generate a set of scores, sᵢ . . . sₙ, yielding (sᵢ, fᵢ), . . . , (sₙ, fₙ) and storing (sᵢ, fᵢ), . . . , (sₙ, fₙ); and
(d) combining at least (wᵢ, fᵢ), . . . , (wₙ, fₙ) and (sᵢ, fᵢ), . . . , (sₙ, fₙ) to generate (wᵢ, sᵢ), . . . , (wₙ, sₙ) and selecting a subset of (wᵢ, sᵢ), . . . , (wₙ, sₙ) to detect one or more biomarkers linked to the one or more specified biological states.

2. The method of claim 1, wherein selecting the subset in (d) comprises filtering (wᵢ, sᵢ), . . . , (wₙ, sₙ) such that w at least meets a first threshold and s at least meets a second threshold such that the one or more biomarkers comprise a subset (wₖ, sₖ) . . . (wₘ, sₘ) of (wᵢ, sᵢ), . . . , (wₙ, sₙ).

3. The method of claim 2, wherein k≥i.

4. The method of claim 2, wherein m≤n.

5. The method of claim 1, wherein the model is trained using a set of labeled multi-omic data of a plurality of complex biological samples, wherein the labeled multi-omic data set comprises the one or more features fᵢ . . . fₙ corresponding to one or more specified biological states, bᵢ . . . bₙ, wherein the one or more features are proteins.

6. The method of claim 1, further comprising obtaining the one or more complex biological samples from the one or more individuals.

7. (canceled)

8. The method of claim 1, further comprising generating an output corresponding to a specified biological state of the one or more specified biological states.

9. The method of claim 1, wherein the reference data set is a database comprising features related to specified biological states by an association score.

10. The method of claim 1, wherein said set of scores, sᵢ . . . sₙ, are association scores between the one or more features and the one or more specified biological states.

11. The method of claim 1, wherein the one or more complex biological samples are selected from the group consisting of are plasma, serum, whole blood, amniotic fluid, cerebral spinal fluid, urine, saliva, tears, and feces.

12. The method of claim 1, wherein the multi-omic data comprises one or more selected from the group consisting of: proteomic data, genomic data, lipidomic data, glycomic data, transcriptomic data, or metabolomics data.

13. The method of claim 12, wherein the multi-omic data comprises proteomic data comprising (i) protein identifiers and (ii) specified biological states for the one or more individuals.

14. (canceled)

15. The method of claim 13, wherein the multi-omic data is generated by assaying a complex biological sample of an individual of the one or more individual subjects.

16. The method of claim 13, wherein the one or more features represent different proteins.

17. The method of claim 13, wherein the one or more complex biological samples are not subjected to protein depletion.

18. The method of claim 13, wherein the one or more complex biological samples are subjected to prior protein depletion.

19. The method of claim 1, wherein the one or more specified biological states are bᵢ . . . bₙ.

20-90. (canceled)

91. A method of analyzing a broad range sampling of a plurality of biomolecules comprising:
a. assigning an existing knowledge association score to the plurality of biomolecules in a test data set;
b. generating a classification model weight for the plurality of biomolecules based on (a); and
c. classifying each biomarker into a category indicative of a likelihood of the biomarker playing a role in the specified biological state.

92. The method of claim 91, wherein the category indicative of a likelihood of the biomarker playing a role in the specified biological state is:
a. having a significant classification model weight but with little existing knowledge association for the specified biological state; or
b. having a significant classification model weight with well-known existing knowledge association for the specified biological state; or
c. having a weak classification model weight with little existing knowledge association for the specified biological state; or
d. having a weak classification model weight with well-known existing knowledge association for the specified biological state.

93. The method of claim 92, wherein biomarkers classified as (a) are further classified as novel biomarkers associated with the specified biological state.

94-96. (canceled)
